SEQUENCE LISTING (83 sequences)

SEQ ID NO: 1 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQS

SEQ ID NO: 2 - protein, 99 aa, Artificial Sequence
  Description of Artificial Sequence Synthetic polypeptide
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVL

SEQ ID NO: 3 - protein, 127 aa, Artificial Sequence
  Description of Artificial Sequence Synthetic polypeptide
ADLVQSGAVVKKPGDSVRVSCEAQGYRFPDYIIHWIRRAPGQGPEWMGWMNPMGGQVNIPWKFQGRVSMTRDTSIETAYLDLRGLKSDDTAVYYCVRDRSNGSGKRFESSNWFLDLWGRGTAVTVQS

SEQ ID NO: 4 - protein, 99 aa, Artificial Sequence
  Description of Artificial Sequence Synthetic polypeptide
QSALTQPRSVSGSPGQSVTISCTGTHNLVSWCQHQPGRAPKLLIYDFNKRPSGVPDRFSGSGSGGTASLTISGLQDEDDAEYFCWAYEAFGGGTKLTVL

SEQ ID NO: 5 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPWKFQGRVSMTRDTSIETAYMDLRGLRSDDTAVYYCVRDRSNGSGKRFESSNWFLDLWGRGTLVTVSS

SEQ ID NO: 6 - protein, 99 aa, Artificial Sequence; identical to SEQ ID NO: 4
  Description of Artificial Sequence Synthetic polypeptide

SEQ ID NO: 7 - protein, 128 aa, Artificial Sequence
  Description of Artificial Sequence Synthetic polypeptide
QVQLVQSGAEVKKPGASVKVSCEASGYRFPDYIIHWIRRAPGQGPEWMGWMNPMGGQVNIPWKFQGRVSMTRDTSIETAYMELRGLRSDDTAVYYCVRDRSNGSGKRFESSNWFLDLWGRGTLVTVSS

SEQ ID NO: 8 - protein, 99 aa, Artificial Sequence; identical to SEQ ID NO: 4
  Description of Artificial Sequence Synthetic polypeptide

SEQ ID NO: 9 - protein, 128 aa, Artificial Sequence; identical to SEQ ID NO: 5
  Description of Artificial Sequence Synthetic polypeptide

SEQ ID NO: 10 - protein, 99 aa, Artificial Sequence
  Description of Artificial Sequence Synthetic polypeptide
QSALTQPRSVSGSPGQSVTISCTGTHNLVSWCQHHPGKAPKLMIYDFNKRPSGVPDRFSGSGSGGTASLTISGLQAEDEAEYFCWAYEAFGGGTKLTVL

SEQ ID NO: 11 - protein, 128 aa, Artificial Sequence; identical to SEQ ID NO: 7
  Description of Artificial Sequence Synthetic polypeptide

SEQ ID NO: 12 - protein, 99 aa, Artificial Sequence; identical to SEQ ID NO: 10
  Description of Artificial Sequence Synthetic polypeptide

SEQ ID NO: 13 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGAGKRFESSNWFLDLWGRGTAVTIQS

SEQ ID NO: 14 - protein, 99 aa, Artificial Sequence; identical to SEQ ID NO: 2
  Description of Artificial Sequence Synthetic polypeptide

SEQ ID NO: 15 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSDGSGKRFESSNWFLDLWGRGTAVTIQS

SEQ ID NO: 16 - protein, 99 aa, Artificial Sequence; identical to SEQ ID NO: 2
  Description of Artificial Sequence Synthetic polypeptide

SEQ ID NO: 17 - protein, 127 aa, Artificial Sequence
  Description of Artificial Sequence Synthetic polypeptide
ADLVQSGAVVKKPGDSVRISCEAQGYTFPDYIIHWIRRAPGQGPEWMGWMNPMGGQVNIPWKFQGRVSMTRDTSIETAFLDLRGLKSDDTAVYYCVRDRGDGSRRHFDSSNWFLDLWGRGTAVTIQS

SEQ ID NO: 18 - protein, 99 aa, Artificial Sequence; identical to SEQ ID NO: 2
  Description of Artificial Sequence Synthetic polypeptide

SEQ ID NO: 19 - protein, 127 aa, Artificial Sequence; identical to SEQ ID NO: 1
  Description of Artificial Sequence Synthetic polypeptide

SEQ ID NO: 20 - protein, 99 aa, Artificial Sequence
  Description of Artificial Sequence Synthetic polypeptide
QSALTQPRSVSASPGQSVTISCTGTHNLVSWAQHQPGRAPKLLIYDFNKRPSGVPDRFSGSGSGGTASLTITGLQDDDDAEYFCWAYEAFGGGTKLTVL

SEQ ID NO: 21 - protein, 127 aa, Artificial Sequence; identical to SEQ ID NO: 1
  Description of Artificial Sequence Synthetic polypeptide

SEQ ID NO: 22 - protein, 99 aa, Artificial Sequence
  Description of Artificial Sequence Synthetic polypeptide
QSALTQPRSVSASPGQSVTISCTGTHNLVSWVQHQPGRAPKLLIYDFNKRPSGVPDRFSGSGSGGTASLTITGLQDDDDAEYFCWAYEAFGGGTKLTVL

SEQ ID NO: 23 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPMGGQVNIPQKFQGRVSMTRDTSIETAFLDLRGLKSDDTAVYYCVRDRSNGSGKRFESSNWFLDLWGRGTAVTIQS

SEQ ID NO: 24 - protein, 99 aa, Artificial Sequence
  Description of Artificial Sequence Synthetic polypeptide
QSALTQPRSVSASPGQSVTISCTGTHNLVSWCQHQPGRAPKLLIYDFNKRPSGVPDRFSGSGSGGTASLTITGLQDDDDAEYFCWAYENFGGGTKLTVL

SEQ ID NO: 25 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPWKFQGRVSMTRDTSIETAYMDLRGLRSDDTAVYYCVRDRSNGAGKRFESSNWFLDLWGRGTLVTVSS

SEQ ID NO: 26 - protein, 99 aa, Artificial Sequence
  Description of Artificial Sequence Synthetic polypeptide
QSALTQPRSVSGSPGQSVTISCTGTHNLVSWAQHQPGRAPKLLIYDFNKRPSGVPDRFSGSGSGGTASLTISGLQDEDDAEYFCWAYEAFGGGTKLTVL

SEQ ID NO: 27 - protein, 128 aa, Artificial Sequence; identical to SEQ ID NO: 25
  Description of Artificial Sequence Synthetic polypeptide

SEQ ID NO: 28 - protein, 99 aa, Artificial Sequence
  Description of Artificial Sequence Synthetic polypeptide
QSALTQPRSVSGSPGQSVTISCTGTHNLVSWVQHQPGRAPKLLIYDFNKRPSGVPDRFSGSGSGGTASLTISGLQDEDDAEYFCWAYEAFGGGTKLTVL

SEQ ID NO: 29 - protein, 128 aa, Artificial Sequence
  Description of Artificial Sequence Synthetic polypeptide
QVQLVQSGAEVKKPGASVKVSCEASGYRFPDYIIHWIRRAPGQGPEWMGWMNPMGGQVNIPWKFQGRVSMTRDTSIETAYMELRGLRSDDTAVYYCVRDRSNGAGKRFESSNWFLDLWGRGTLVTVSS

SEQ ID NO: 30 - protein, 99 aa, Artificial Sequence; identical to SEQ ID NO: 28
  Description of Artificial Sequence Synthetic polypeptide

SEQ ID NO: 31 - protein, 128 aa, Artificial Sequence; identical to SEQ ID NO: 25
  Description of Artificial Sequence Synthetic polypeptide

SEQ ID NO: 32 - protein, 99 aa, Artificial Sequence
  Description of Artificial Sequence Synthetic polypeptide
QSALTQPRSVSGSPGQSVTISCTGTHNLVSWAQHHPGKAPKLMIYDFNKRPSGVPDRFSGSGSGGTASLTISGLQAEDEAEYFCWAYEAFGGGTKLTVL

SEQ ID NO: 33 - protein, 128 aa, Artificial Sequence; identical to SEQ ID NO: 25
  Description of Artificial Sequence Synthetic polypeptide

SEQ ID NO: 34 - protein, 99 aa, Artificial Sequence
  Description of Artificial Sequence Synthetic polypeptide
QSALTQPRSVSGSPGQSVTISCTGTHNLVSWVQHHPGKAPKLMIYDFNKRPSGVPDRFSGSGSGGTASLTISGLQAEDEAEYFCWAYEAFGGGTKLTVL

SEQ ID NO: 35 - protein, 128 aa, Artificial Sequence; identical to SEQ ID NO: 29
  Description of Artificial Sequence Synthetic polypeptide

SEQ ID NO: 36 - protein, 99 aa, Artificial Sequence; identical to SEQ ID NO: 34
  Description of Artificial Sequence Synthetic polypeptide

SEQ ID NO: 37 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPQKFQGRVSMTRDTSIETAYMDLRGLRSDDTAVYYCVRDRSNGAGKRFESSNWFLDLWGRGTLVTVSS

SEQ ID NO: 38 - protein, 99 aa, Artificial Sequence; identical to SEQ ID NO: 28
  Description of Artificial Sequence Synthetic polypeptide

SEQ ID NO: 39 - protein, 128 aa, Artificial Sequence; identical to SEQ ID NO: 25
  Description of Artificial Sequence Synthetic polypeptide

SEQ ID NO: 40 - protein, 99 aa, Artificial Sequence
  Description of Artificial Sequence Synthetic polypeptide
QSALTQPRSVSGSPGQSVTISCTGTHNLVSWVQHQPGRAPKLLIYDFNKRPSGVPDRFSGSGSGGTASLTISGLQDEDDAEYFCWAYENFGGGTKLTVL

SEQ ID NO: 41 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPYKFQGRVSMTRDTSIETAYMDLRGLRSDDTAVYYCVRDRSNGAGKRFESSNWFLDLWGRGTLVTVSS

SEQ ID NO: 42 - protein, 99 aa, Artificial Sequence; identical to SEQ ID NO: 28
  Description of Artificial Sequence Synthetic polypeptide

SEQ ID NO: 43 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPHKFQGRVSMTRDTSIETAYMDLRGLRSDDTAVYYCVRDRSNGAGKRFESSNWFLDLWGRGTLVTVSS

SEQ ID NO: 44 - protein, 99 aa, Artificial Sequence; identical to SEQ ID NO: 28
  Description of Artificial Sequence Synthetic polypeptide

SEQ ID NO: 45 - protein, 128 aa, Artificial Sequence; identical to SEQ ID NO: 43
  Description of Artificial Sequence Synthetic polypeptide

SEQ ID NO: 46 - protein, 99 aa, Artificial Sequence; identical to SEQ ID NO: 28
  Description of Artificial Sequence Synthetic polypeptide

SEQ ID NO: 47 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPWKFQGRVSMTRDTSIETAYMDLRGLRSDDTAVYYCVRDRSNGAGKRYESSNWFLDLWGRGTLVTVSS

SEQ ID NO: 48 - protein, 99 aa, Artificial Sequence; identical to SEQ ID NO: 28
  Description of Artificial Sequence Synthetic polypeptide

SEQ ID NO: 49 - protein, 128 aa, Artificial Sequence; identical to SEQ ID NO: 41
  Description of Artificial Sequence Synthetic polypeptide

SEQ ID NO: 50 - protein, 99 aa, Artificial Sequence; identical to SEQ ID NO: 40
  Description of Artificial Sequence Synthetic polypeptide

SEQ ID NO: 51 - protein, 128 aa, Artificial Sequence; identical to SEQ ID NO: 47
  Description of Artificial Sequence Synthetic polypeptide

SEQ ID NO: 52 - protein, 99 aa, Artificial Sequence; identical to SEQ ID NO: 40
  Description of Artificial Sequence Synthetic polypeptide

SEQ ID NO: 53 - protein, 128 aa, Artificial Sequence; identical to SEQ ID NO: 47
  Description of Artificial Sequence Synthetic polypeptide

SEQ ID NO: 54 - protein, 99 aa, Artificial Sequence; identical to SEQ ID NO: 40
  Description of Artificial Sequence Synthetic polypeptide

SEQ ID NO: 55 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPYKFQGRVSMTRDTSIETAYMDLRGLRSDDTAVYYCVRDRSNGAGKRYESSNWFLDLWGRGTLVTVSS

SEQ ID NO: 56 - protein, 99 aa, Artificial Sequence; identical to SEQ ID NO: 40
  Description of Artificial Sequence Synthetic polypeptide

SEQ ID NO: 57 - protein, 127 aa, Artificial Sequence
  Description of Artificial Sequence Synthetic polypeptide
ADLVQSGAVVKKPGDSVRISCEAQGYRFPDYIIHWIRRAPGQGPEWMGWMNPSYGQVNIPWKFQGRVSMTRDTSIETAFLDLRGLKSDDTAVYYCVRDRSNGSGKRFESSNWFLDLWGRGTAVTIQS

SEQ ID NO: 58 - protein, 99 aa, Artificial Sequence; identical to SEQ ID NO: 2
  Description of Artificial Sequence Synthetic polypeptide

SEQ ID NO: 59 - protein, 10 aa, Artificial Sequence
  Description of Artificial Sequence Synthetic peptide
GYRFPDYIIH

SEQ ID NO: 60 - protein, 17 aa, Artificial Sequence
  Description of Artificial Sequence Synthetic peptide
WMNPMGGQVNIPWKFQG

SEQ ID NO: 61 - protein, 21 aa, Artificial Sequence
  Description of Artificial Sequence Synthetic peptide
VRDRSNGSGKRFESSNWFLDL

SEQ ID NO: 62 - protein, 8 aa, Artificial Sequence
  Description of Artificial Sequence Synthetic peptide
TGTHNLVS

SEQ ID NO: 63 - protein, 7 aa, Artificial Sequence
  Description of Artificial Sequence Synthetic peptide
DFNKRPS

SEQ ID NO: 64 - protein, 5 aa, Artificial Sequence
  Description of Artificial Sequence Synthetic peptide
WAYEA

SEQ ID NO: 65 - protein, 10 aa, Artificial Sequence
  Description of Artificial Sequence Synthetic peptide
GVTFPDYIIH

SEQ ID NO: 66 - protein, 17 aa, Artificial Sequence
  Description of Artificial Sequence Synthetic peptide
WMNPMGGQVNIPQKFQG

SEQ ID NO: 67 - protein, 17 aa, Artificial Sequence
  Description of Artificial Sequence Synthetic peptide
WMNPSYGQVNIPWKFQG

SEQ ID NO: 68 - protein, 21 aa, Artificial Sequence
  Description of Artificial Sequence Synthetic peptide
VRDRSNGAGKRFESSNWFLDL

SEQ ID NO: 69 - protein, 21 aa, Artificial Sequence
  Description of Artificial Sequence Synthetic peptide
VRDRGDGSRRHFDSSNWFLDL

SEQ ID NO: 70 - protein, 5 aa, Artificial Sequence
  Description of Artificial Sequence Synthetic peptide
WAYEN

SEQ ID NO: 71 - protein, 128 aa, Artificial Sequence
  Description of Artificial Sequence Synthetic polypeptide
QVDLVQSGAVVKKPGDSVRVSCEAQGYRFPDYIIHWIRRAPGQGPEWMGWMNPMGGQVNIPRKFQGRVSMTRDTSIETAYMDLRGLRSDDTAVYYCVRDRSNGAGKRFESSNWFLDLWGRGTLVTVSS

SEQ ID NO: 72 - protein, 99 aa, Artificial Sequence; identical to SEQ ID NO: 28
  Description of Artificial Sequence Synthetic polypeptide

SEQ ID NO: 73 - protein, 128 aa, Artificial Sequence; identical to SEQ ID NO: 55
  Description of Artificial Sequence Synthetic polypeptide

SEQ ID NO: 74 - protein, 99 aa, Artificial Sequence; identical to SEQ ID NO: 28
  Description of Artificial Sequence Synthetic polypeptide

SEQ ID NO: 75 - protein, 98 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 76 - protein, 10 aa, Artificial Sequence
  Description of Artificial Sequence Synthetic peptide
YYYGSGSYYN

SEQ ID NO: 77 - protein, 17 aa, Artificial Sequence
  Description of Artificial Sequence Synthetic peptide
YWYFDLWGRGTLVTVSS

SEQ ID NO: 78 - protein, 128 aa, Artificial Sequence
  Description of Artificial Sequence Synthetic polypeptide
AGLMQSGAVMKNSGASVRVSCQADGYDFIDYVIHWFRQRRGEGLEWLGWMNPSGGGTNYPRPFQGKVTMTRDTSTETAYLDVRGLTYDDTAVYYCVRDRANGSGRRRFESVNWFLDLWGRGTQITVVS

SEQ ID NO: 79 - protein, 128 aa, Artificial Sequence
  Description of Artificial Sequence Synthetic polypeptide
SAELVQSGAVVKKPGTSVKVSCQAYGYTFTDYLIHWLRQAPGQGLEWMGWMNPVYGQVNYAQNFQGRVSMTRDIYRETAFLEVRDLKTDDTGTYYCVRDTGDGSRRHFDSINWFLDLWGRGTWIRVAP

SEQ ID NO: 80 - protein, 100 aa, Artificial Sequence
  Description of Artificial Sequence Synthetic polypeptide
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTFH

SEQ ID NO: 81 - protein, 12 aa, Artificial Sequence
  Description of Artificial Sequence Synthetic peptide
WVFGGGTKLTVL

SEQ ID NO: 82 - protein, 99 aa, Artificial Sequence
  Description of Artificial Sequence Synthetic polypeptide
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVL

SEQ ID NO: 83 - protein, 99 aa, Artificial Sequence
  Description of Artificial Sequence Synthetic polypeptide
QCVLTQPRSVSGSPGQSVTISCTGTHNYVSWCQHHPGNAPKLLLYDFDKRPSGISDRFSGSRSGNTASLTISGLQPEDEADYFCWAFEAFGGGTKVLVL

(19) United States
(12) Patent Application Publication
(10) Pub. No.: US 2021/0040184 A1
(43) Pub. Date: Feb. 11, 2021

(54) ANTI-HIV ANTIBODIES

(71) Applicants: ATRECA, INC., South San Francisco, CA (US); UNIVERSITY OF MARYLAND, BALTIMORE, Baltimore, MD (US); The United States of America as Represented by the Department of Veterans Affairs, Washington, DC (US)

(72) Inventors: Guy L. CAVET, Burlingame, CA (US); Anthony Devico, Alexandria, VA (US); Dongkyoon Kim, Redwood City, CA (US); George K. Lewis, Baltimore, MD (US); Shaun M. Lippow, S. San Francisco, CA (US); Mohammad Sajadi, Cockeysville, MD (US); Katherine Williams, S. San Francisco, CA (US)

(21) Appl. No.: 17/015,287

(22) Filed: Sep. 9, 2020

Related U.S. Application Data
(63) Continuation of application No. PCT/US2019/021486, filed on Mar. 8, 2019.
(60) Provisional application No. 62/641,219, filed on Mar. 9, 2018.
(60) Provisional application No. 62/658,237, filed on Apr. 16, 2018.

(51) Int. Cl. C07K 16/10

(52) U.S. Cl. CPC C07K 16/1045; C07K 2317/76; C07K 2317/565

(57) ABSTRACT

Provided herein are anti-HIV antibodies, compositions comprising such antibodies, and methods of producing the antibodies. Additionally provided are methods of treating or preventing HIV infection, or a complication of HIV infection, using the anti-HIV antibodies.

### CROSS-REFERENCE TO RELATED APPLICATIONS

This application is a continuation of International PCT application no. PCT/US2019/021486, filed Mar. 8, 2019, which claims priority benefit of U.S. provisional patent application No. 62/641,219, filed Mar. 9, 2018 and U.S. provisional patent application No. 62/658,237, filed Apr. 16, 2018, each of which is herein incorporated by reference for all purposes.

### SEQUENCE LISTING

The instant application contains a Sequence Listing which has been submitted electronically in ASCII format and is hereby incorporated by reference in its entirety. Said ASCII copy, created on Sep. 8, 2020, is named SEQTXT_097519_1210180.TXT and is 80,078 bytes in size.

### BACKGROUND OF THE INVENTION

Analysis of HIV-infected individuals has led to discovery of hundreds of antibodies active against many different HIV strains. Various active anti-HIV antibodies identified to date include those that target five major sites of vulnerability on the virus, including the CD4 binding site, the V1-V2 apex, V3 glycans, the membrane proximal external region (MPER) and the gp120-gp41 interface. A lineage of highly active antibodies from a donor has recently been characterized. This lineage comprises multiple antibodies, including antibody L1A2.

### BRIEF SUMMARY OF SOME ASPECTS OF THE INVENTION

The present disclosure provides variants of an antibody L1A2. In some embodiments, the variants have broadly neutralizing activity. In some embodiments, the variants exhibit reduced immunogenicity and/or enhanced production properties compared to L1A2. Thus, in some aspects, provided herein is an anti-HIV antibody comprising a heavy chain variable (VH) region and a light chain variable (VL) region, wherein: (a) the VH region comprises at least one substitution in a CDR1 sequence, a CDR2 sequence, or a CDR3 sequence, wherein the CDR1 sequence comprises ²⁵GYRFPDYIIH³⁴ (SEQ ID NO: 59), the CDR2 sequence comprises ⁴⁹WMNPMGGQVNIPWKFQG⁶⁵ (SEQ ID NO: 60), and the CDR3 sequence comprises ⁹⁶VRDRSNGSGKRFESSNWFLDL¹¹⁶ (SEQ ID NO: 61), as numbered with reference to SEQ ID NO:1; and wherein the at least one substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; N, R, Q, S, or A at position 62, D, D, A, or Q at position 101; W, A, or N at position 103; Q, S, or A at positions 105; Q, S, or A at position 106; Y at position 107; and Y or F at position 112; and (b) the VL region comprises: (i) a CDR1 sequence comprising ²³TGTHNLVS³⁰ (SEQ ID NO: 62), a CDR2 sequence comprising ⁴⁶DFNKRPS⁵² (SEQ ID NO: 63), and a CDR3 sequence comprising ⁸⁵WAYEA⁸⁹ (SEQ ID NO: 64) as numbered with reference to SEQ ID NO:2; or (ii) at least one substitution in the CDR1 sequence, CDR2 sequence, or CDR3 sequence, wherein the at least one substitution is selected from the group consisting of Y at position 28; Q, S, or A at position 49; Q, S, or A at position 50; F or Y at position 85; and N at position 89. In another aspect, provided herein is an anti-HIV antibody comprising a heavy chain variable (VH) region and a light chain variable (VL) region, wherein: (a) the VH region comprises: (i) a CDR1 sequence comprising ²⁵GYRFPDYIIH³⁴ (SEQ ID NO: 59), a CDR2 sequence comprising ⁴⁹WMNPMGGQVNIPWKFQG⁶⁵ (SEQ ID NO: 60), and a CDR3 sequence comprising ⁹⁶VRDRSNGSGKRFESSNWFLDL¹¹⁶ (SEQ ID NO: 61); or (ii) at least one substitution in the CDR1 sequence, the CDR2 sequence, or the CDR3 sequence, wherein the at least one substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; N, R, Q, S, or A at position 62, D, D, A, or Q at position 101; W, A, or N at position 103; Q, S, or A at positions 105; Q, S, or A at position 106; Y at position 107; and Y or F at position 112; and (b) the VL region comprises at least one substitution in a CDR1 sequence, a CDR2 sequence, or a CDR3 sequence, wherein the CDR1 sequence comprises ²³TGTHNLVS³⁰ (SEQ ID NO: 62), the CDR2 sequence comprises ⁴⁶DFNKRPS⁵² (SEQ ID NO: 63), and the CDR3 sequence comprises ⁸⁵WAYEA⁸⁹ (SEQ ID NO: 64) as numbered with reference to SEQ ID NO:2; and wherein the at least one substitution in the CDR1 sequence, CDR2 sequence, or CDR3 sequence, wherein the at least one substitution is selected from the group consisting of Y at position 28; Q, S, or A at position 49; Q, S, or A at position 50; F or Y at position 85; and N at position 89. In some embodiments the VH region of an antibody as described in this paragraph comprises at least one of the following, as numbered with reference to SEQ ID NO:1: V at position 1, Q at position 2, Eat position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and/or the VL region comprises at least one of the following, as numbered with reference to SEQ ID NO:2: G at position 12; Y at position 28; Y, A, V, L, or I at position 32; Q at position 34; H at position 35; K at positon 38; M at position 43; K at position 62; N at position 65; S at position 72; A at position 76; E at position 77; E at position 79; D at position 81; or Y at position 83. In additional embodiments, the VH region has at least 70% identity to SEQ ID NO:1; and/or; the VL region has at least 70% identity to SEQ ID NO:2 In some embodiments, the VH region has at least 80% identity to SEQ ID NO:1; and/or the VL region has at least 80% identity to SEQ ID NO:2. In some embodiments, the VH region has at least 90% identity to SEQ ID NO:1; and; the VL region has at least 90% identity to SEQ ID NO:2. In further embodiments the VH region has at least 95% identity to SEQ ID NO:1; and/or the VL region has at least 95% identity to SEQ ID NO:2.

In another aspect, provided herein is an anti-HIV antibody comprising a heavy chain variable (VH) region and a light chain variable (VL) region, wherein: (i) the VH region has at least 70% identity to SEQ ID NO:1 and comprises at least one of the following substitutions as determined with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and (ii) the VL region comprises the amino acid sequence of SEQ ID NO:2; or an amino acid sequence having at least 70% identity to SEQ ID NO:2 and at least one of the following substitutions as determined with reference to SEQ ID NO:2: Y at position 28; Q, S, or A at position 49; Q, S, or A at position 50; F or Y at position 85; or N at position 8.

In a further aspect, provided herein is an anti-HIV antibody comprising a heavy chain variable (VH) region and a light chain variable (VL) region, wherein: (a)(i) the VH region comprises the amino acid sequence of SEQ ID NO:1; or (ii) the VH region has at least 70% identity to SEQ ID NO:1 and comprises at least one of the following substitutions as determined with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and (b) the VL region comprises an amino acid sequence having at least 70% identity to SEQ ID NO:2 and at least one of the following substitutions as determined with reference to SEQ ID NO:2: Y at position 28; Q, S, or A at position 49; Q, S, or A at position 50; F or Y at position 85; or N at position 8. In some embodiments, the VH comprises an amino acid sequence having at least 80% identity to SEQ ID NO:1; and/or the VL region comprises an amino acid sequence having at least 80% identity to SEQ ID NO:2. In other embodiments, the VH comprises an amino acid sequence having at least 90% identity to SEQ ID NO:1; and/or the VL region comprises an amino acid sequence having at least 90% identity to SEQ ID NO:2.

In a further aspect, provided herein is an anti-HIV antibody comprising a VH region and VL region, wherein the VH region has at least 90% identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region has at least 90% identity to SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58. In some embodiments, the VH region has at least 95% identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region has at least 95% identity to SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58. In other embodiments, the VH region comprises an amino acid sequence SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region comprises an amino acid sequence SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58. In some embodiments, the antibody comprises: a VH region comprising amino acid sequence SEQ ID NO:3 and a VL region comprising amino acid sequence SEQ ID NO:4; a VH region comprising amino acid sequence SEQ ID NO:5 and a VL region comprising amino acid sequence SEQ ID NO:6; a VH region comprising amino acid sequence SEQ ID NO:7 and a VL region comprising amino acid sequence SEQ ID NO:8; a VH region comprising amino acid sequence SEQ ID NO:9 and a VL region comprising amino acid sequence SEQ ID NO:10; a VH region comprising amino acid sequence SEQ ID NO:11 and a VL region comprising amino acid sequence SEQ ID NO:12; a VH region comprising amino acid sequence SEQ ID NO:13 and a VL region comprising amino acid sequence SEQ ID NO:14; a VH region comprising amino acid sequence SEQ ID NO:15 and a VL region comprising amino acid sequence SEQ ID NO:16; a VH region comprising amino acid sequence SEQ ID NO:17 and a VL region comprising amino acid sequence SEQ ID NO:18; a VH region comprising amino acid sequence SEQ ID NO:19 and a VL region comprising amino acid sequence SEQ ID NO:20; a VH region comprising amino acid sequence SEQ ID NO:21 and a VL region comprising amino acid sequence SEQ ID NO:22; a VH region comprising amino acid sequence SEQ ID NO:23 and a VL region comprising amino acid sequence SEQ ID NO:24; or a VH region comprising amino acid sequence SEQ ID NO:25 and a VL region comprising amino acid sequence SEQ ID NO:26. In some embodiments, the antibody comprises a VH region comprising amino acid sequence SEQ ID NO:27 and a VL region comprising amino acid sequence SEQ ID NO:28; a VH region comprising amino acid sequence SEQ ID NO:29 and a VL region comprising amino acid sequence SEQ ID NO:30; a VH region comprising amino acid sequence SEQ ID NO:31 and a VL region comprising amino acid sequence SEQ ID NO:32; a VH region comprising amino acid sequence SEQ ID NO:33 and a VL region comprising amino acid sequence SEQ ID NO:34; a VH region comprising amino acid sequence SEQ ID NO:35 and a VL region comprising amino acid sequence SEQ ID NO:36; a VH region comprising amino acid sequence SEQ ID NO:27 and a VL region comprising amino acid sequence SEQ ID NO:38; a VH region comprising amino acid sequence SEQ ID NO:39 and a VL region comprising amino acid sequence SEQ ID NO:40; a VH region comprising amino acid sequence SEQ ID NO:41 and a VL region comprising amino acid sequence SEQ ID NO:42; a VH region comprising amino acid sequence SEQ ID NO:43 and a VL region comprising amino acid sequence SEQ ID NO:44; a VH region comprising amino acid sequence SEQ ID NO:45 and a VL region comprising amino acid sequence SEQ ID NO:46; a VH region comprising amino acid sequence SEQ ID NO:47 and a VL region comprising amino acid sequence SEQ ID NO:48; a VH region comprising amino acid sequence SEQ ID NO:49 and a VL region comprising amino acid sequence SEQ ID NO:50; a VH region comprising amino acid sequence SEQ ID NO:51 and a VL region comprising amino acid sequence SEQ ID NO:52; a VH region comprising amino acid sequence SEQ ID NO:53 and a VL region comprising amino acid sequence SEQ ID NO:54; a VH region comprising amino acid sequence SEQ ID NO:55 and a VL region comprising amino acid sequence SEQ ID NO:56, or a VH region comprising amino acid sequence SEQ ID NO:57 and a VL region comprising amino acid sequence SEQ ID NO:58.

In additional aspects, provided herein is an expression vector comprising a polynucleotide encoding the VH region and/or the VL region of any one of the anti-HIV antibodies described in this section; and a host cell that comprises such an expression vector. In some embodiments, provided herein is a host cell comprising a polynucleotide that encodes the VH region and/or the VL region of any one of the preceding paragraphs in this section.

In a further aspect, provided herein is a method of treating or preventing an HIV infection, the method comprising administering any one of the anti-HIV antibodies as described in this section to a patient that is infected with an HIV virus, or is at risk of infection of with an HIV virus. In some embodiments, the method further comprises administering a latency reversing agent.

### BRIEF DESCRIPTION OF THE DRAWINGS

FIGS. 1A and 1B. Alignment of NVS49 lineage L1 antibodies to antibody L1A2.

FIGS. 1A and 1B discloses SEQ ID NOS 1, 75-79, 2, and 80-83, respectively, in order of appearance.

### DETAILED DESCRIPTION OF THE DISCLOSURE

### Terminology

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

As used herein, the terms, “HIV antibody” and “anti-HIV antibody” are used synonymously to refer to an antibody that binds to an HIV antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, a V_{H} CDR3 (HCDR3) is located in the variable domain of the heavy chain of the antibody in which it is found, whereas a V_{L} CDR3 (LCDR3) is the CDR3 from the variable domain of the light chain of the antibody in which it is found. The term “CDR” is used interchangeably with “HVR” when referring to CDR sequences.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, Md. (1991)). Chothia CDRs are determined as defined by Chothia (see, e.g., Chothia and Lesk J. Mol. Biol. 196:901-917 (1987)).

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal, et al., Mol Immunol 30:105-108, 1993).

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵M, less than 10⁻⁵M, less than 5×10⁻⁶ M, less than 10⁻⁶ M, less than 5×10⁻⁷M, less than 10⁻⁷M, less than 5×10⁻⁸M, less than 10⁻⁸M, less than 5×10⁻⁹ M, less than 10⁻⁹ M, less than 5×10⁻¹⁰ M, less than 10⁻¹⁰ M, less than 5×10⁻¹¹ M, less than 10⁻¹¹, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a bivalent antibody. In the context of the present invention, an “improved” K_{D} refers to a lower K_{D}. In some embodiments, an antibody of the present disclosure has a K_{D} of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹ M, less than 10⁻⁹ M, less than 5×10⁻¹⁰ M, less than 10⁻¹⁰ M, less than 5×10⁻¹¹ M, less than 10⁻¹¹M, less than 5×10⁻¹² M, less than 10⁻¹²M, less than 5×10⁻¹³ M, less than 10⁻¹³M, less than 5×10⁻¹⁴ M, less than 10⁻¹⁴M, less than 5×10⁻¹⁵M, or less than 10⁻¹⁵M or lower as measured as a monovalent antibody, such as a monovalent Fab. In some embodiments, an anti-HIV antibody of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, an anti-HIV antibody of the present disclosure has K_{D} of greater than 100 pM, e.g., in the range of 100-1000 pM or 500-1000 pM when measured by surface plasmon resonance analysis using a biosensor system such as a Biacore® system performed at 37° C.

The term “monovalent molecule” as used herein refers to a molecule that has one antigen-binding site, e.g., a Fab or scFv.

The term “bivalent molecule” as used herein refers to a molecule that has two antigen-binding sites. In some embodiments, a bivalent molecule of the present invention is a bivalent antibody or a bivalent fragment thereof. In some embodiments, a bivalent molecule of the present invention is a bivalent antibody. In some embodiments, a bivalent molecule of the present invention is an IgG. In general monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. Preferably both antigen-binding sites of a bivalent molecule share the same antigen specificity.

The term “valency” as used herein refers to the number of different binding sites of an antibody for an antigen. A monovalent antibody comprises one binding site for an antigen. A bivalent antibody comprises two binding sites for the same antigen.

The term “avidity” as used herein in the context of antibody binding to an antigen refers to the combined binding strength of multiple binding sites of the antibody. Thus, “bivalent avidity” refers to the combined strength of two binding sites.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this invention, the antibody binds to HIV gp120.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a V_{H} region polypeptide “corresponds to” an amino acid in the V_{H} region of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions and/or insertions. In the present invention, a “variant” with reference to the sequences described in the “Anti-HIV Antibody Variants” section refers to a engineered sequence, rather than a naturally occurring sequence. The term “sibling” as used herein with respect to an antibody refers to a naturally occurring antibody that exhibits similarity in aspects such as the same HV germline, same or similar H-CDR3 length, same LV germline, and same or similar L-CDR3 length, that may have arisen from the same ancestral naïve B-cell.

### Anti-HIV Antibody Variants

Provided herein are anti-HIV antibody variants of antibodies derived from a patient. In some embodiments, an anti-HIV antibody variant exhibits broadly neutralizing activity. In some embodiments, the variants exhibit one or more improved properties to the anti-HIV antibody compared to the naturally occurring counterpart from which it is derived. In some embodiments, an anti-HIV antibody of the present disclosure comprises modifications compared to the naturally occurring antibody L1A2 that provides improved pharmacokinetic properties, increased serum stability, increased binding affinity, and/or neutralization of HIV compared to the naturally occurring L1A2 antibody. In some embodiments, a variant antibody as described herein exhibits reduced immunogenicity and/or increased efficiency of manufacture compared to the naturally occurring antibody L1A2. In some embodiments, a variant anti-HIV antibody having at least one modification, e.g., substitution, relative to the native L1A2 variable heavy chain or light chain sequence as described herein has improved development properties, e.g., decreased heterogeneity, increased yield, increased stability, improved net charges to improve pharmacokinetics, and or/reduced immunogenicity. In some embodiments, a V_{H} region or a V_{L} region of such an antibody has at least two, three, four, five, or six, or more modifications, e.g., substitutions, as described herein. In some embodiments, a variant anti-HIV antibody of the invention has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both variable regions, compared to L1A2. In some embodiments, such a variant has broadly neutralizing activity.

The variable region sequences of L1A2 are provided in Table 1:

| Name | V_{H} amino acid sequence | V_{L} amino acid sequence |
|---|---|---|
| NVS49-L1A2 | ADLVQSGAVVKKPGDSVRIS | QSALTQPRSVSASPGQSVTISCTGTHNLVSWCQHQ |
| | CEAQGYRFPDYIIHWIRRAP | PGRAPKLLIYDFNKRPSGVPDRFSGSGSGGTASLTI |
| | GQGPEWMGWMNPMGGQV | TGLQDDDDAEYFCWAYEAFGGGTKLTVL (SEQ |
| | NIPWKFQGRVSMTRDTSIET | ID NO: 2) |
| | AFLDLRGLKSDDTAVYYCV | |
| | RDRSNGSGKRFESSNWFLDL | |
| | WGRGTAVTIQS (SEQ ID | |
| | NO: 1) | |

The heavy and light chain CDRs of L1A2 are shown in Table 2:

| | CDR1 | CDR2 | CDR3 |
|---|---|---|---|
| L1A2 VH | GYRFPDYIIH | WMNPMGGQVNIPWKFQG | VRDRSNGSGKRFESSNWFLDL |
| region | (SEQ ID NO: 59) | (SEQ ID NO: 60) | (SEQ ID NO: 61) |
| L1A2 VL | TGTHNLVS | DFNKRPS | WAYEA |
| region | (SEQ ID NO: 62) | (SEQ ID NO: 63) | (SEQ ID NO: 64) |

Position 127 of SEQ ID NO:1 and position 99 of SEQ ID NO:2 are considered to be the last amino acids of the V_{H} and V_{L} regions, respectively, according to EU index numbering. In a human IgG format (e.g., IgG1, IgG2, IgG3, or IgG4), the subsequent residue is termed the “junction codon”, and is natively encoded by the junction of the final 3′ base of the variable region gene (HJ or LJ) with the first two 5′ bases of the constant region gene (heavy or light), and exhibits amino acid variation due to variation in the final 3′ base of HJ and LJ. The human heavy chain junction codon can natively be Ala, Ser, Pro, or Thr, and is usually an Ala. The human kappa chain junction codon can natively be Arg or Gly, and is usually an Arg. The human lambda chain junction codon can natively be Gly, Ser, Arg, or Cys, and is usually a Ser or Gly.

### V_{H} Region

In some embodiments, an anti-HIV antibody of the present invention has one, two, or three CDRs of a V_{H} sequence of the antibody designated as L1A2 in Table 1; with at least one mutation in the V_{H} amino acid sequence compared to the V_{H} sequence of L1A2. In some embodiments, the V_{H} region comprises 1 or 2 substitutions relative to the CDR2 or CDR3 sequence shown in Table 2. In some embodiments, the V_{H} region has 1, 2, 3, 4, 5, or 6 substitutions relative to the CDR2 or CDR3 sequence shown in Table 2. In some embodiments, the V_{H} region comprises a CDR1 as shown in Table 2; or has 1, 2, 3, or 4 substitutions, e.g., conservative substitutions.

In some embodiments, an anti-HIV antibody of the present invention has a V_{H} that comprises a CDR2 sequence as shown in Table 2 in which one or two of positions 49, 50, 53, 61, and 62 are substituted; or in which three, four, or all five positions are substituted. In some embodiments, the CDR2 comprises one, two, or three additional substitutions, e.g., conservative substitutions. In some embodiments, the V_{H} region comprises the CDR2 sequence shown Table 2 in which position 49, 50, 53, 61, or 62 is substituted, as numbered with reference to SEQ ID NO:1, and the substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; and N, R, Q, S, or A at position 62. In some embodiments, the CDR2 comprises a substitution at position 49, 50, 53, 54, 61, or 62 as designated in the preceding sentence and 1, 2, 3, or 4 additional substitutions in the CDR2 sequence. In some embodiments, the CDR2 comprises substitutions at two of positions 49, 50, 53, 54, 61 or 63, wherein the substitutions are selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; and N, R, Q, S, or A at position 62. In some embodiments, the CDR2 comprises substitutions at three, four, or five of positions 49, 50, 53, 54, 61 or 63, wherein the substitutions are selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; and N, R, Q, S, or A at position 62. In some embodiments, the substitution is at position 49, 50, 53, 54, or 62. In some embodiments, the substitutions are at position 49, 50, 53, 54, or 62. In some embodiments, the CDR2 comprises a substitutions at position 61. In some embodiments, the CDR2 has at least 80% identity to the CDR2 sequence set forth in Table 2 and comprises at least one substitution at position 49, 50, 53, 54, or 62; wherein the substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; and N, R, Q, S, or A at position 62. In some embodiments, the CDR2 comprises a CDR2 sequence as shown in Table 2 in which positions 53 and 54 are substituted. In some embodiments, the CDR2 comprises S, V, Q, L, A G, P, I, or T at position 53; and Y, F, W, N, H, L, or I at position 54. In some embodiments, the CDR2 comprises S, V, Q, L, A G, P, I, or T at position 53; and Y, F, W, N, H, L, or I at position 54; and 1 or 2 additional substitutions relative to the CDR2 2 sequence shown in Table 2. In some embodiments, the CDR2 comprises S at position 53 and Y at position 54. In some embodiments, the CDR2 comprises S at position 53 and Y at position 54; and 1 or 2 additional substitutions; relative to the CDR2 sequence shown in Table 2.

In some embodiments, an anti-HIV antibody of the present invention has a V_{H} that comprises a CDR3 sequence as shown in Table 2 in which one or two positions 101, 103, 105, 106, 107, or 112, as numbered with reference to SEQ ID NO:1, are substituted; or in which three, four, five, or all six positions are substituted. In some embodiments, the V_{H} region comprises the CDR3 sequence shown Table 2 in which one position 101, 103, 105, 106, 107, or 112, as numbered with reference to SEQ ID NO:1, is substituted and the substitution is selected from the group consisting of D, A, S, or Q at position 101; W, A, or N at position 103; Q, S, or A at positions 105; Q, S, or A at position 106; Y at position 107; and Y or F at position 112. In some embodiments, the CDR3 comprises a substitution at position 101, 103, 105, 107, or 112 as designated in the preceding sentence and 1, 2, 3, or 4 additional substitutions in the CDR3 sequence. In some embodiments, the CDR3 comprises substitutions at two or three of positions 101, 103, 105, 106, 107, or 112, wherein the substitutions are selected from the group consisting of D, A, S, or Q at position 101; W, A, or N at position 103; Q, S, or A at positions 105; Q, S, and A at position 106; Y at position 107; and Y or F at position 112. In some embodiments, the CDR3 comprises substitutions at four, five or all six of positions 101, 103, 105, 106, 107, or 112, wherein the substitutions are selected from the group consisting of D, A, S, or Q at position 101; W, A, or N at position 103; Q, S, or A at positions 105; Q, S, or A at position 106; Y at position 107; and Y or F at position 112. In some embodiments, the substitution is at position 112. In some embodiments, the substitution is at position 105, 106, or 107. In some embodiments, the substitution is at position 101. In some embodiments, the substitution is at position 103. In some embodiments, the CDR3 has at least 80% identity to the CDR3 sequence set forth in Table 2 and comprises at least one substitution at position 101, 103, 105, 106, 107, or 112; wherein the substitutions are selected from the group consisting of D, A, S, or Q at position 101; W, A, or N at position 103; Q, S, or A at positions 105; Q, S, or A at position 106; Y at position 107; and Y or F at position 112.

In some embodiments, an anti-HIV antibody of the present invention comprises a V_{H} region CDR2 and/or a CDR3 as described in the preceding two paragraphs and a CDR1 as shown in Table 2 or a CDR1 having 1, 2, or 3 substitutions, e.g., conservative substitutions, relative to the CDR1 of Table 1. In some embodiments, an anti-HIV antibody of the present invention comprises a V_{H} region CDR2 and/or a CDR3 as described in the preceding two paragraphs and has at least 70% identity, at least 75% identity, at least 80% identity, or at least 85% identity, at least 90% identity, or at least 95% identity to SEQ ID NO:1. In some embodiments, the V_{H} region comprises a CDR1 as shown in Table 2. In some embodiments, the V_{H} region further comprises at least one of the following, as numbered with reference to SEQ ID NO:1: V at position 1, Q at position 2, Eat position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126. In some embodiments, the V_{H} region includes an additional amino acid at the N-terminal end (position “0”), e.g., Q.

In some embodiments, an anti-HIV antibody comprises a CDR2 and/or a CDR3 as described in the previous paragraphs in this section and comprises two, three, four, or five additional amino acid changes relative to SEQ ID NO:1, but no more than thirty, or no more than thirty-five, additional changes. In some embodiments, the antibody comprises at least six, seven, eight, nine or ten additional amino changes relative to SEQ ID NO:1, but no more than thirty, or thirty-five, additional changes.

In some embodiments, an anti-HIV antibody of the present invention has at least 70% identity, at least 75% identity, at least 80% identity, or at least 85% identity, at least 90% identity, or at least 95% identity to SEQ ID NO:1 and comprises 1 or more of the following: Y or F at position 49; I, Q, L, S, or A at position 50; S, V, Q, L, A G, P, I, or T at position 53; Y, F, W, N, H, L, or I at position 54; Q, Y, or F at position 61; N, R, Q, S, or A at position 62; D, A, S, or Q at position 101; W, A, or N at position 103; Q, S, or A at positions 105; Q, S, or A at position 106; Y at position 107; Y or F at position 112; V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126. In some embodiments, the V_{H} region includes an additional amino acid at the N-terminal end (position “0”), e.g., Q.

### V_{L} Region

In some embodiments, an anti-HIV antibody of the present invention has at least one, at least two, or three CDRs of a V_{L} sequence of the antibody L1 A2 shown in Table 1; and at least one mutation, e.g., a deletion, substitution, or addition, in the amino acid sequence of the V_{L} region of the antibody compared to the L1A2 V_{L} sequence. In some embodiments, the CDR1 comprises one substitution compared to the CDR1 of Table 2. In some embodiments, the CDR2 comprises 1 or 2 substitutions relative to the CDR2 sequence of Table 2. In some embodiments, the CDR3 comprises 1 or 2 substitutions relative to the CDR3 sequence of Table 2.

In some embodiments, an anti-HIV antibody of the present invention has a V_{L} that comprises a CDR1 sequence as shown in Table 2 in which position 28 is substituted. In some embodiments, position 28 is Y. In some embodiments, the CDR1 comprises 1 or 2 additional substitutions, e.g., conservative substitutions, relative to the CDR1 sequence set forth in Table 2. In some embodiments, an anti-HIV antibody of the present disclosure comprises a V_{L} region comprising a CDR2 sequence as shown in Table 2 in which position 49 and/or position 50 is substituted. In some embodiments, position 49 and/or position 50 is Q, S, or A. In some embodiments, the CDR2 comprises 1 or 2 additional substitutions, e.g., conservative substitutions, relative to the CDR2 sequence as shown in Table 2. In some embodiments, an anti-HIV antibody of the present disclosure comprises a V_{L} region comprising a CDR3 sequence as shown in Table 2 in which position 85 and/or positon 89 is substituted. In some embodiments position 85 is F or Y; and/or position 89 is N. In some embodiments, the CDR3 comprises 1 or 2 additional substitutions, e.g., conservative substitutions, relative to the sequence shown in Table 2.

In some embodiments, an anti-HIV antibody of the present invention comprises a V_{L} region CDR1, CDR2, and/or a CDR3 as described in the previous paragraphs. In some embodiments, one or two of CDR1 and CDR2 are the native sequence shown in Table 2. In some embodiments, the V_{L} region has at least 70% identity, at least 75% identity, at least 80% identity, or at least 85% identity, at least 90% identity, or at least 95% identity to SEQ ID NO:2. In some embodiments, an antibody having a substitution in a V_{L} CDR1, CDR2, and/or CDR3 further comprises at least one of the following, as numbered with reference to SEQ ID NO:2: G at position 12; Y at position 28; Y, A, V, L, or I at position 32; Q at position 34; H at position 35; K at position 38; M at position 43; K at position 62; N at position 65; S at position 72; A at position 76; E at position 77; E at position 79; D at position 81; or Y at position 83.

In some embodiments, an anti-HIV antibody comprises a V_{L} region CDR1, CDR2 and/or a CDR3 as described in the previous paragraphs in this section and comprises two, three, four, or five additional amino acid changes relative to SEQ ID NO:2, but no more than thirty additional changes. In some embodiments, the antibody comprises at least six, seven, eight, nine or ten additional amino changes relative to SEQ ID NO:2, but no more than, but no more than twenty five, or no more than thirty, additional changes.

In some embodiments, an anti-HIV antibody of the present invention comprises a V_{L} region having at least 70% identity, at least 75% identity, at least 80% identity, or at least 85% identity, at least 90% identity, or at least 95% identity to SEQ ID NO:2; and having at least one of the following: Q, S, or A at position 49; Q, S, or A at position 50; F or Y at position 85; N at position 89; G at position 12; Y at position 28; Y, A, V, L, or I at position 32; Q at position 34; H at position 35; K at position 38; M at position 43; K at position 62; N at position 65; S at position 72; A at position 76; E at position 77; E at position 79; D at position 81; or Y at position 83.

### Illustrative Antibodies

In some embodiments, an anti-HIV antibody of the present invention comprises a V_{H} region and a V_{L} region as described in the preceding paragraphs in this section.

In some embodiments, provided herein anti-HIV antibodies comprising the CDR1, CDR2, and CDR3 of a V_{H} region of any one of SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51 SEQ ID NO:53, SEQ ID NO:55, or SEQ ID NO:57, or SEQ ID NO:55; or anti-HIV antibodies comprising the CDR1, CDR2, and CDR3 of a V_{L} region of any one of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58. In some embodiments, an anti-HIV antibody of the present invention comprises the six CDRs of an antibody designated as Germ6, Germ12, Germ18, Germ17, Germ23, NglycoSA, NglycoND, NglycoNDplus6, CysCA, CysCV, Hydro2, Germ12_NglycoSA_CysCA in Table 3. In some embodiments, an anti-HIV antibody of the present invention comprises the six CDRs of an antibody designated as Germ12_NglycoSA_CysCV, Germ18_NglycoSA_CysCV, Germ17_NglycoSA_CysCA, Germ17_NglycoSA_CysCV, Germ23_NglycoSA_CysCV, Germ12_NglycoSA_CysCV_H61WQ, Germ12_NglycoSA_CysCV_L89AN, Germ12_NglycoSA_CysCV_H61WY, Germ12_NglycoSA_CysCV_H61WH, Germ12_NglycoSA_CysCV_H61WH, Germ12_NglycoSA_CysCV_H107FY, Germ12_NglycoSA_CysCV_L89AN_H61WY, Germ12_NglycoSA_CysCV_L89AN_H107FY, Germ12_NglycoSA_CysCV_L89AN_H107FY, Germ12_NglycoSA_CysCV_L89AN_H61WY_H107FY, or Cd4bs_H53MS_H54GY in Table 3. In some embodiments, an anti-HIV antibody of the present invention comprises the six CDRs of an antibody designated as Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ23_NglycoSA_CysCV, or Cd4bs_H53MS_H54GY in Table 11.

In some embodiments, provided herein are anti-HIV antibodies comprising a V_{H} having at least 90% identity, or at least 95% identity, to an amino acid sequence SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57. In some embodiments, provided herein anti-HIV antibodies comprising a V_{L} having at least 90% identity, or at least 95% identity, to an amino acid sequence SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58.

In some embodiments, an anti-HIV antibody of the present invention comprises a V_{H} comprising an amino acid sequence of SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; or a V_{L} comprising an amino acid sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58. In some embodiments, an anti-HIV antibody of the present invention comprises a V_{H} comprising an amino acid sequence of SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and a V_{L} comprising an amino acid sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, or SEQ ID NO:58.

In some embodiments, an anti-HIV antibody of the present invention comprises a V_{H} having at least 85% identity, or at least 90% identity; or at least 95% identity; and a V_{L} having at least 85% identity, or at least 90% identity; or at least 95% identity to the V_{H} and V_{L} of the antibody Germ6, Germ12, Germ18, Germ17, Germ23, NglycoSA, NglycoND, NglycoNDplus6, CysCA, CysCV, Hydro2, or Germ12_NglycoSA_CysCA as designated in Table 3; or to a V_{H} and V_{L} of an antibody Germ12_NglycoSA_CysCV, Germ18_NglycoSA_CysCV, Germ17_NglycoSA_CysCA, Germ17_NglycoSA_CysCV, Germ23_NglycoSA_CysCV, Germ12_NglycoSA_CysCV_H61WQ, Germ12_NglycoSA_CysCV_L89AN, Germ12_NglycoSA_CysCV_H61WY, Germ12_NglycoSA_CysCV_H61WH, Germ12_NglycoSA_CysCV_H61WH, Germ12_NglycoSA_CysCV_H107FY, Germ12_NglycoSA_CysCV_L89AN_H61WY, Germ12_NglycoSA_CysCV_L89AN_H107FY, Germ12_NglycoSA_CysCV_L89AN_H107FY, Germ12_NglycoSA_CysCV_L89AN_H61WY_H107FY, or Cd4bs_H53MS_H54GY as designated in Table 3. In some embodiments, an anti-HIV antibody of the present invention comprises a V_{H} having at least 85% identity, or at least 90% identity; or at least 95% identity; and a V_{L} having at least 85% identity, or at least 90% identity; or at least 95% identity to the V_{H} and V_{L} of the antibody Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ23_NglycoSA_CysCV, or Cd4bs_H53MS_H54GY. In some embodiments, such an antibody has no more than ten mutations, or no more than nine mutations, no more than eight mutations, or no more than seven mutations in total in the heavy and light chain CDR sequences compared to the CDR sequences of Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ18_NglycoSA_CysCV, Germ17_NglycoSA_CysCA, Germ17_NglycoSA_CysCV, Germ23_NglycoSA_CysCV, Germ12_NglycoSA_CysCV_H61WQ, Germ12_NglycoSA_CysCV_L89AN, Germ12_NglycoSA_CysCV_H61WY, Germ12_NglycoSA_CysCV_H61WH, Germ12_NglycoSA_CysCV_H61WH, Germ12_NglycoSA_CysCV_H107FY, Germ12_NglycoSA_CysCV_L89AN_H61WY, Germ12_NglycoSA_CysCV_L89AN_H107FY, Germ12_NglycoSA_CysCV_L89AN_H107FY, or Germ12_NglycoSA_CysCV_L89AN_H61WY_H107FY, or Cd4bs_H53MS_H54GY as designated in Table 3. In some embodiments, such an antibody has no more than ten mutations, or no more than nine mutations, no more than eight mutations, or no more than seven mutations in total in the heavy and light chain CDR sequences compared to the CDR sequences of Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ23_NglycoSA_CysCV, or Cd4bs_H53MS_H54GY. In some embodiments, the antibody has six, four, three, two or one mutation in total in the heavy and light chain CDR sequences compared to the CDR sequences of Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ18_NglycoSA_CysCV, Germ17_NglycoSA_CysCA, Germ17_NglycoSA_CysCV, Germ23_NglycoSA_CysCV, Germ12_NglycoSA_CysCV_H61WQ, Germ12_NglycoSA_CysCV_L89AN, Germ12_NglycoSA_CysCV_H61WY, Germ12_NglycoSA_CysCV_H61WH, Germ12_NglycoSA_CysCV_H61WH, Germ12_NglycoSA_CysCV_H107FY, Germ12_NglycoSA_CysCV_L89AN_H61WY, Germ12_NglycoSA_CysCV_L89AN_H107FY, Germ12_NglycoSA_CysCV_L89AN_H107FY, or Germ12_NglycoSA_CysCV_L89AN_H61WY_H107FY, or Cd4bs_H53MS_H54GY as designated in Table 3. In some embodiments, the antibody has six, four, three, two or one mutation in total in the heavy and light chain CDR sequences compared to the CDR sequences of Germ6, Germ12, Germ18, Germ17, Germ23, NglycoSA, NglycoND, NglycoNDplus6, CysCA, CysCV, Hydro2, Germ12_NglycoSA_CysCA, Germ12_NglycoSA_CysCV, Germ23_NglycoSA_CysCV, or Cd4bs_H53MS_H54GY. In some embodiments, all of the mutations are substitutions relative to the corresponding sequence shown in Table 3. In some embodiments, substitutions in a V_{H} or V_{L} sequence are germline mutations, i.e., mutations to amino acid residues present in the germline sequence of origin of the V_{H} or V_{L} sequence; and/or the heavy chain CDR3 comprises the sequence motif 101N-102G-103S, but in which the S at position 103 is substituted with A, or another residue to remove the N-glycosylation motif. In some embodiments, the antibody has a V_{L} sequence comprising V, I, or L at position 32. In some embodiments, V is present at position 32 of the V_{L} region. In some embodiments, the antibody comprises a substitution, relative to SEQ ID NO:1, in the V_{H} at position 61 or position 107 that reduces hydrophobicity. In some embodiments, the antibody comprises Q, Y, H, or R at positon 61 of the V_{H} region; and/or Y at position 107 of the V_{H} region. In some embodiments, the antibody comprises a substitution, relative to SEQ ID NO:2, at position 89 of the V_{L} region. In some embodiments, the V_{L} region comprises N at position 89.

TABLE 3

| Name | VH amino acid sequence | VL amino acid sequence |
|---|---|---|
| Germ6 | ADLVQSGAVVKKPGDSVRVSCEA | QSALTQPRSVSGSPGQSVT |
| | QGYRFPDYIIHWIRRAPGQGPEWM | ISCTGTHNLVSWCQHQPG |
| | GWMNPMGGQVNIPWKFQGRVSM | RAPKLLIYDFNKRPSGVPD |
| | TRDTSIETAYLDLRGLKSDDTAVY | RFSGSGSGGTASLTISGLQ |
| | YCVRDRSNGSGKRFESSNWFLDL | DEDDAEYFCWAYEAFGG |
| | WGRGTAVTVQS (SEQ ID NO: 3) | GTKLTVL (SEQ ID NO: 4) |
| Germ12 | QVDLVQSGAVVKKPGDSVRVSCE | QSALTQPRSVSGSPGQSVT |
| | AQGYRFPDYIIHWIRRAPGQGPEW | ISCTGTHNLVSWCQHQPG |
| | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGSGKRFESSNWFL | DEDDAEYFCWAYEAFGG |
| | DLWGRGTLVTVSS (SEQ ID NO: 5) | GTKLTVL (SEQ ID NO: 6) |
| Germ18 | QVQLVQSGAEVKKPGASVKVSCE | QSALTQPRSVSGSPGQSVT |
| | ASGYRFPDYIIHWIRRAPGQGPEW | ISCTGTHNLVSWCQHQPG |
| | MGWMNPMGGQVNIPWKFQGRVS | RAPKLLIYDFNKRPSGVPD |
| | MTRDTSIETAYMELRGLRSDDTAV | RFSGSGSGGTASLTISGLQ |
| | YYCVRDRSNGSGKRFESSNWFLD | DEDDAEYFCWAYEAFGG |
| | LWGRGTLVTVSS (SEQ ID NO: 7) | GTKLTVL (SEQ ID NO: 8) |
| Germ17 | QVDLVQSGAVVKKPGDSVRVSCE | QSALTQPRSVSGSPGQSVT |
| | AQGYRFPDYIIHWIRRAPGQGPEW | ISCTGTHNLVSWCQHHPG |
| | MGWMNPMGGQVNIPWKFQGRVS | KAPKLMIYDFNKRPSGVP |
| | MTRDTSIETAYMDLRGLRSDDTA | DRFSGSGSGGTASLTISGL |
| | VYYCVRDRSNGSGKRFESSNWFL | QAEDEAEYFCWAYEAFG |
| | DLWGRGTLVTVSS (SEQ ID NO: 9) | GGTKLTVL (SEQ ID |
| | | NO: 10) |
| Germ23 | QVQLVQSGAEVKKPGASVKVSCE | QSALTQPRSVSGSPGQSVT |
| | ASGYRFPDYIIHWIRRAPGQGPEW | ISCTGTHNLVSWCQHHPG |
| | MGWMNPMGGQVNIPWKFQGRVS | KAPKLMIYDFNKRPSGVP |
| | MTRDTSIETAYMELRGLRSDDTAV | DRFSGSGSGGTASLTISGL |
| | YYCVRDRSNGSGKRFESSNWFLD | QAEDEAEYFCWAYEAFG |
| | LWGRGTLVTVSS (SEQ ID NO: 11) | GGTKLTVL (SEQ ID |
| | | NO: 12) |
| NglycoSA | ADLVQSGAVVKKPGDSVRISCEA | QSALTQPRSVSASPGQSVT |
| | QGYRFPDYIIHWIRRAPGQGPEWM | ISCTGTHNLVSWCQHQPG |
| | GWMNPMGGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRSNGAGKRFESSNWFLDL | DDDDAEYFCWAYEAFGG |
| | WGRGTAVTIQS (SEQ ID NO: 13) | GTKLTVL (SEQ ID NO: 14) |
| NglycoND | ADLVQSGAVVKKPGDSVRISCEA | QSALTQPRSVSASPGQSVT |
| | QGYRFPDYIIHWIRRAPGQGPEWM | ISCTGTHNLVSWCQHQPG |
| | GWMNPMGGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRSDGSGKRFESSNWFLDL | DDDDAEYFCWAYEAFGG |
| | WGRGTAVTIQS (SEQ ID NO: 15) | GTKLTVL (SEQ ID NO: 16) |
| NglycoNDplus6 | ADLVQSGAVVKKPGDSVRISCEA | QSALTQPRSVSASPGQSVT |
| | QGYTFPDYIIHWIRRAPGQGPEWM | ISCTGTHNLVSWCQHQPG |
| | GWMNPMGGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRGDGSRRHFDSSNWFLDL | DDDDAEYFCWAYEAFGG |
| | WGRGTAVTIQS (SEQ ID NO: 17) | GTKLTVL (SEQ ID NO: 18) |
| CysCA | ADLVQSGAVVKKPGDSVRISCEA | QSALTQPRSVSASPGQSVT |
| | QGYRFPDYIIHWIRRAPGQGPEWM | ISCTGTHNLVSWAQHQPG |
| | GWMNPMGGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRSNGSGKRFESSNWFLDL | DDDDAEYFCWAYEAFGG |
| | WGRGTAVTIQS (SEQ ID NO: 19) | GTKLTVL (SEQ ID NO: 20) |
| CysCV | ADLVQSGAVVKKPGDSVRISCEA | QSALTQPRSVSASPGQSVT |
| | QGYRFPDYIIHWIRRAPGQGPEWM | ISCTGTHNLVSWVQHQPG |
| | GWMNPMGGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRSNGSGKRFESSNWFLDL | DDDDAEYFCWAYEAFGG |
| | WGRGTAVTIQS (SEQ ID NO: 21) | GTKLTVL (SEQ ID NO: 22) |
| Hydro2 | ADLVQSGAVVKKPGDSVRISCEA | QSALTQPRSVSASPGQSVT |
| | QGYRFPDYIIHWIRRAPGQGPEWM | ISCTGTHNLVSWCQHQPG |
| | GWMNPMGGQVNIPQKFQGRVSM | RAPKLLIYDFNKRPSGVPD |
| | TRDTSIETAFLDLRGLKSDDTAVY | RFSGSGSGGTASLTITGLQ |
| | YCVRDRSNGSGKRFESSNWFLDL | DDDDAEYFCWAYENFGG |
| | WGRGTAVTIQS (SEQ ID NO: 23) | GTKLTVL (SEQ ID NO: 24) |
| Germ12_Nglyco | QVDLVQSGAVVKKPGDSVRVSCE | QSALTQPRSVSGSPGQSVT |
| SA_CysCA | AQGYRFPDYIIHWIRRAPGQGPEW | ISCTGTHNLVSWAQHQPG |
| | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 25) | GTKLTVL (SEQ ID NO: 26) |
| Germ12_Nglyco | QVDLVQSGAVVKKPGDSVRVSCE | QSALTQPRSVSGSPGQSVT |
| SA_CysCV | AQGYRFPDYIIHWIRRAPGQGPEW | ISCTGTHNLVSWVQHQPG |
| | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 27) | GTKLTVL (SEQ ID NO: 28) |
| Germ18_Nglyco | QVQLVQSGAEVKKPGASVKVSCE | QSALTQPRSVSGSPGQSVT |
| SA_CysCV | ASGYRFPDYIIHWIRRAPGQGPEW | ISCTGTHNLVSWVQHQPG |
| | MGWMNPMGGQVNIPWKFQGRVS | RAPKLLIYDFNKRPSGVPD |
| | MTRDTSIETAYMELRGLRSDDTAV | RFSGSGSGGTASLTISGLQ |
| | YYCVRDRSNGAGKRFESSNWFLD | DEDDAEYFCWAYEAFGG |
| | LWGRGTLVTVSS (SEQ ID NO: 29) | GTKLTVL (SEQ ID NO: 30) |
| Germ17_Nglyco | QVDLVQSGAVVKKPGDSVRVSCE | QSALTQPRSVSGSPGQSVT |
| SA_CysCA | AQGYRFPDYIIHWIRRAPGQGPEW | ISCTGTHNLVSWAQHHPG |
| | MGWMNPMGGQVNIPWKFQGRVS | KAPKLMIYDFNKRPSGVP |
| | MTRDTSIETAYMDLRGLRSDDTA | DRFSGSGSGGTASLTISGL |
| | VYYCVRDRSNGAGKRFESSNWFL | QAEDEAEYFCWAYEAFGG |
| | DLWGRGTLVTVSS (SEQ ID NO: 31) | GTKLTVL (SEQ ID NO: 32) |
| Germ17_Nglyco | QVDLVQSGAVVKKPGDSVRVSCE | QSALTQPRSVSGSPGQSVT |
| SA_CysCV | AQGYRFPDYIIHWIRRAPGQGPEW | ISCTGTHNLVSWVQHHPG |
| | MGWMNPMGGQVNIPWKFQGRVS | KAPKLMIYDFNKRPSGVP |
| | MTRDTSIETAYMDLRGLRSDDTA | DRFSGSGSGGTASLTISGL |
| | VYYCVRDRSNGAGKRFESSNWFL | QAEDEAEYFCWAYEAFGG |
| | DLWGRGTLVTVSS (SEQ ID NO: 33) | GTKLTVL (SEQ ID NO: 34) |
| Germ23_Nglyco | QVQLVQSGAEVKKPGASVKVSCE | QSALTQPRSVSGSPGQSVT |
| SA_CysCV | ASGYRFPDYIIHWIRRAPGQGPEW | ISCTGTHNLVSWVQHHPG |
| | MGWMNPMGGQVNIPWKFQGRVS | KAPKLMIYDFNKRPSGVP |
| | MTRDTSIETAYMELRGLRSDDTAV | DRFSGSGSGGTASLTISGL |
| | YYCVRDRSNGAGKRFESSNWFLD | QAEDEAEYFCWAYEAFGG |
| | LWGRGTLVTVSS (SEQ ID NO: 35) | GTKLTVL (SEQ ID NO: 36) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| H61WQ | MGWMNPMGGQVNIPQKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 37) | GTKLTVL (SEQ ID NO: 38) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| L89AN | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYENFGG |
| | DLWGRGTLVTVSS (SEQ ID NO: 39) | GTKLTVL (SEQ ID NO: 40) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| H61WY | MGWMNPMGGQVNIPYKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 41) | GTKLTVL (SEQ ID NO: 42) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| H61WH | MGWMNPMGGQVNIPHKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 43) | GTKLTVL (SEQ ID NO: 44) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| H61WH | MGWMNPMGGQVNIPHKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYEAFGG |
| | DLWGRGTLVTVSS (SEQ ID NO: 45) | GTKLTVL (SEQ ID NO: 46) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| H107FY | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRYESSNWFL | DEDDAEYFCWAYEAFGG |
| | DLWGRGTLVTVSS (SEQ ID NO: 47) | GTKLTVL (SEQ ID NO: 48) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| L89AN_H61WY | MGWMNPMGGQVNIPYKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRFESSNWFL | DEDDAEYFCWAYENFGG |
| | DLWGRGTLVTVSS (SEQ ID NO: 49) | GTKLTVL (SEQ ID NO: 50) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| L89AN_H107FY | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRYESSNWFL | DEDDAEYFCWAYENFGG |
| | DLWGRGTLVTVSS (SEQ ID NO: 51) | GTKLTVL (SEQ ID NO: 52) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| L89AN_H107FY | MGWMNPMGGQVNIPWKFQGRVS | RAPKLLIYDFNKRPSGVPD |
| | MTRDTSIETAYMDLRGLRSDDTA | RFSGSGSGGTASLTISGLQ |
| | VYYCVRDRSNGAGKRYESSNWFL | DEDDAEYFCWAYENFGG |
| | DLWGRGTLVTVSS (SEQ ID NO: 53) | GTKLTVL (SEQ ID NO: 54) |
| Germ12_Nglyco | QVDLVQSGAVVKKPGDSVRVSCE | QSALTQPRSVSGSPGQSVT |
| SA_CysCV_ | AQGYRFPDYIIHWIRRAPGQGPEW | ISCTGTHNLVSWVQHQPG |
| L89AN_H61WY_ | MGWMNPMGGQVNIPYKFQGRVS | RAPKLLIYDFNKRPSGVPD |
| H107FY | MTRDTSIETAYMDLRGLRSDDTA | RFSGSGSGGTASLTISGLQ |
| | VYYCVRDRSNGAGKRYESSNWFL | DEDDAEYFCWAYENFGG |
| | DLWGRGTLVTVSS (SEQ ID NO: 55) | GTKLTVL (SEQ ID NO: 56) |
| Cd4bs_H53MS_ | ADLVQSGAVVKKPGDSVRISCEA | QSALTQPRSVSASPGQSVT |
| H54GY | QGYRFPDYIIHWIRRAPGQGPEWM | ISCTGTHNLVSWCQHQPG |
| | GWMNPSYGQVNIPWKFQGRVSM | RAPKLLIYDFNKRPSGVPD |
| | TRDTSIETAFLDLRGLKSDDTAVY | RFSGSGSGGTASLTITGLQ |
| | YCVRDRSNGSGKRFESSNWFLDL | DDDDAEYFCWAYEAFGG |
| | WGRGTAVTIQS (SEQ ID NO: 57) | GTKLTVL (SEQ ID NO: 58) |

In a further aspect of the invention, an anti-HIV antibody according to any of the above embodiments is a monoclonal antibody, including a chimeric, antibody. In one embodiment, an anti-HIV antibody is an antibody fragment, e.g., a Fv, Fab, Fab′, scFv, diabody, or F(ab′)₂ fragment. In another embodiment, the antibody is a substantially full length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells (e.g. E. coli or phage), as described herein.

In some embodiments an anti-HIV antibody in accordance with the present disclosure is a in a monovalent format. In some embodiments, the anti-HIV antibody is in a fragment format, e.g., a Fv, Fab, Fab′, scFv, diabody, or F(ab′)₂ fragment.

In some embodiments, an anti-HIV antibody of the present invention is employed in a bispecific or multi-specific format. For example, in some embodiments, the antibody may be incorporated into a bispecific or multi-specific antibody that comprises a further binding domain that binds to the same or a different antigen.

In some embodiments, an antibody of the present disclosure comprises an Fc region that has effector function, e.g., exhibits antibody-dependent cellular cytotoxicity ADCC. In some embodiments, the Fc region may be an Fc region engineered to alter one or more functional properties of the antibody, such as serum half-life, complement fixation, Fc receptor binding, and/or ADCC. Furthermore, an antibody of the disclosure may be chemically modified (e.g., one or more chemical moieties can be attached to the antibody) or be modified to alter its glycosylation, again to alter one or more functional properties of the antibody. Additional modifications may also be introduced. For example, the antibody can be linked to one of a variety of polymers, for example, polyethylene glycol.

### Activity

The activity of an anti-HIV antibody variant as described herein can be assessed for binding to HIV, neutralization potency, and/or neutralization breadth. In some embodiments, effector function, e.g., ADCC, is also evaluated.

A “neutralizing anti-HIV antibody” as used herein refers to an antibody that can prevent HIV from initiating and perpetuating an infection in a host and/or in target cells in vitro. In some embodiments, the present invention provides neutralizing monoclonal human antibodies that bind to HIV gp120 polypeptide.

As used herein, “broadly neutralizing antibodies” refers to antibodies that neutralize multiple HIV-1 virus strains from diverse clades and different strains within a Glade in a neutralization assay. In some embodiments, a broadly neutralizing antibody may neutralize at least 50 or more different strains of HIV-1. In certain embodiments, the 50% inhibitory concentration of the monoclonal antibody may be less than about 0.0001 μg/ml, less than about 0.001 μg/ml, less than about 0.01 μg/ml, less than about 1 μg/ml, less than about 5 μg/ml, less than about 10 μg/ml, less than about 20 μg/ml, less than about 50 μg/ml, or less than about 100 μg/ml and is defined as the antibody concentration required to neutralize about 50% of the input virus in the neutralization assay.

Broadly neutralizing activity of an antibody can be determined by evaluating neutralization against a panel of HIV-1 viruses, which in some embodiments, includes viruses from multiple clades and circulating recombinant forms. These can include both chronic as well as transmitted/founder (T/F) viruses. Such assays can be performed using panels of appropriate HIV-1 pseudoviruses using methodology such as that described by Decamp et al., J. Virol. 88:2489-2507, 2014, Seaman et al., J Virol. 54: 1439-1452, 2010; or Hraber et al., J. Virol. 91: e00991-17, 2017. For example, an illustrative assay measures Tat-regulated luciferase reporter gene expression to quantify the reduction of virus infection in TZM-bl cells (Montefiori, et al. Methods Mol. Biol. 485:395-405, 2009; Sarzotti-Kelsoe,). The 50% inhibitory concentration (IC₅₀) is the concentration of antibody at which relative luminescence units are reduced by at least 50% as compared to infection in the absence of anti-HIV antibody, or in the presence of a negative control antibody after background is subtracted. In some embodiments, neutralizing activity can also be measured as a function of the area under the positive portion of the neutralization curve. Breadth and potency are two typical measures that may be employed to characterize an antibody's neutralizing activity. Breadth is the proportion of tested viruses with IC₅₀ scores that fall below an IC₅₀ cutoff value for neutralizing activity. Potency can be calculated using the geometric mean IC₅₀ (see, e.g., Hraber et al., J Virol. 88:12623-43, 2014; Rademeyer,). In some embodiments, an anti-HIV antibody as described herein is at least 0.25, 0.3, 0.35, 0.4, 0.45, 0.5, 0.6, 0.7, 0.8, 0.9 times as potent, is equivalently potent, or is more potent, than antibody L1A2 when activity is compared in the same assay.

In some embodiments, binding activity of a variant anti-HIV antibody as described herein to HIV Env protein can be assessed. Binding can be determined using any immunoassay, where examples using recombinant gp120 may include ELISA, SPR, or similar assays. The gp120 protein can be from various HIV strains. In some embodiments, the HIV strain is BaL. In some embodiments, HIV binding is assessed by measuring binding to an HIV Env trimer in which the trimer is expressed on the surface of cells transfected with HIV Env protein, is on the surface of infected cells, or is added to an ELISA as purified trimeric protein with or without the stabilizing SOSIP modification.

In some embodiments, binding to HIV Env protein is assessed in a competitive assay format with a reference antibody L1A2 or a reference antibody having the variable regions of L1A2. In some embodiments, a variant anti-HIV antibody in accordance with the present disclosure may block binding of the reference antibody in a competition assay by about 50% or more.

Anti-HIV antibodies of the present disclosure may also be evaluated in various assays for their ability to mediate FcR-dependent activity. Such assays are routine in the art. In some embodiments, antibody-dependent cellular cytotoxicity (ADCC) is measured. In some embodiments, antibody-dependent cellular viral inhibition (ADCVI) is measured. For example, ADCC can be measured by quantifying the destruction of Env-coated or HIV-infected fluorescent cells driven by the addition of either PBMCs or specific effector cell populations such as NK cells. In such an analysis, ADCC activity is reported as a reduction in percent of Env-coated or HIV-infected cells in the presence and absence of anti-HIV antibodies and effector cells. ADCVI is measured by quantifying the amount of virus produced by infected cells in the presence and absence of anti-HIV antibody and PBMCs. ADCVI is frequently reported as a reduction in p24 measured in the cellular supernatant. In some embodiments, an antibody of the present disclosure has enhanced ADCC and/or ADCVI activity compared to antibody L1A2 when the antibodies are assayed in a human IgG1 isotype format.

### Generation of Antibodies

HIV antibodies as disclosed herein are commonly produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the invention, provided herein are isolated nucleic acids encoding a V_{H} and/or V_{L} region, or fragment thereof, of any of the anti-HIV antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the anti-HIV antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

In a further aspect, the invention provides a method of making an anti-HIV antibody as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing an anti-HIV antibody as described herein include both prokaryotic or eukaryotic cells. For example, anti-HIV antibodies may be produced in bacteria, in particular when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing anti-HIVantibodies of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59, 1977; baby hamster kidney cells (BHK); mouse sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express anti-HIV antibodies. Other useful mammalian host cell lines include Chinese hamster ovary (CHO) cells, including DHFR-CHO cells (Urlaub et al., Proc. Natl. Acad. Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268, 2003.

A host cell transfected with an expression vector encoding an anti-HIV antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, provided herein is a method of generating variants of an anti-HIV antibody as disclosed herein. Thus, for example, a construct encoding a variant L1A2 V_{H} CDR3 as described in the “anti-HIV Antibody Variant” section can be additionally modified and the V_{H} region encoded by the additionally modified construct can be tested for gp120 binding activity and/or neutralizing activity in the context of a V_{H} region comprising the native CDR1 and CDR2, or a variant CDR1 or CDR2, as described herein that is paired with a native or variant L1A2 V_{L} region as described herein. Similarly, a construct encoding a variant L1A2 V_{L} CDR3 as described in the “anti-HIV Antibody Variant” section can be additionally modified and the V_{L} region encoded by the additionally modified construct can be tested for gp120 binding activity and/or neutralizing activity in the context of a V_{L} region comprising the native CDR1 and CDR2, or a variant CDR1 or CDR2, as described herein that is paired with a native or variant L1A2 V_{H} region as described herein. Such an analysis can also be performed with other CDRs or framework regions and an antibody having the desired activity can then be selected.

### Anti-HIV Antibody Conjugates

In a further aspect, an anti-HIV antibody of the present invention may be conjugated or linked to therapeutic and/or imaging/detectable moieties. For example, the anti-HIV antibody may be conjugated to a detectable marker, a toxin, or a therapeutic agent. Methods for conjugating or linking antibodies are well known in the art. The moiety may be linked to the antibody covalently or by non-covalent linkages.

In some embodiments, the antibody is conjugated to cytotoxic moiety or other moiety that inhibits cell proliferation. In some embodiments, the antibody is conjugated to a cytotoxic agent including, but not limited to, a ricin A chain, doxorubicin, daunorubicin, a maytansinoid, taxol, ethidium bromide, mitomycin, etoposide, tenoposide, vincristine, vinblastine, colchicine, dihydroxy anthracin dione, actinomycin, a diphtheria toxin, extotoxin A from Pseudomonas, Pseudomonas exotoxin (PE) A, PE40, abrin, abrin A chain, modeccin A chain, alpha sarcin, gelonin, mitogellin, restrictocin, cobran venom factor, a ribonuclease, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolites, a dichloroethylsulfide derivative, a protein production inhibitor, a ribosome inhibitor, or an inducer of apoptosis.

In some embodiments, the antibody may be linked to radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### Pharmaceutical Compositions

In a further aspect, provided herein are pharmaceutical compositions for administration of an anti-HIV antibody of the present invention to a mammalian subject, preferably a human or non-human primate subject, that is infected with HIV or is at risk of HIV infection, in an amount and according to a schedule sufficient to prevent HIV infection or reduce viral load in the subject. Such compositions may comprise an anti-HIV antibody or a polynucleotide encoding the antibody, and a pharmaceutically acceptable diluent or carrier. In some embodiments, the polynucleotide encoding the antibody may be contained in a plasmid vector for delivery, or a viral vector. In some embodiments, the pharmaceutical composition comprises a therapeutically effective amount of the antibody. As used herein, a “therapeutically effective dose” or a “therapeutically effective amount” refers to an amount sufficient to prevent, cure, or at least partially arrest HIV infection or symptoms of HIV infection and its complications. A therapeutically effective dose can be determined by monitoring a patient's response to therapy. Typical benchmarks indicative of a therapeutically effective dose include amelioration of symptoms of the disease in the patient, including, for example, reduction in viral load and increases in CD4+ lymphocyte numbers. Amounts effective for this use will depend upon the severity of the disease and the general state of the patient's health, including other factors such as age, weight, gender, administration route, etc. Single or multiple administrations of the antibody will be dependent on the dosage and frequency as required and tolerated by the patient.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In particular embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The pharmaceutical composition can be formulated for any suitable route of administration, including for example, parenteral, intrapulmonary, intranasal, or local administration. Parenteral administration can include intramuscular, intravenous, intraarterial, intraperitoneal, oral or subcutaneous administration. In certain embodiments, the pharmaceutical composition is formulated for intravenous administration and has a concentration of antibody of 10-100 mg/ml, 10-50 mg/ml, 20 to 40 mg/ml, or about 30 mg/ml. In certain embodiments, the pharmaceutical composition is formulated for subcutaneous injection and has a concentration of antibody of 50-500 mg/ml, 50-250 mg/ml, or 100 to 150 mg/ml, and a viscosity less than 50 cP, less than 30 cP, less than 20 cP, or about 10 cP. In some embodiments, the pharmaceutical compositions are liquids or solids. In particular embodiments, the pharmaceutical compositions are formulated for parenteral, e.g., intravenous, subcutaneous, or oral administration.

The formulation of and delivery methods of pharmaceutical compositions will generally be adapted according to the site and the disease to be treated. Formulations include those in which the antibody is encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays.

In some embodiments, e.g., for parenteral administration, the antibodies or antigen-binding fragments thereof are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate may also be used.

The dose and dosage regimen depends upon a variety of factors readily determined by a physician, such as the nature of the infection, the characteristics of the subject, and the subject's history. In particular embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

### Methods of Treating or Preventing HIV Infection

In a further aspect, provided herein are methods of treating and/or preventing an HIV infection or complication of an HIV infection, the method comprising administering to a subject, e.g., a human or non-human primate, in need thereof an effective amount of an anti-HIV antibody as described herein, or a polynucleotide encoding such an antibody. In some embodiments, the antibody is administered to an individual at risk of acquiring an HIV infection. In some embodiments, the antibody is administered to a patient who has acquired immune deficiency syndrome (AIDS). In some embodiments, the subject is a virologically suppressed HIV-infected mammal, such as a human or non-human primate, while in other embodiments, the subject is a treatment-naive HIV-infected mammal. In certain embodiments, a treatment-naive subject has a viral load between 10³ and 10⁵ copies/ml, and in certain embodiments, a virologically suppressed subject has a viral load <50 copies/ml. In some embodiments, the subject is a human. In certain embodiments, the subject has been diagnosed with an HIV, e.g., HIV-1 or HIV-2, infection or a related disease or disorder, e.g., AIDS, or is considered at risk for contracting an HIV, e.g., HIV-1 or HIV-2, infection and/or developing a related disease or disorder, e.g., AIDS. Subjects at risk for HIV infection include individuals who have come into contact with an infected person or who have been exposed to HIV in some other way. Administration of the antibody can occur prior to exposure such that infection or disease is prevented, or can be administered following infection to prevent, delay, and/or reduce manifestation of symptoms characteristic of HIV-related disease or disorders.

The present invention further provides methods for preventing or inhibiting an increase in HIV virus titer, virus replication, virus proliferation or an amount of an HIV viral RNA, HIV viral DNA, HIV proviral DNA, or HIV viral protein in a subject. In one embodiment, the method comprises providing to the subject in need thereof an amount of an antibody effective to prevent an increase in HIV viral load, virus replication or an amount of an HIV protein of one or more HIV strains or isolates in the subject. In certain embodiments, the method further comprises measuring an amount of HIV viral RNA, DNA, or proviral DNA or protein at one or more time points, e.g., before and after the subject is administered the antibody or one or more polynucleotides.

An antibody of the present disclosure may be administered to a subject using any route of administration, e.g., systemic, parenterally, locally, in accordance with known methods. Such routes include, but are not limited to, intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. A subject may be administered an antibody of the present invention one or more times; and may be administered before, after, or concurrently with another therapeutic agent as further described below.

In certain embodiments, the antibody or antigen-binding fragment thereof of the present invention is provided to the subject in combination with one or more additional therapeutic agents used to treat HIV infection or a related disease or disorder. In certain embodiments, a method for treating or preventing an HIV infection in a mammal, e.g., a human, having or at risk of having the infection is provided, comprising administering to the human a therapeutically effective amount of an antibody as disclosed herein, or a pharmaceutically acceptable salt thereof, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of an antibody as disclosed herein, or a pharmaceutically acceptable salt thereof, in combination with a therapeutically effective amount of one or more (e.g., one, two, three, one or two, or one to three) additional therapeutic agents.

In some embodiments, two or more antibodies of the present disclosure may be administered to the subject. In some embodiments, the two or more antibodies may have different neutralization capabilities, i.e., they exhibit a different neutralization profiles for different HIV strain or combinations of strains, as compared to each other. In some embodiments, the antibody may be administered with another anti-HIV therapeutic antibody.

In some embodiments, an additional therapeutic agent may be an anti-HIV agent. For example, in some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., PD-1 modulators, PD-L1 modulators, toll like receptors modulators, IL-15 agonists), other HIV antibodies, bispecific antibodies and “antibody-like” therapeutic proteins (e.g., DARTs®, Duobodies®, Bites®, XmAbs®, TandAbs®, Fab derivatives) including those targeting HIV gp120 or gp41, combination drugs for HIV, HIV p17 matrix protein inhibitors, IL-13 antagonists, Peptidyl-prolyl cis-trans isomerase A modulators, Protein disulfide isomerase inhibitors, Complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, Integrin antagonists, Nucleoprotein inhibitors, Splicing factor modulators, COMM domain containing protein 1 modulators, HIV Ribonuclease H inhibitors, Retrocyclin modulators, CDK-9 inhibitors, Dendritic ICAM-3 grabbing nonintegrin 1 inhibitors, HIV GAG protein inhibitors, HIV POL protein inhibitors, Complement Factor H modulators, Ubiquitin ligase inhibitors, Deoxycytidine kinase inhibitors, Cyclin dependent kinase inhibitors Proprotein convertase PC9 stimulators, ATP dependent RNA helicase DDX3X inhibitors, reverse transcriptase priming complex inhibitors, HIV gene therapy, PI3K inhibitors, compounds such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences) and WO 2013/006792 (Pharma Resources), and other drugs for treating HIV, and combinations thereof. In some embodiments, the additional therapeutic is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In some embodiments, the additional therapeutic agent is a latency reversing agent (LRA), e.g., a TLR7 agonist. In other embodiments, the additional therapeutic agent is a latency reversing agent (LRA), e.g., a TLR8 agonist. Examples of TLR agonists include but are not limited to Vesatolimod. Additional examples include but are not limited to the compounds described in U.S. Pat. No. 8,367,670 and the compounds described in U.S. Patent Application Publication No. 2016-0289229. In one embodiment, the antibody of the present invention may be combined with TLR7 agonist such as Vesatolimod. In another embodiment, the antibody of the present invention may be combined with TLR8 agonist. In one embodiment, the additional therapeutic agent is a TLR modulator. TLR modulators may include modulators of TLR1, TLR2, TLR3, TLR4, TLR5, TLR6, TLR7, TLR8, TLR9, TLR 10, TLR11, TLR 12, and TLR13. Examples of TLR3 modulators include rintatolimod, poly-ICLC, RIBOXXON®, Apoxxim, RIBOXXIM®, IPH-33, MCT-465, MCT-475, and ND-1.1. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences). Examples of TLR8 modulators include motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10.

In some embodiments, the additional therapeutic agents comprise one or more antiretroviral therapies (ARTs). In some embodiments, the ART is a combination ART (cART) such as highly active ART (HAART). In some embodiments, the ART comprises one or more of a nucleoside reverse transcriptase inhibitor (NRTI), a non-nucleoside reverse transcriptase inhibitor (NNRTI), a protease inhibitor (PI), an entry inhibitor, or an HIV integrase inhibitor. Examples of NRTIs include but are not limited to: Zidovudine (Retrovir, AZT); Didanosine (Videx, Videx EC, ddl); Stavudine (Zerit, d4T); Lamivudine (Epivir, 3TC); Tenofovir, a nucleotide analog (Viread, TDF); Combivir (combination of zidovudine and lamivudine); Trizivir (combination of zidovudine, lamivudine and abacavir); Emtricitabine (Emtriva, FTC); Truvada (combination of emtricitabine and tenofovir); and Epzicom (combination of abacavir and lamivudine). Examples of NNRTIs include but are not limited to: Nevirapine (Viramune, NVP); Delavirdine (Rescriptor, DLV); Efavirenz (Sustiva or Stocrin, EFV, also part of Atripla); Etravirine (Intelence, ETR); and Rilpivirine (Edurant, RPV, also part of Complera or Epivlera). Examples of Pis include but are not limited to: Saquinavir (Invirase, SQV); Indinavir (Crixivan, IDV); Ritonavir (Norvir, RTV); Nelfinavir (Viracept, NFV); Amprenavir (Agenerase, APV); Lopinavir/ritonavir (Kaletra or Aluvia, LPV/RTV); Atazanavir (Reyataz, ATZ); Fosamprenavir (Lexiva, Telzir, FPV); Tipranavir (Aptivus, TPV); and Darunavir (Prezista, DRV). Examples of entry inhibitors include but are not limited to: Enfuvirtide (Fuzeon, ENF, T-20) and Maraviroc (Selzentry or Celsentri, MVC). Examples of HIV integras inhibitors include but are not limited to: Raltegravir (Isentress, RAL); Elvitegravir (EVG, part of the combination Stribild) and Dolutegravir (Tivicay, DTG).

In some embodiments, an anti-HIV antibody of the present invention is administered with a latency reversing agents (e.g., histone deacetylase inhibitors, proteasome inhibitors, protein kinase C (PKC) activators, bromo and external bromodomain inhibitors, acetaldehyde dehydrogenase inhibitors, and activators of nuclear factor kappa-light chain-enhancer of activated B cells (NF-κB) and the AKT pathway. In some embodiments, the latency reversing agent is a TLR7 agonist. In other embodiments, the latency reversing agent is a TLR8 agonist. Examples of TLR agonists include but are not limited to Vesatolimod. Additional examples include but are not limited to the compounds described in U.S. Pat. No. 8,367,670 and the compounds described in U.S. Patent Application Publication No. 2016-0289229. In one embodiment, the antibody of the present invention may be combined with TLR7 agonist such as Vesatolimod. In another embodiment, the antibody of the present invention may be combined with a TLR8 agonist. In one embodiment, the additional therapeutic agent is a TLR modulator. TLR modulators may include modulators of TLR1, TLR2, TLR3, TLR4, TLR5, TLR6, TLR7, TLR8, TLR9, TLR 10, TLR11, TLR 12, and TLR13. Examples of TLR3 modulators include rintatolimod, poly-ICLC, RIBOXXON®, Apoxxim, RIBOXXIM®, IPH-33, MCT-465, MCT-475, and ND-1.1. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences). Examples of TLR8 modulators include motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10. In some embodiments, the latency reversing agent is a PKC agonist such as bryostatin-1, prostratin, ingenol-3-angelate, ingenol mimic, or DAG mimic. In certain embodiments, the latency reversing agent is an activator of NF-κB such as disulfiram. In certain embodiments, the latency reversing agent is a histone deacetylase inhibitor selected from the group consisting of vorinostat, panobinostat, and romidepsin. In other embodiments, the histone deacetylase inhibitor is selected from 4-phenylbutyrohydroxamic acid, Acetyldinaline, APHA, Apicidin, AR-42, Belinostat, CUDC-101, CUDC-907, Dacinostat, Depudecin, Droxinostat, Entinostat, Givinostat, HC-Toxin, ITF-2357, JNJ-26481585, KD 5170, LAQ-824, LMK 235, M344, MC1568, MGCD-0103, Mocetinostat, NCH 51, Niltubacin, NSC3852, Oxamflatin, Panobinostat, PCI-24781, PCI-34051, Pracinostat, Pyroxamide, Resminostat, RG2833, RGFP966, Rocilinostat, Romidepsin, SBHA, Scriptaid, Suberohydroxamic acid, Tacedinaline, TC-H 106, TCS HDAC6 20b, Tacedinaline, TMP269, Trichostatin A, Tubacin, Tubastatin A, Valproic acid, or Vorinostat. In certain embodiments, the latency reversing agent is a bromodomain inhibitor such as JQ1. In other embodiments, the inhibitor is selected from CPI 203, 1-BET151, 1-BET762, JQ1, MS417, MS436, OTX-015, PFi-1, or RVX-208. In certain embodiments, a combination of latency reversing agents is administered with an anti-HIV antibody of the present invention. The antibody may be administered simultaneously or sequentially, either before or after, with one or more latency reversing agents. In some embodiments, a subject may additionally undergo treatment with another therapeutic agent for HIV infection.

In some embodiments, an antibody of the present disclosure is formulated as a tablet, which may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In some embodiments, such tablets are suitable for once daily dosing.

In some embodiments, an anti-HIV antibody of the present disclosure is administered with an additional therapeutic agent selected from one or more of: (1) Combination drugs selected from the group consisting of ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), dolutegravir+abacavir sulfate+lamivudine, TRIUMEQ® (dolutegravir+abacavir+lamivudine), lamivudine+nevirapine+zidovudine, dolutegravir+rilpivirine, dolutegravir+rilpivirine hydrochloride, atazanavir sulfate+cobicistat, atazanavir+cobicistat, darunavir+cobicistat, efavirenz+lamivudine+tenofovir disoproxil fumarate, tenofovir alafenamide hemifumarate+emtricitabine+cobicistat+elvitegravir, tenofovir alafenamide hemifumarate+emtricitabine, tenofovir alafenamide+emtricitabine, tenofovir alafenamide hemifumarate+emtricitabine+rilpivirine, tenofovir alafenamide+emtricitabine+rilpivirine, Vacc-4x+romidepsin, darunavir+tenofovir alafenamide hemifumarate+emtricitabine+cobicistat, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (Livexa®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), doravirine+lamivudine+tenofovir disoproxil fumarate, doravirine+lamivudine+tenofovir disoproxil, tenofovir+lamivudine and lamivudine+tenofovir disoproxil fumarate; (2) HIV protease inhibitors selected from the group consisting of amprenavir, atazanavir, fos amprenavir, fosamprenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100) and TMC-310911; (3) HIV non-nucleoside or non-nucleotide inhibitors of reverse transcriptase selected from the group consisting of delavirdine, delavirdine mesylate, nevirapine, etravirine, dapivirine, doravirine, rilpivirine, efavirenz, KM-023, VM-1500, lentinan and AIC-292; (4) HIV nucleoside or nucleotide inhibitors of reverse transcriptase selected from the group consisting of VIDEX® and VIDEX® EC (didanosine, ddl), zidovudine, emtricitabine, didanosine, stavudine, zalcitabine, lamivudine, censavudine, abacavir, abacavir sulfate, elvucitabine, alovudine, phosphazid, fozivudine tidoxil, apricitabine, KP-1461, fosalvudine tidoxil, tenofovir, tenofovir disoproxil, tenofovir disoproxil fumarate, tenofovir disoproxil hemifumarate, tenofovir alafenamide, tenofovir alafenamide hemifumarate, tenofovir alafenamide fumarate, adefovir, adefovir dipivoxil, and festinavir; (5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, elvitegravir, dolutegravir and cabotegravir; (6) HIV non-catalytic site, or allosteric, integrase inhibitors (NCIND selected from the group consisting of CX-05168, CX-05045 and CX-14442; (7) HIV gp41 inhibitors selected from the group consisting of enfuvirtide, sifuvirtide and albuvirtide; (8) HIV entry inhibitors selected from the group consisting of cenicriviroc; (9) HIV gp120 inhibitors selected from the group consisting of Radha-108 (Receptol) and BMS-663068; (10) CCR5 inhibitors selected from the group consisting of aplaviroc, vicriviroc, maraviroc, cenicriviroc, PRO-140, Adaptavir (RAP-101), nifeviroc (TD-0232), TD-0680, and vMIP (Haimipu); (11) CD4 attachment inhibitors, e.g., Fostemsavir (BMS-663068); (12) inhibitors of post-binding events required for entry selected from the group consisting of ibalizumab and CXCR4 inhibitors such as plerixafor, ALT-1188, vMIP and Haimipu; (13) Pharmacokinetic enhancers selected from the group consisting of cobicistat and ritonavir; (14) Immune-based therapies selected from the group consisting of derma Vir, interleukin-7, plaquenil (hydroxychloroquine), proleukin (aldesleukin, IL-2), interferon alfa, interferon alfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-12, polymer polyethyleneimine (PEI), Gepon, VGV-1, MOR-22, BMS-936559, toll-like receptors modulators (TLR1, TLR2, TLR3, TLR4, TLR5, TLR6, TLR7, TLR8, TLR9, TLR10, TLR11, TLR12 and TLR13), rintatolimod and IR-103; (15) HIV vaccines selected from the group consisting of peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), monomeric gp120 HIV-1 subtype C vaccine (Novartis), Remune, ITV-1, Contre Vir, Ad5-ENVA-48, DCVax-001 (CDX-2401), PEP-6409, Vacc-4x, Vacc-05, VAC-3S, multiclade DNA recombinant adenovirus-5 (rAd5), Pennvax-G, VRC-HIV MAB060-00-AB, AVX-101, AVX-201, HIV-LAMP-vax, Ad35, Ad35-GRIN, NAcGM3/VSSP ISA-51, poly-ICLC adjuvanted vaccines, Tatlmmune, GTU-multiHIV (FIT-06), AGS-004, gp140[delta]V2.TV1+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT 123, rAAVl-PG9DP, GOVX-B 11, GOVX-B21, ThV-01, TUTI-16, VGX-3300, TVI-HIV-1, Ad-4 (Ad4-env Clade C+Ad4-mGag), EN41-UGR7C, EN41-FPA2, PreVaxTat, TL-01, SAV-001, AE-H, MYM-V101, CombiHIVvac, ADVAX, MYM-V201, MVA-CMDR, MVATG-17401, ETV-01, CDX-1401, rcAd26.MOS 1.HIV-Env and DNA-Ad5 gag/pol/nef/nev (HVTN505); (16) HIV antibodies, bispecific antibodies and “antibody-like” therapeutic proteins (such as DARTs®, Duobodies®, Bites®, XmAbs®, TandAbs®, Fab derivatives) including BMS-936559, TMB-360 and those targeting HIV gp120 or gp41 selected from the group consisting of bavituximab, UB-421, C2F5, C2G12, C4E10, C2F5+C2G12+C4E10, 3-BNC-117, PGT145, PGT121, MDX010 (ipilimumab), VRCOl, A32, 7B2, 10E8, VRC-07-523 and VRC07; (17) latency reversing agents selected from the group consisting of Histone deacetylase inhibitors such as Romidepsin, vorinostat, panobinostat; Proteasome inhibitors such as Velcade; protein kinase C (PKC) activators such as Indolactam, Prostratin, Ingenol B and DAG-lactones, Ionomycin, GSK-343, PMA, SAHA, BRD4 inhibitors, IL-15, JQ1, disulfram, and amphotericin B; (18) HIV nucleocapsid p7 (NCp7) inhibitors selected from the group consisting of azodicarbonamide; (19) HIV maturation inhibitors selected from the group consisting of BMS-955176 and GSK-2838232; (20) PI3K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401; (21) the compounds disclosed in WO 2004/096286 (Gilead Sciences), WO 2006/110157 (Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences); and (22) other drugs for treating HIV selected from the group consisting of BanLec, MK-8507, AG-1105, TR-452, MK-8591, REP 9, CYT-107, alisporivir, NOV-205, IND-02, metenkefalin, PGN-007, Acemannan, Gamimune, Prolastin, 1,5-dicaffeoylquinic acid, BIT-225, RPI-MN, VSSP, Hlviral, IMO-3100, SB-728-T, RPI-MN, VIR-576, HGTV-43, MK-1376, rHIV7-shl-TAR-CCRSRZ, MazF gene therapy, BlockAide, ABX-464, SCY-635, naltrexone, AAV-eCD4-Ig gene therapy, TEV-90110, TEV-90112, deferiprone, and PA-1050040 (PA-040).

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In some embodiments, an antibody as disclosed herein is combined with one or more additional therapeutic agents in a unitary dosage form for simultaneous administration to a patient, for example as a solid dosage form for oral administration.

“Co-administration” of an antibody as disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient.

Co-administration includes administration of unit dosages of the antibody disclosed herein before or after administration of unit dosages of one or more additional therapeutic agents, for example, administration of the antibody within seconds, minutes, or hours of the administration of one or more additional therapeutic agents. For example, in some embodiments, a unit dose of an antibody disclosed herein is administered first, followed within seconds or minutes by administration of a unit dose of one or more additional therapeutic agents. Alternatively, in other embodiments, a unit dose of one or more additional therapeutic agents is administered first, followed by administration of a unit dose of an antibody within seconds or minutes. In some embodiments, a unit dose of an antibody disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another antigen associated with the HIV infectious agent.

As described herein, the antibody may also be administered by gene therapy by administration of a nucleic acid comprising one or more polynucleotides encoding the antibody. In certain embodiments, the polynucleotide encodes an scFv. In particular embodiments, the polynucleotide comprises DNA, cDNA or RNA. In certain embodiments, the polynucleotide is present in a vector, e.g., a viral vector

The following examples are offered for illustrative purposes, and are not intended to limit the invention. Those of skill in the art will readily recognize a variety of non-critical parameters that can be changed or modified to yield essentially the same results.

### EXAMPLES

### Example 1. Generation of Anti-HIV Antibody Variants

Patient-derived broadly neutralizing antibodies against HIV were identified from a donor, NVS49. These antibodies included the lineage L1, which include antibodies L1A1, L1A2, and L1A4. The example describes antibody variants of L1A2.

Variants were designed to improve activity and to remove liabilities that can cause undesirable antibody properties, resulting in delays in development, increased development costs, failure in development, or increased product costs. Desired antibody properties comprise: (1) suitable for a standard platform (expression, purification, formulation); (2) high yield; (3) low heterogeneity (glycosylation, chemical modification, etc.); (4) consistent manufacturability (batch-to-batch, and small-to-large scale); (5) high stability (years in liquid formulation), e.g., minimal chemical degradation, fragmentation, and aggregation; and (6) long PK (in vivo half life), e.g., no off-target binding, no impairment of FcRn recycling, and stable. Illustrative motifs considered during design of variants included the following. The “Risk” category is assigned in this table based on the likelihood of having the impact shown in Column 2.

TABLE 4
Description of potential (risk) of development liabilities

| Property | Potential Impact | Seq. motif | Risk |
|---|---|---|---|
| Free cysteine | Yield, heterogeneity, | Odd # C | High |
| | stability, activity | | |
| N-linked | Yield, heterogeneity, | N(~P)(S, T) | High |
| glycosylation | activity | | |
| (“Nglyco”) | | | |
| Abnormal net | Platform fit, PK | [Sharma 2014] | High |
| charge | | | |
| Patches of | Stability, PK | [Sharma 2014] | High |
| hydrophobicity | | | |
| Patches of same | Stability, PK | N/A (based on | Medium |
| charge | | structure) | |
| Proteolysis | Stability, PK | (K, R)(K, R) | Medium |
| Proteolysis | Stability, PK | DP | Medium |
| Asparagine | Heterogeneity, | NG; | Medium; |
| deamidation | stability, activity | N(A, N, S, T)* | Low |
| Aspartate | Heterogeneity, | DG; | Medium; |
| isomerization | stability, activity | D(A, D, S, T)* | Low |
| Lysine glycation | Heterogeneity, | K | Low |
| | stability, activity | | |
| Methionine | Heterogeneity, | M | Low |
| oxidation | stability, activity | | |
| Tryptophan | Heterogeneity, | W | Low |
| oxidation | stability, activity | | |

*N and D are also potentially low risk for other successor residues, e.g., D, N, H, or P

Another goal in generating variants is to reduce the risk of clinical immunogenicity and the generation of anti-drug antibodies (ADAs) against the therapeutic antibody. To minimize risk of immunogenicity, the lead sequence can be engineered to be as similar to the intended patient population's native (“self”) sequences as possible.

One approach employed to design variants that are as much like self as possible was to identify the closest germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all of the variable heavy (VH) and variable light (VL) regions except for part of H-CDR3. Germline gene IGHD codes for part of the H-CDR3 region but typically exhibits too much variation in how it is recombined with IGHV and IGHJ (eg, forward or reverse orientation, any of three translation frames, and 5′ and 3′ modifications and non-templated additions) to present a “self” sequence template from a population perspective.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for the prediction of HLA binding, and use in vitro assessment to further identify potential epitopes.

Antibody variants are additionally designed to enhance the efficacy of the antibody. Design parameters in this category focused on CDRs. Residues to mutate in the naturally occurring antibody sequences were identified based on sequences of other antibodies in the lineage.

### Approaches to Mutation Design

Development liabilities can be removed or reduced by one or more mutations. Mutations are designed to preserve antibody structure and function while removing or reducing the liability. Mutations to chemically similar residues are a preferred approach, e.g., maintaining size, shape, charge, and/or polarity. Illustrative mutations are described in Table 5.

TABLE 5
Potential development liabilities and
illustrative mutations to reduce risk

| | Seq. motif | | Illustrative |
|---|---|---|---|
| Property | (CDR) | Risk | mutations |
| Free cysteine | Odd # C | High | C→(A, S) |
| N-linked glycosylation | N(~P)(S, T) | High | N→(Q, D, S, A); |
| (“Nglyco”) | | | (S, T)→(A, N) |
| Proteolytic cleavage | (K, R)(K, R) | Medium | K, R→(Q, S, A) |
| Proteolytic cleavage | DP | Medium | D→(E, S, A) |
| Asparagine | NG; | Medium; | N→(Q, S, A); |
| deamidation | N(A, N, S, T)* | Low | G→(A, S) |
| Aspartate | DG; | Medium; | D→(E, S, A); |
| isomerization | D(A, D, S, T)* | Low | G→(A, S) |
| Lysine glycation | K | Low | K→(R, Q, S, A) |
| Methionine oxidation | M | Low | M→(Q, L, S, A) |
| Tryptophan oxidation | W | Low | W→(Y, F) |

L1A2 variants were also designed taking into consideration the sequences of siblings L1A1 and L1A4.

Protein structure can be used to assess the risk of mutating a position or to design a mutation predicted to preserve antibody structure and function. Most preferably is a high resolution crystal structure of the antibody, preferably in complex with its target; alternatively, a homology model can be built to predict the structure of the antibody.

### Assessment of NVS49-L1 Lineage

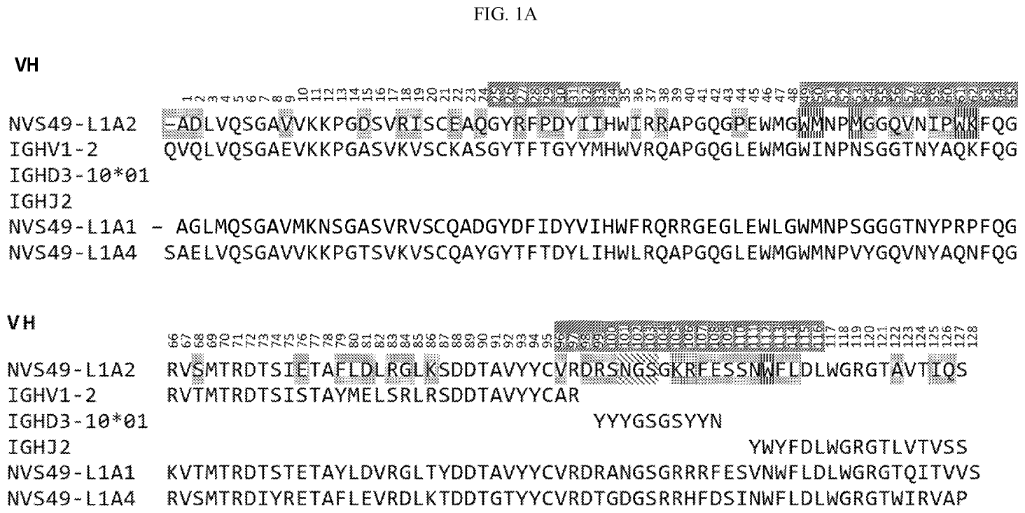

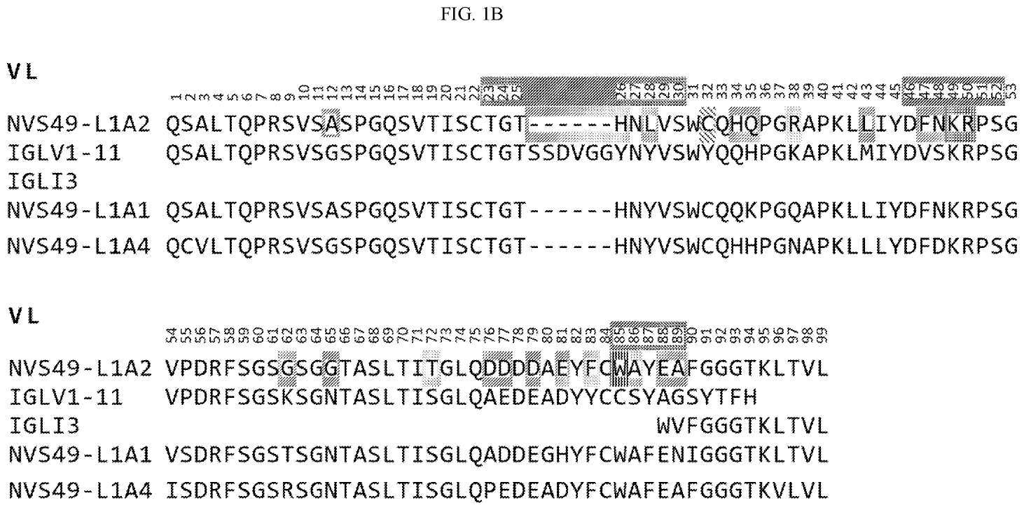

The sequence of L1A2 was aligned to L1A1, L1A4, and closest germline sequences (HV1-2, HD3-10*01, HJ2, LV2-11, and LJ3) using L1A2 as reference (FIGS. 1A and 1B). CDRs, germline deviations, and potential liabilities in L1A2 were identified (shaded positions in FIGS. 1A and 1B). Non-canonical cysteines and N-glycosylation sites were identified across the full V_{H} and V_{L}, whereas the other potential liability motifs were identified only within the CDRs.

Potential PK risk was also estimated based Sharma et al., Proc. Natl. Acad. Sci. USA 111:18601-18606, 2014. High hydrophobicity index (HI) was found to correlate with faster clearance, where HI<5 is preferred to reduce risk, and HI<4 is most preferred to reduce risk. However, some antibodies with HI>4, or HI>5, will not exhibit fast clearance, and be false positives. Secondly, too high or too low Fv charge as calculated at pH 5.5 was found to correlate with faster clearance, where charge between (−2, +8) is preferred to reduce risk, and charge between (0, +6.2) is most preferred to reduce risk.

TABLE 6
Summary of potential liabilities in L1A2

| | L1A2 |
|---|---|
| Odd # C | 1 |
| N(~P)(S, T) | 1 |
| Fv charge 5.5 | 6.00 |
| HI | 5.44 |
| (K, R)(K, R) | 2 |
| DP | 0 |
| NG | 1 |
| N(A, N, S, T) | 0 |
| DG | 0 |
| D(A, D, S, T) | 0 |
| K | 3 |
| M | 2 |
| W | 4 |
| FW non-germ | 37 |
| VH | 23 |
| VL | 14 |

### Design of Variants to Germline L1A2

The 37 framework germline deviations in L1A2 were analyzed for their potential to be mutated, individually or in combination, to germline sequence, without negatively impacting gp120 binding activity. For each of the 37 candidate mutations from L1A2 sequence to germline sequence, the risk of making the mutation was assessed based on: (1) the change in charge, if any, since change in charge is intrinsically risky, and a change to more positive charge has particular risk given the already net positive charge of L1A2 Fv; histidine is approximated as +0.5 charge because its side chain pKa is near physiological pH; (2) conservation of the native L1A2 residue in the lineage versus the presence of the germline residue or other mutations at that position in the lineage, particularly L1A1 or L1A4; (3) the structural location of the position with respect to gp120; (4) the backbone phi-psi conformation of the position and the typical allowed regions of phi-psi space for certain amino acids, particularly if mutation to or from glycine or proline is under consideration; (5) any interactions likely being made between the native side chain and other antibody residues or parts of gp120; and (6) the potential for the germline side chain to fit in the antibody and maintain or enhance interactions with antibody or gp120.

TABLE 7
Assessment of germline mutations in the framework of L1A2

| | | Germline | | |
|---|---|---|---|---|
| | | in an L1A1 | | |
| Germlining | Change in | or L1A4 | | |
| mutation | charge | sibling? | Where | Description |
| H0XQ | 0.0 | No | far from | |
| | | | gp120 | |
| H1AV | 0.0 | No | far from | |
| | | | gp120 | |
| H2DQ | +1.0 | No | far from | coupled with H24QS |
| | | | gp120 | (D<>Q Hbond), and |
| | | | | H22EK |
| H9VE | −1.0 | No | far from | ^{~}near H122AL |
| | | | gp120 | |
| H15DA | +1.0 | Yes | | close to H11K & H12K; |
| | | | | often coupled to |
| | | | | H12K −> N/T |
| H18RK | 0.0 | Yes | | coupled salt bridge to |
| | | | | H81D |
| H19IV | 0.0 | Yes | | |
| H22EK | +2.0 | No | far from | weakly coupled with |
| | | | gp120 | H2DQ & H24QS; risk |
| | | | | from +2 |
| H24QS | 0.0 | No | far from | coupled with H2DQ |
| | | | gp120 | |
| H36IV | 0.0 | Yes | base of CDRs | maybe coupled to H44P |
| | | | | etc |
| H38RQ | −1.0 | Yes | | coupled salt bridge with |
| | | | | L81E; near H44P, L34H |
| H44PL | 0.0 | Yes | | part of L/H region |
| H68ST | 0.0 | Yes | | Hbond from H83R |
| H76ES | +1.0 | No | somewhat | near H22/24 |
| | | | close | |
| H79FY | 0.0 | Yes | | also reduces surface |
| | | | | hydrophobic patch |
| H80LM | 0.0 | No | | |
| H81DE | 0.0 | Yes | | coupled salt bridge to |
| | | | | H18R |
| H83RS | −1.0 | Yes | | charge coupled to |
| | | | | H84GR. Hbond to H68S, |
| | | | | H66Oxy |
| H84GR | +1.0 | No | | charge coupled to H83RS |
| H86KR | 0.0 | Yes | | could salt bridge with |
| | | | | H88D |
| H122AL | 0.0 | No | far from | ^{~}near H9VE |
| | | | gp120 | |
| H125IV | 0.0 | Yes | far from | |
| | | | gp120 | |
| H126QS | 0.0 | No | far from | |
| | | | gp120 | |
| L12AG | 0.0 | yes; | | |
| L32CY | 0.0 | No | | maybe part of L/H region |
| L34HQ | −0.5 | Yes | | part of L/H region |
| L35QH | +0.5 | Yes | | Hbond with L33Q |
| L38RK | 0.0 | No | | near L41K, H119R |
| L43LM | 0.0 | No | base of CDRs | could have significant |
| | | | | influence |
| L62GK | +1.0 | No | near L1 & L2 | backbone Gly-specific |
| L65GN | 0.0 | Yes | near L1 & L2 | backbone Gly-specific |
| L72TS | 0.0 | Yes | | |
| L76DA | +1.0 | Yes | | |
| L77DE | 0.0 | Yes | | fair # of changes in region |
| L79DE | 0.0 | Yes | | Hbond to L98NH |
| L81ED | 0.0 | Yes | | coupled salt bridge with |
| | | | | H38R |
| L83FY | 0.0 | No | | near H38R, L81E |

Positions in the V_{H} and V_{L} regions of L1 A2 that can be varied are shown in Table 13.

### Variants Comprising Multiple Mutations

Combination of variants were designed based on combining the lowest predicted-risk mutations, then adding additional relatively riskier mutations into more highly mutated variants (Table 8). The proposed combinations are not limiting, in that many of the germline mutations can be made without significant impact to the function; and various combination of mutations can also be employed in a variant.

TABLE 8
Combinations of germline mutations for L1A2

| | Germ6 | Germ12 | Germ18 | Germ17 | Germ23 | Germ29 | Germ33 |
|---|---|---|---|---|---|---|---|
| | Value of 1 means the mutation is included in variant | | | | | | |
| | Low risk | All | All low & | All low & | All low & | All low | All low & |
| | & in L1A1 | low | some VH | some VL | some VL, some | & med | med, some |
| | or L1A4 | risk | med risk | med risk | VH med risk | risk | high risk |
| H0XQ | 0 | 1 | 1 | 1 | 1 | 1 | 1 |
| H1AV | 0 | 1 | 1 | 1 | 1 | 1 | 1 |
| H2DQ | 0 | 0 | 1 | 0 | 1 | 1 | 1 |
| H9VE | 0 | 0 | 1 | 0 | 1 | 1 | 1 |
| H15DA | 0 | 0 | 1 | 0 | 1 | 1 | 1 |
| H18RK | 0 | 0 | 1 | 0 | 1 | 1 | 1 |
| H19IV | 1 | 1 | 1 | 1 | 1 | 1 | 1 |
| H22EK | 0 | 0 | 0 | 0 | 0 | 0 | 0 |
| H24QS | 0 | 0 | 1 | 0 | 1 | 1 | 1 |
| H36IV | 0 | 0 | 0 | 0 | 0 | 1 | 1 |
| H38RQ | 0 | 0 | 0 | 0 | 0 | 0 | 1 |
| H44PL | 0 | 0 | 0 | 0 | 0 | 0 | 1 |
| H68ST | 0 | 0 | 0 | 0 | 0 | 1 | 1 |
| H76ES | 0 | 0 | 0 | 0 | 0 | 1 | 1 |
| H79FY | 1 | 1 | 1 | 1 | 1 | 1 | 1 |
| H80LM | 0 | 1 | 1 | 1 | 1 | 1 | 1 |
| H81DE | 0 | 0 | 1 | 0 | 1 | 1 | 1 |
| H83RS | 0 | 0 | 0 | 0 | 0 | 1 | 1 |
| H84GR | 0 | 0 | 0 | 0 | 0 | 1 | 1 |
| H86KR | 0 | 1 | 1 | 1 | 1 | 1 | 1 |
| H122AL | 0 | 1 | 1 | 1 | 1 | 1 | 1 |
| H125IV | 1 | 1 | 1 | 1 | 1 | 1 | 1 |
| H126QS | 0 | 1 | 1 | 1 | 1 | 1 | 1 |
| L12AG | 1 | 1 | 1 | 1 | 1 | 1 | 1 |
| L32CY | 0 | 0 | 0 | 0 | 0 | 0 | 0 |
| L34HQ | 0 | 0 | 0 | 0 | 0 | 1 | 1 |
| L35QH | 0 | 0 | 0 | 1 | 1 | 1 | 1 |
| L38RK | 0 | 0 | 0 | 1 | 1 | 1 | 1 |
| L43LM | 0 | 0 | 0 | 1 | 1 | 1 | 1 |
| L62GK | 0 | 0 | 0 | 0 | 0 | 0 | 0 |
| L65GN | 0 | 0 | 0 | 0 | 0 | 0 | 0 |
| L72TS | 1 | 1 | 1 | 1 | 1 | 1 | 1 |
| L76DA | 0 | 0 | 0 | 1 | 1 | 1 | 1 |
| L77DE | 1 | 1 | 1 | 1 | 1 | 1 | 1 |
| L79DE | 0 | 0 | 0 | 1 | 1 | 1 | 1 |
| L81ED | 0 | 0 | 0 | 0 | 0 | 0 | 1 |
| L83FY | 0 | 0 | 0 | 0 | 0 | 0 | 1 |
| Total # | 6 | 12 | 18 | 17 | 23 | 29 | 33 |
| Mutations | | | | | | | |
| Change in | 0.0 | 0.0 | +1.0 | +1.5 | +2.5 | +3.0 | +2.0 |
| net charge: | | | | | | | |

Design of Variants to Remove Liabilities from L1A2

The 14 sequence-based liabilities in L1 A2 were analyzed for their potential to be mutated to reduce or remove the risk of liability while not negatively impacting gp120 binding activity. In addition, four residues were identified which either contribute to the hydrophobicity index (HI) value of 5.44 (>5 indicates increased risk of fast clearance) or contribute to other surface hydrophobicity.

For each of the 18 residues, mutations were designed to address the liability while aiming to preserve function. Similar to the germlining design, risk was assessed based on change in charge, shape, polarity, backbone conformation preference, and maintenance or enhancement of side chain interactions (Table 9).

TABLE 9
Design of variants to remove liabilities from L1A2

| Liability | | | | |
|---|---|---|---|---|
| position(s) | Location | Liability type | Mutation(s) | Description |
| H49W | H-CDR2 | Trp oxidation | H49WY | Trp makes direct contact and |
| | | | | Hbond to gp120 |
| H50M | H-CDR2 | Met | H50MI | Ile in germline; buried and |
| | | oxidation | | second shell |
| H53M | H-CDR2 | Met | H53MS | solvent exposed and makes 2 |
| | | oxidation | | rough gp120 contacts. S in |
| | | | | L1A1 |
| | | | H53MV | V in L1A4 |
| H61W | H-CDR2 | Trp oxidation | H61WQ | near but not quite contacting |
| | | & surface | | gp120. Q in L1A4; R in L1A1. |
| | | hydrophobic | | Also germlines |
| H62K | H-CDR2 | Lys glycation | H62KN | salt bridge to H45E and near |
| | | | | H37R. Periphery of gp120. P |
| | | | | in L1A12; N in L1A4. |
| H75I | H-FW3 | surface | H75IT | Ile is germline and the |
| | | hydrophobic | | hydrophobic patch would |
| | | | | already be reduced by the |
| | | | | germlining mutation H79FY |
| H101N- | H-CDR3 | N- | H101ND | Asp in L1A4 |
| H102G- | | glycosylation | | |
| H103S | | | | |
| | | | H27RT + | Brings 6 more of L1A4 |
| | | | H100SG + | |
| | | | H101ND + | |
| | | | H104GR + | |
| | | | H105KR + | |
| | | | H106RH + | |
| | | | H108ED | |
| | | | H103SW | Significant change |
| | | | H103SA | Ser is not making contacts, so |
| | | | | Ala is a candidate |
| | | | H101NS | |
| | | | H101NA | |
| H101N- | H-CDR3 | Asn | H101NS or | H102G backbone phi-psi is |
| H102G | | deamidation | H101NA | approx (+120, −170) which is |
| | | | | disallowed for general side |
| | | | | chains, so preferable to not |
| | | | | mutate the Gly |
| H105K- | H-CDR3 | Dibasic | H105KQ | Arg is pointed at gp120 but |
| H106R | | proteolysis | | Lys is pointing away. |
| | | | H105KS | |
| | | | H105KA | |
| H105K | H-CDR3 | Lys glycation | see H105Kx | |
| | | | above | |
| H107F | H-CDR3 | HI = 5.44 (>4) | H107FY | reduce HI from 5.44 to 5.38 |
| | | | | (only), but adds solvent |
| | | | | exposed hydroxyl, which can |
| | | | | Hbond to L45Y. Still F in L1A1 |
| | | | | and L1A4 |
| H112W | H-CDR3 | Trp oxidation | H112WY | Intramolecular packing and |
| | | | | contacts and Hbond to gp120; |
| | | | | Trp in L1A1 and L1A4 |
| L28L | L-CDR1 | HI = 5.44 (>4) | L28LY | reduce HI from 5.44 to 4.90 |
| | | | | (toward cutoff of 4). Also |
| | | | | germlines. Tyr in L1A1 & |
| | | | | L1A4, which also have |
| | | | | H110SV or H110SI, but may |
| | | | | not be structurally coupled. |
| | | | | Near H3 and gp120 Nglyco |
| L32C | L-FW2 | Free cysteine | L32CA | Cys is buried so risk is |
| | | | | medium, not high, and |
| | | | | preferable substitution is |
| | | | | hydrophobic and small. Ala is |
| | | | | 1 heavy atom smaller than |
| | | | | Cys so may affect |
| | | | | conformation and L/H |
| | | | | orientation |
| | | | L32CV | Val is 1 heavy atom larger |
| | | | | than Cys and beta branched |
| | | | | so may have negative |
| | | | | influence. However, |
| | | | | modeling indicates that size |
| | | | | of Val can fit, and backbone |
| | | | | phi/psi of (−149, +134) is in |
| | | | | beta strand which a beta- |
| | | | | branched Val can fit into |
| | | | L32CL | Leu is 2 heavy atoms larger |
| | | | | than Cys. However, modeling |
| | | | | suggests that Leu can fit. |
| | | | L32CI | Ile is also 2 heavy atoms |
| | | | | larger than Cys, but also beta |
| | | | | branched, Ile may fit and if |
| | | | | so, likely that either Val or |
| | | | | Leu will also fit |
| L49K-L50R | L-CDR2 | Dibasic | L49KQ | KR in germline and L1A1 and |
| | | proteolysis | | L1A4. K out in solvent but R |
| | | | | interacting with L58oxy and |
| | | | | near L56D |
| | | | L49KS | |
| | | | L49KA | |
| L49K | L-CDR2 | Lys glycation | see L49Kx | |
| | | | above | |
| L85W | L-CDR3 | Trp oxidation | L85WF | totally buried and interacting |
| | | | | with self. W in L1A1 and L1A4 |
| L89A | L-CDR3 | HI = 5.44 (>4) | L89AN | reduce HI from 5.44 to 3.43 |
| | | | | (to under cutoff of 4). Asn in |
| | | | | L1A1 and room in structure |
| | | | | for it, with potential intra- or |
| | | | | inter-Hbonds |

Table 10 lists illustrative variants and modifications introduced into the variants to remove liabilities.

TABLE 10
L1A2 mutations to address liabilities and combinations thereof

| | | | # | |
|---|---|---|---|---|
| Name | Purpose | Description | Mut | ΔCharge |
| Germ6 | Germline | Low risk based on analysis | 6 | 0.0 |
| | | in Table 7 & in L1A1 or | | |
| | | L1A4 | | |
| Germ12 | Germline | All low risk based on | 12 | 0.0 |
| | | analysis in Table 7 | | |
| Germ18 | Germline | All low & some H med risk | 18 | +1.0 |
| | | based on analysis in Table | | |
| | | 7 | | |
| Germ17 | Germline | All low & some L med risk | 17 | +1.5 |
| | | based on analysis in Table | | |
| | | 6 | | |
| Germ23 | Germline | All low & some L, H med | 23 | +2.5 |
| | | risk based on analysis in | | |
| | | Table 7 | | |
| NglycoSA | Remove Nglyco | H103SA | 1 | 0.0 |
| NglycoND | Remove Nglyco | H101ND | 1 | −1.0 |
| NglycoNDplus6 | Remove Nglyco | H101ND + 6 more of L1A4 | 7 | −1.5 |
| CysCA | Remove Cys | L32CA | 1 | 0.0 |
| CysCV | Remove Cys | L32CV | 1 | 0.0 |
| Hydro2 | Reduce | L89AN + H61WQ | 2 | 0.0 |
| | hydrophobic | | | |
| | surface | | | |
| Germ12_NglycoSA_CysCA | Germline + Remove | All low risk based on | 14 | 0.0 |
| | Nglyco + Remove | analysis in Table 7 + | | |
| | Cys | H103SA + L32CA | | |

Illustrative L1A2 variant antibody sequences comprising modifications and combinations of modifications to remove high-liability residues and/or improve activity are described in Tables 11 and 12. The proposed combinations are not limiting, e.g., many of the germline mutations can be made without significant impact to the function; and various combination of mutations can also be employed in a variant.

Table 11 provides illustrative sequences of L1A2 variants that have modifications to address liabilities or improve activity, including variants that comprise combinations of modifications. Antibodies listed in Table 11 were evaluated for binding and/or neutralization activity.

TABLE 11

| Name | VH amino acid sequence | VL amino acid sequence |
|---|---|---|
| Germ6 | ADLVQSGAVVKKPGDSVRVSCEAQG | QSALTQPRSVSGSPGQSVTIS |
| | YRFPDYIIHWIRRAPGQGPEWMGWM | CTGTHNLVSWCQHQPGRAP |
| | NPMGGQVNIPWKFQGRVSMTRDTSIE | KLLIYDFNKRPSGVPDRFSGS |
| | TAYLDLRGLKSDDTAVYYCVRDRSN | GSGGTASLTISGLQDEDDAE |
| | GSGKRFESSNWFLDLWGRGTAVTVQ | YFCWAYEAFGGGTKLTVL |
| | S (SEQ ID NO: 3) | (SEQ ID NO: 4) |
| Germ12 | QVDLVQSGAVVKKPGDSVRVSCEAQ | QSALTQPRSVSGSPGQSVTIS |
| | GYRFPDYIIHWIRRAPGQGPEWMGW | CTGTHNLVSWCQHQPGRAP |
| | MNPMGGQVNIPWKFQGRVSMTRDTS | KLLIYDFNKRPSGVPDRFSGS |
| | IETAYMDLRGLRSDDTAVYYCVRDR | GSGGTASLTISGLQDEDDAE |
| | SNGSGKRFESSNWFLDLWGRGTLVT | YFCWAYEAFGGGTKLTVL |
| | VSS (SEQ ID NO: 5) | (SEQ ID NO: 6) |
| Germ18 | QVQLVQSGAEVKKPGASVKVSCEAS | QSALTQPRSVSGSPGQSVTIS |
| | GYRFPDYIIHWIRRAPGQGPEWMGW | CTGTHNLVSWCQHQPGRAP |
| | MNPMGGQVNIPWKFQGRVSMTRDTS | KLLIYDFNKRPSGVPDRFSGS |
| | IETAYMELRGLRSDDTAVYYCVRDRS | GSGGTASLTISGLQDEDDAE |
| | NGSGKRFESSNWFLDLWGRGTLVTV | YFCWAYEAFGGGTKLTVL |
| | SS (SEQ ID NO: 7) | (SEQ ID NO: 8) |
| Germ17 | QVDLVQSGAVVKKPGDSVRVSCEAQ | QSALTQPRSVSGSPGQSVTIS |
| | GYRFPDYIIHWIRRAPGQGPEWMGW | CTGTHNLVSWCQHHPGKAP |
| | MNPMGGQVNIPWKFQGRVSMTRDTS | KLMIYDFNKRPSGVPDRFSG |
| | IETAYMDLRGLRSDDTAVYYCVRDR | SGSGGTASLTISGLQAEDEAE |
| | SNGSGKRFESSNWFLDLWGRGTLVT | YFCWAYEAFGGGTKLTVL |
| | VSS (SEQ ID NO: 9) | (SEQ ID NO: 10) |
| Germ23 | QVQLVQSGAEVKKPGASVKVSCEAS | QSALTQPRSVSGSPGQSVTIS |
| | GYRFPDYIIHWIRRAPGQGPEWMGW | CTGTHNLVSWCQHHPGKAP |
| | MNPMGGQVNIPWKFQGRVSMTRDTS | KLMIYDFNKRPSGVPDRFSG |
| | IETAYMELRGLRSDDTAVYYCVRDRS | SGSGGTASLTISGLQAEDEAE |
| | NGSGKRFESSNWFLDLWGRGTLVTV | YFCWAYEAFGGGTKLTVL |
| | SS (SEQ ID NO: 11) | (SEQ ID NO: 12) |
| NglycoSA | ADLVQSGAVVKKPGDSVRISCEAQG | QSALTQPRSVSASPGQSVTIS |
| | YRFPDYIIHWIRRAPGQGPEWMGWM | CTGTHNLVSWCQHQPGRAP |
| | NPMGGQVNIPWKFQGRVSMTRDTSIE | KLLIYDFNKRPSGVPDRFSGS |
| | TAFLDLRGLKSDDTAVYYCVRDRSN | GSGGTASLTITGLQDDDDAE |
| | GAGKRFESSNWFLDLWGRGTAVTIQS | YFCWAYEAFGGGTKLTVL |
| | (SEQ ID NO: 13) | (SEQ ID NO: 14) |
| NglycoND | ADLVQSGAVVKKPGDSVRISCEAQG | QSALTQPRSVSASPGQSVTIS |
| | YRFPDYIIHWIRRAPGQGPEWMGWM | CTGTHNLVSWCQHQPGRAP |
| | NPMGGQVNIPWKFQGRVSMTRDTSIE | KLLIYDFNKRPSGVPDRFSGS |
| | TAFLDLRGLKSDDTAVYYCVRDRSD | GSGGTASLTITGLQDDDDAE |
| | GSGKRFESSNWFLDLWGRGTAVTIQS | YFCWAYEAFGGGTKLTVL |
| | (SEQ ID NO: 15) | (SEQ ID NO: 16) |
| NglycoNDplus6 | ADLVQSGAVVKKPGDSVRISCEAQG | QSALTQPRSVSASPGQSVTIS |
| | YTFPDYIIHWIRRAPGQGPEWMGWM | CTGTHNLVSWCQHQPGRAP |
| | NPMGGQVNIPWKFQGRVSMTRDTSIE | KLLIYDFNKRPSGVPDRFSGS |
| | TAFLDLRGLKSDDTAVYYCVRDRGD | GSGGTASLTITGLQDDDDAE |
| | GSRRHFDSSNWFLDLWGRGTAVTIQS | YFCWAYEAFGGGTKLTVL |
| | (SEQ ID NO: 17) | (SEQ ID NO: 18) |
| CysCA | ADLVQSGAVVKKPGDSVRISCEAQG | QSALTQPRSVSASPGQSVTIS |
| | YRFPDYIIHWIRRAPGQGPEWMGWM | CTGTHNLVSWAQHQPGRAP |
| | NPMGGQVNIPWKFQGRVSMTRDTSIE | KLLIYDFNKRPSGVPDRFSGS |
| | TAFLDLRGLKSDDTAVYYCVRDRSN | GSGGTASLTITGLQDDDDAE |
| | GSGKRFESSNWFLDLWGRGTAVTIQS | YFCWAYEAFGGGTKLTVL |
| | (SEQ ID NO: 19) | (SEQ ID NO: 20) |
| CysCV | ADLVQSGAVVKKPGDSVRISCEAQG | QSALTQPRSVSASPGQSVTIS |
| | YRFPDYIIHWIRRAPGQGPEWMGWM | CTGTHNLVSWVQHQPGRAP |
| | NPMGGQVNIPWKFQGRVSMTRDTSIE | KLLIYDFNKRPSGVPDRFSGS |
| | TAFLDLRGLKSDDTAVYYCVRDRSN | GSGGTASLTITGLQDDDDAE |
| | GSGKRFESSNWFLDLWGRGTAVTIQS | YFCWAYEAFGGGTKLTVL |
| | (SEQ ID NO: 21) | (SEQ ID NO: 22) |
| Hydro2 | ADLVQSGAVVKKPGDSVRISCEAQG | QSALTQPRSVSASPGQSVTIS |
| | YRFPDYIIHWIRRAPGQGPEWMGWM | CTGTHNLVSWCQHQPGRAP |
| | NPMGGQVNIPQKFQGRVSMTRDTSIE | KLLIYDFNKRPSGVPDRFSGS |
| | TAFLDLRGLKSDDTAVYYCVRDRSN | GSGGTASLTITGLQDDDDAE |
| | GSGKRFESSNWFLDLWGRGTAVTIQS | YFCWAYENFGGGTKLTVL |
| | (SEQ ID NO: 23) | (SEQ ID NO: 24) |
| Germ12_NglycoSA_ | QVDLVQSGAVVKKPGDSVRVSCEAQ | QSALTQPRSVSGSPGQSVTIS |
| CysCA | GYRFPDYIIHWIRRAPGQGPEWMGW | CTGTHNLVSWAQHQPGRAP |
| | MNPMGGQVNIPWKFQGRVSMTRDTS | KLLIYDFNKRPSGVPDRFSGS |
| | IETAYMDLRGLRSDDTAVYYCVRDR | GSGGTASLTISGLQDEDDAE |
| | SNGAGKRFESSNWFLDLWGRGTLVT | YFCWAYEAFGGGTKLTVL |
| | VSS (SEQ ID NO: 25) | (SEQ ID NO: 26) |
| Germ12_NglycoSA_ | QVDLVQSGAVVKKPGDSVRVSCEAQ | QSALTQPRSVSGSPGQSVTIS |
| CysCV | GYRFPDYIIHWIRRAPGQGPEWMGW | CTGTHNLVSWVQHQPGRAP |
| | MNPMGGQVNIPWKFQGRVSMTRDTS | KLLIYDFNKRPSGVPDRFSGS |
| | IETAYMDLRGLRSDDTAVYYCVRDR | GSGGTASLTISGLQDEDDAE |
| | SNGAGKRFESSNWFLDLWGRGTLVT | YFCWAYEAFGGGTKLTVL |
| | VSS (SEQ ID NO: 27) | (SEQ ID NO: 28) |
| Germ23_NglycoSA_ | QVQLVQSGAEVKKPGASVKVSCEAS | QSALTQPRSVSGSPGQSVTIS |
| CysCV | GYRFPDYIIHWIRRAPGQGPEWMGW | CTGTHNLVSWVQHHPGKAP |
| | MNPMGGQVNIPWKFQGRVSMTRDTS | KLMIYDFNKRPSGVPDRFSG |
| | IETAYMELRGLRSDDTAVYYCVRDRS | SGSGGTASLTISGLQAEDEAE |
| | NGAGKRFESSNWFLDLWGRGTLVTV | YFCWAYEAFGGGTKLTVL |
| | SS (SEQ ID NO: 35) | (SEQ ID NO: 36) |
| Cd4bs_H53MS_ | ADLVQSGAVVKKPGDSVRISCEAQG | QSALTQPRSVSASPGQSVTIS |
| H54GY | YRFPDYIIHWIRRAPGQGPEWMGWM | CTGTHNLVSWCQHQPGRAP |
| | NPSYGQVNIPWKFQGRVSMTRDTSIE | KLLIYDFNKRPSGVPDRFSGS |
| | TAFLDLRGLKSDDTAVYYCVRDRSN | GSGGTASLTITGLQDDDDAE |
| | GSGKRFESSNWFLDLWGRGTAVTIQS | YFCWAYEAFGGGTKLTVL |
| | (SEQ ID NO: 57) | (SEQ ID NO: 58) |

TABLE 12

| Germ12_NglycoSA_ | QVDLVQSGAVVKKPGDSVRVSC | QSALTQPRSVSGSPGQSV |
|---|---|---|
| CysCV | EAQGYRFPDYIIHWIRRAPGQGP | TISCTGTHNLVSWVQHQP |
| | EWMGWMNPMGGQVNIPWKFQ | GRAPKLLIYDFNKRPSGV |
| | GRVSMTRDTSIETAYMDLRGLRS | PDRFSGSGSGGTASLTISG |
| | DDTAVYYCVRDRSNGAGKRFES | LQDEDDAEYFCWAYEAF |
| | SNWFLDLWGRGTLVTVSS (SEQ | GGGTKLTVL (SEQ ID NO: |
| | ID NO: 27) | 28) |
| Germ18_NglycoSA_ | QVQLVQSGAEVKKPGASVKVSC | QSALTQPRSVSGSPGQSV |
| CysCV | EASGYRFPDYIIHWIRRAPGQGPE | TISCTGTHNLVSWVQHQP |
| | WMGWMNPMGGQVNIPWKFQG | GRAPKLLIYDFNKRPSGV |
| | RVSMTRDTSIETAYMELRGLRSD | PDRFSGSGSGGTASLTISG |
| | DTAVYYCVRDRSNGAGKRFESS | LQDEDDAEYFCWAYEAF |
| | NWFLDLWGRGTLVTVSS (SEQ | GGGTKLTVL (SEQ ID NO: |
| | ID NO: 29) | 30) |
| Germ17_NglycoSA_ | QVDLVQSGAVVKKPGDSVRVSC | QSALTQPRSVSGSPGQSV |
| CysCA | EAQGYRFPDYIIHWIRRAPGQGP | TISCTGTHNLVSWAQHHP |
| | EWMGWMNPMGGQVNIPWKFQ | GKAPKLMIYDFNKRPSG |
| | GRVSMTRDTSIETAYMDLRGLRS | VPDRFSGSGSGGTASLTIS |
| | DDTAVYYCVRDRSNGAGKRFES | GLQAEDEAEYFCWAYEA |
| | SNWFLDLWGRGTLVTVSS (SEQ | FGGGTKLTVL (SEQ ID |
| | ID NO: 31) | NO: 32) |
| Germ17_NglycoSA_ | QVDLVQSGAVVKKPGDSVRVSC | QSALTQPRSVSGSPGQSV |
| CysCV | EAQGYRFPDYIIHWIRRAPGQGP | TISCTGTHNLVSWVQHHP |
| | EWMGWMNPMGGQVNIPWKFQ | GKAPKLMIYDFNKRPSG |
| | GRVSMTRDTSIETAYMDLRGLRS | VPDRFSGSGSGGTASLTIS |
| | DDTAVYYCVRDRSNGAGKRFES | GLQAEDEAEYFCWAYEA |
| | SNWFLDLWGRGTLVTVSS (SEQ | FGGGTKLTVL (SEQ ID |
| | ID NO: 33) | NO: 34) |
| Germ23_NglycoSA_ | QVQLVQSGAEVKKPGASVKVSC | QSALTQPRSVSGSPGQSV |
| CysCV | EASGYRFPDYIIHWIRRAPGQGPE | TISCTGTHNLVSWVQHHP |
| | WMGWMNPMGGQVNIPWKFQG | GKAPKLMIYDFNKRPSG |
| | RVSMTRDTSIETAYMELRGLRSD | VPDRFSGSGSGGTASLTIS |
| | DTAVYYCVRDRSNGAGKRFESS | GLQAEDEAEYFCWAYEA |
| | NWFLDLWGRGTLVTVSS (SEQ | FGGGTKLTVL (SEQ ID |
| | ID NO: 35) | NO: 36) |
| Germ12_NglycoSA_ | QVDLVQSGAVVKKPGDSVRVSC | QSALTQPRSVSGSPGQSV |
| CysCV_H61WQ | EAQGYRFPDYIIHWIRRAPGQGP | TISCTGTHNLVSWVQHQP |
| | EWMGWMNPMGGQVNIPQKFQG | GRAPKLLIYDFNKRPSGV |
| | RVSMTRDTSIETAYMDLRGLRSD | PDRFSGSGSGGTASLTISG |
| | DTAVYYCVRDRSNGAGKRFESS | LQDEDDAEYFCWAYEAF |
| | NWFLDLWGRGTLVTVSS (SEQ | GGGTKLTVL (SEQ ID NO: |
| | ID NO: 37) | 38) |
| Germ12_NglycoSA_ | QVDLVQSGAVVKKPGDSVRVSC | QSALTQPRSVSGSPGQSV |
| CysCV_L89AN | EAQGYRFPDYIIHWIRRAPGQGP | TISCTGTHNLVSWVQHQP |
| | EWMGWMNPMGGQVNIPWKFQ | GRAPKLLIYDFNKRPSGV |
| | GRVSMTRDTSIETAYMDLRGLRS | PDRFSGSGSGGTASLTISG |
| | DDTAVYYCVRDRSNGAGKRFES | LQDEDDAEYFCWAYENF |
| | SNWFLDLWGRGTLVTVSS (SEQ | GGGTKLTVL (SEQ ID NO: |
| | ID NO: 39) | 40) |
| Germ12_NglycoSA_ | QVDLVQSGAVVKKPGDSVRVSC | QSALTQPRSVSGSPGQSV |
| CysCV_H61WY | EAQGYRFPDYIIHWIRRAPGQGP | TISCTGTHNLVSWVQHQP |
| | EWMGWMNPMGGQVNIPYKFQG | GRAPKLLIYDFNKRPSGV |
| | RVSMTRDTSIETAYMDLRGLRSD | PDRFSGSGSGGTASLTISG |
| | DTAVYYCVRDRSNGAGKRFESS | LQDEDDAEYFCWAYEAF |
| | NWFLDLWGRGTLVTVSS (SEQ | GGGTKLTVL (SEQ ID NO: |
| | ID NO: 41) | 42) |
| Germ12_NglycoSA_ | QVDLVQSGAVVKKPGDSVRVSC | QSALTQPRSVSGSPGQSV |
| CysCV_H61WH | EAQGYRFPDYIIHWIRRAPGQGP | TISCTGTHNLVSWVQHQP |
| | EWMGWMNPMGGQVNIPHKFQG | GRAPKLLIYDFNKRPSGV |
| | RVSMTRDTSIETAYMDLRGLRSD | PDRFSGSGSGGTASLTISG |
| | DTAVYYCVRDRSNGAGKRFESS | LQDEDDAEYFCWAYEAF |
| | NWFLDLWGRGTLVTVSS (SEQ | GGGTKLTVL (SEQ ID NO: |
| | ID NO: 43) | 44) |
| Germ12_NglycoSA_ | QVDLVQSGAVVKKPGDSVRVSC | QSALTQPRSVSGSPGQSV |
| CysCV_H61WR | EAQGYRFPDYIIHWIRRAPGQGP | TISCTGTHNLVSWVQHQP |
| | EWMGWMNPMGGQVNIPRKFQG | GRAPKLLIYDFNKRPSGV |
| | RVSMTRDTSIETAYMDLRGLRSD | PDRFSGSGSGGTASLTISG |
| | DTAVYYCVRDRSNGAGKRFESS | LQDEDDAEYFCWAYEAF |
| | NWFLDLWGRGTLVTVSS (SEQ | GGGTKLTVL (SEQ ID NO: |
| | ID NO: 71) | 72) |
| Germ12_NglycoSA_ | QVDLVQSGAVVKKPGDSVRVSC | QSALTQPRSVSGSPGQSV |
| CysCV_H107FY | EAQGYRFPDYIIHWIRRAPGQGP | TISCTGTHNLVSWVQHQP |
| | EWMGWMNPMGGQVNIPWKFQ | GRAPKLLIYDFNKRPSGV |
| | GRVSMTRDTSIETAYMDLRGLRS | PDRFSGSGSGGTASLTISG |
| | DDTAVYYCVRDRSNGAGKRYES | LQDEDDAEYFCWAYEAF |
| | SNWFLDLWGRGTLVTVSS (SEQ | GGGTKLTVL (SEQ ID NO: |
| | ID NO: 47) | 48) |
| Germ12_NglycoSA_ | QVDLVQSGAVVKKPGDSVRVSC | QSALTQPRSVSGSPGQSV |
| CysCV_L89AN_H61WY | EAQGYRFPDYIIHWIRRAPGQGP | TISCTGTHNLVSWVQHQP |
| | EWMGWMNPMGGQVNIPYKFQG | GRAPKLLIYDFNKRPSGV |
| | RVSMTRDTSIETAYMDLRGLRSD | PDRFSGSGSGGTASLTISG |
| | DTAVYYCVRDRSNGAGKRFESS | LQDEDDAEYFCWAYENF |
| | NWFLDLWGRGTLVTVSS (SEQ | GGGTKLTVL (SEQ ID NO: |
| | ID NO: 49) | 50) |
| Germ12_NglycoSA_ | QVDLVQSGAVVKKPGDSVRVSC | QSALTQPRSVSGSPGQSV |
| CysCV_L89AN_H107FY | EAQGYRFPDYIIHWIRRAPGQGP | TISCTGTHNLVSWVQHQP |
| | EWMGWMNPMGGQVNIPWKFQ | GRAPKLLIYDFNKRPSGV |
| | GRVSMTRDTSIETAYMDLRGLRS | PDRFSGSGSGGTASLTISG |
| | DDTAVYYCVRDRSNGAGKRYES | LQDEDDAEYFCWAYENF |
| | SNWFLDLWGRGTLVTVSS (SEQ | GGGTKLTVL (SEQ ID NO: |
| | ID NO: 51) | 52) |
| Germ12_NglycoSA_ | QVDLVQSGAVVKKPGDSVRVSC | QSALTQPRSVSGSPGQSV |
| CysCV_H61WY_H107FY | EAQGYRFPDYIIHWIRRAPGQGP | TISCTGTHNLVSWVQHQP |
| | EWMGWMNPMGGQVNIPYKFQG | GRAPKLLIYDFNKRPSGV |
| | RVSMTRDTSIETAYMDLRGLRSD | PDRFSGSGSGGTASLTISG |
| | DTAVYYCVRDRSNGAGKRYESS | LQDEDDAEYFCWAYEAF |
| | NWFLDLWGRGTLVTVSS (SEQ | GGGTKLTVL (SEQ ID NO: |
| | ID NO: 73) | 74) |
| Germ12_NglycoSA_ | QVDLVQSGAVVKKPGDSVRVSC | QSALTQPRSVSGSPGQSV |
| CysCV_L89AN_H61WY_ | EAQGYRFPDYIIHWIRRAPGQGP | TISCTGTHNLVSWVQHQP |
| H107FY | EWMGWMNPMGGQVNIPYKFQG | GRAPKLLIYDFNKRPSGV |
| | RVSMTRDTSIETAYMDLRGLRSD | PDRFSGSGSGGTASLTISG |
| | DTAVYYCVRDRSNGAGKRYESS | LQDEDDAEYFCWAYENF |
| | NWFLDLWGRGTLVTVSS (SEQ | GGGTKLTVL (SEQ ID NO: |
| | ID NO: 55) | 56) |
| Cd4bs_H53MS_H54GY | ADLVQSGAVVKKPGDSVRISCE | QSALTQPRSVSASPGQSV |
| | AQGYRFPDYIIHWIRRAPGQGPE | TISCTGTHNLVSWCQHQP |
| | WMGWMNPSYGQVNIPWKFQGR | GRAPKLLIYDFNKRPSGV |
| | VSMTRDTSIETAFLDLRGLKSDD | PDRFSGSGSGGTASLTITG |
| | TAVYYCVRDRSNGSGKRFESSN | LQDDDDAEYFCWAYEAF |
| | WFLDLWGRGTAVTIQS (SEQ ID | GGGTKLTVL (SEQ ID NO: |
| | NO: 57) | 58) |

TABLE 13
Positions in the V_{H} and V_{L} regions of L1A2 that
can be varied to address potential liabilities.

| | | | | | | Mutation |
|---|---|---|---|---|---|---|
| | | | | Risk of | Change | in an L1A1 |
| | | | | leaving | in | or L1A4 |
| Parent | Mutation | Purpose(s) | Location | as native | charge | sibling ? |
| L1A2 | H0XQ | germlining | H-FW1 | medium | 0.0 | no |
| L1A2 | H1AV | germlining | H-FW1 | medium | 0.0 | no |
| L1A2 | H2DQ | germlining | H-FW1 | medium | +1.0 | no |
| L1A2 | H9VE | germlining | H-FW1 | medium | −1.0 | no |
| L1A2 | H15DA | germlining | H-FW1 | medium | +1.0 | yes |
| L1A2 | H18RK | germlining | H-FW1 | medium | 0.0 | yes |
| L1A2 | H19IV | germlining | H-FW1 | medium | 0.0 | yes |
| L1A2 | H22EK | germlining | H-FW1 | medium | +2.0 | no |
| L1A2 | H24QS | germlining | H-FW1 | medium | 0.0 | no |
| L1A2 | H36IV | germlining | H-FW2 | medium | 0.0 | yes |
| L1A2 | H38RQ | germlining | H-FW2 | medium | −1.0 | yes |
| L1A2 | H44PL | germlining | H-FW2 | medium | 0.0 | yes |
| L1A2 | H49WY | liab: Trp | H-CDR2 | low | 0.0 | no |
| | | oxidation | | | | |
| L1A2 | H49WF | liab: Trp | H-CDR2 | low | 0.0 | no |
| | | oxidation | | | | |
| L1A2 | H50MI | liab: Met | H-CDR2 | low | 0.0 | no |
| | | oxidation | | | | |
| L1A2 | H50MQ | liab: Met | H-CDR2 | low | 0.0 | no |
| | | oxidation | | | | |
| L1A2 | H50ML | liab: Met | H-CDR2 | low | 0.0 | no |
| | | oxidation | | | | |
| L1A2 | H50MS | liab: Met | H-CDR2 | low | 0.0 | no |
| | | oxidation | | | | |
| L1A2 | H50MA | liab: Met | H-CDR2 | low | 0.0 | no |
| | | oxidation | | | | |
| L1A2 | H53MS | liab: Met | H-CDR2 | low | 0.0 | yes |
| | | oxidation | | | | |
| L1A2 | H53MV | liab: Met | H-CDR2 | low | 0.0 | yes |
| | | oxidation | | | | |
| L1A2 | H53MQ | liab: Met | H-CDR2 | low | 0.0 | no |
| | | oxidation | | | | |
| L1A2 | H53ML | liab: Met | H-CDR2 | low | 0.0 | no |
| | | oxidation | | | | |
| L1A2 | H53MA | liab: Met | H-CDR2 | low | 0.0 | no |
| | | oxidation | | | | |
| L1A2 | H61WQ | liab: Trp | H-CDR2 | medium | 0.0 | yes |
| | | oxidation & | | | | |
| | | surface | | | | |
| | | hydrophobic | | | | |
| L1A2 | H61WY | liab: Trp | H-CDR2 | medium | 0.0 | no |
| | | oxidation & | | | | |
| | | surface | | | | |
| | | hydrophobic | | | | |
| L1A2 | H61WF | liab: Trp | H-CDR2 | medium | 0.0 | no |
| | | oxidation | | | | |
| L1A2 | H62KN | liab: Lys | H-CDR2 | low | −1.0 | yes |
| | | glycation | | | | |
| L1A2 | H62KR | liab: Lys | H-CDR2 | low | 0.0 | no |
| | | glycation | | | | |
| L1A2 | H62KQ | liab: Lys | H-CDR2 | low | −1.0 | no |
| | | glycation | | | | |
| L1A2 | H62KS | liab: Lys | H-CDR2 | low | −1.0 | no |
| | | glycation | | | | |
| L1A2 | H62KA | liab: Lys | H-CDR2 | low | −1.0 | no |
| | | glycation | | | | |
| L1A2 | H68ST | germlining | H-FW3 | medium | 0.0 | yes |
| L1A2 | H75IT | liab: surface | H-FW3 | medium | 0.0 | yes |
| | | hydrophobic | | | | |
| L1A2 | H76ES | germlining | H-FW3 | medium | +1.0 | no |
| L1A2 | H79FY | germlining | H-FW3 | medium | 0.0 | yes |
| L1A2 | H80LM | germlining | H-FW3 | medium | 0.0 | no |
| L1A2 | H81DE | germlining | H-FW3 | medium | 0.0 | yes |
| L1A2 | H83RS | germlining | H-FW3 | medium | −1.0 | yes |
| L1A2 | H84GR | germlining | H-FW3 | medium | +1.0 | no |
| L1A2 | H86KR | germlining | H-FW3 | medium | 0.0 | yes |
| L1A2 | H101ND | liab: Nglyco | H-CDR3 | high | −1.0 | yes |
| L1A2 | H101NS | liab: Nglyco, NG | H-CDR3 | high | 0.0 | no |
| | | deamidation | | | | |
| L1A2 | H101NA | liab: Nglyco, NG | H-CDR3 | high | 0.0 | no |
| | | deamidation | | | | |
| L1A2 | H101NQ | liab: Nglyco, NG | H-CDR3 | high | 0.0 | no |
| | | deamidation | | | | |
| L1A2 | H103SW | liab: Nglyco | H-CDR3 | high | 0.0 | no |
| L1A2 | H103SA | liab: Nglyco | H-CDR3 | high | 0.0 | no |
| L1A2 | H103SN | liab: Nglyco | H-CDR3 | high | 0.0 | no |
| L1A2 | H105KQ | liab: proteolysis | H-CDR3 | medium | −1.0 | no |
| L1A2 | H105KS | liab: proteolysis | H-CDR3 | medium | −1.0 | no |
| L1A2 | H105KA | liab: proteolysis | H-CDR3 | medium | −1.0 | no |
| L1A2 | H106RQ | liab: proteolysis | H-CDR3 | medium | −1.0 | no |
| L1A2 | H106RS | liab: proteolysis | H-CDR3 | medium | −1.0 | no |
| L1A2 | H106RA | liab: proteolysis | H-CDR3 | medium | −1.0 | no |
| L1A2 | H107FY | liab: surface | H-CDR3 | medium | 0.0 | no |
| | | hydrophobic | | | | |
| L1A2 | H112WY | liab: Trp | H-CDR3 | low | 0.0 | no |
| | | oxidation | | | | |
| L1A2 | H112WF | liab: Trp | H-CDR3 | low | 0.0 | no |
| | | oxidation | | | | |
| L1A2 | H122AL | germlining | H-FW4 | medium | 0.0 | no |
| L1A2 | H125IV | germlining | H-FW4 | medium | 0.0 | yes |
| L1A2 | H126QS | germlining | H-FW4 | medium | 0.0 | no |
| L1A2 | L12AG | germlining | L-FW1 | medium | 0.0 | yes |
| L1A2 | L28LY | liab: surface | L-CDR1 | medium | 0.0 | yes |
| | | hydrophobic | | | | |
| L1A2 | L32CY | liab: cys & | L-FW2 | high | 0.0 | no |
| | | germlining | | | | |
| L1A2 | L32CA | liab: cys | L-FW2 | high | 0.0 | no |
| L1A2 | L32CV | liab: cys | L-FW2 | high | 0.0 | no |
| L1A2 | L32CL | liab: cys | L-FW2 | high | 0.0 | no |
| L1A2 | L32CI | liab: cys | L-FW2 | high | 0.0 | no |
| L1A2 | L34HQ | germlining | L-FW2 | medium | −0.5 | yes |
| L1A2 | L35QH | germlining | L-FW2 | medium | +0.5 | yes |
| L1A2 | L38RK | germlining | L-FW2 | medium | 0.0 | no |
| L1A2 | L43LM | germlining | L-FW2 | medium | 0.0 | no |
| L1A2 | L49KQ | liab: proteolysis | L-CDR2 | medium | −1.0 | no |
| L1A2 | L49KS | liab: proteolysis | L-CDR2 | medium | −1.0 | no |
| L1A2 | L49KA | liab: proteolysis | L-CDR2 | medium | −1.0 | no |
| L1A2 | L50RQ | liab: proteolysis | L-CDR2 | medium | −1.0 | no |
| L1A2 | L50RS | liab: proteolysis | L-CDR2 | medium | −1.0 | no |
| L1A2 | L50RA | liab: proteolysis | L-CDR2 | medium | −1.0 | no |
| L1A2 | L62GK | germlining | L-FW3 | medium | +1.0 | no |
| L1A2 | L65GN | germlining | L-FW3 | medium | 0.0 | yes |
| L1A2 | L72TS | germlining | L-FW3 | medium | 0.0 | yes |
| L1A2 | L76DA | germlining | L-FW3 | medium | +1.0 | yes |
| L1A2 | L77DE | germlining | L-FW3 | medium | 0.0 | yes |
| L1A2 | L79DE | germlining | L-FW3 | medium | 0.0 | yes |
| L1A2 | L81ED | germlining | L-FW3 | medium | 0.0 | yes |
| L1A2 | L83FY | germlining | L-FW3 | medium | 0.0 | no |
| L1A2 | L85WY | liab: Trp | L-CDR3 | low | 0.0 | no |
| | | oxidation | | | | |
| L1A2 | L85WF | liab: Trp | L-CDR3 | low | 0.0 | no |
| | | oxidation | | | | |
| L1A2 | L89AN | liab: surface | L-CDR3 | medium | 0.0 | yes |
| | | hydrophobic | | | | |

### Example 2: Binding Analysis of Variant Antibodies

Binding analyses were performed for twelve of the fifteen variant antibodies as shown in Table 11 having substitutions as summarized in Table 10.

To assess whether antibody modifications affected binding strength, binding affinity for the twelve variants was measured using biolayer interferometry (BLI) against two HIV envelopes (monomeric gp120 protein): a Clade B protein, BaL, and a Clade C protein, DU172.17. The variants were expressed from HEK-293 cells using transient transfection and purified with protein A affinity chromatography. The parent L1 A2 antibody produced at the same time and with the same methods was also evaluated. A second lot of L1A2 was produced at a different contract research organization (CRO) using similar transient HEK-293 expression and protein A affinity chromatography purification and tested in parallel to assess production consistency. Two aliquots of this second lot of L1A2 were tested to assess assay reproducibility. We also tested a fourth aliquot of L1A2 expressed from CHO cells (and purified by protein A affinity chromatography) to determine whether production in a different cell line might affect binding affinity. Binding of 3BNC117, a well-characterized CD4-binding site antibody, was also evaluated as a positive control as well as a CRO-selected negative control.

Binding was evaluated using an Octet HTX (Pall ForteBio) at 25° C. Purified human antibodies diluted to 2 ug/mL were loaded onto anti-human IgG Fc capture biosensors. Loaded sensors were dipped into a three-fold dilution series of either Clade B BaL gp120 protein or Clade C DU172.17 gp120 protein (Immune Technology Corp.) starting at 500 nM. Kinetic constants were calculated using a monovalent (1:1) binding model. Binding data for the 12 variants are provided in Table 14. In this analysis, most of the variant antibodies exhibited K_{D} values within two-fold of the K_{D} value for the L1A2 average.

TABLE 14

| | | KD | | Kon | Koff |
|---|---|---|---|---|---|
| Name | Purpose | (nM) | fold | (1/Ms) | (1/s) |
| | | BaL Octet | | | |
| Germ6 | Germline | 0.92 | 1.70 | 7.71E+04 | 7.11E−05 |
| Germ12 | Germline | 0.88 | 1.62 | 8.08E+04 | 7.09E−05 |
| Germ18 | Germline | 0.98 | 1.81 | 7.22E+04 | 7.09E−05 |
| Germ17 | Germline | 0.65 | 1.20 | 7.93E+04 | 5.15E−05 |
| Germ23 | Germline | 0.97 | 1.79 | 7.91E+04 | 7.66E−05 |
| NglycoSA | Remove Nglyco | 0.84 | 1.54 | 7.33E+04 | 6.12E−05 |
| NglycoND | Remove Nglyco | 18.70 | 34.50 | 6.84E+04 | 1.28E−03 |
| NglycoNDplus6 | Remove Nglyco | 11.30 | 20.85 | 5.33E+04 | 6.00E−04 |
| CysCA | Remove Cys | 1.10 | 2.03 | 7.19E+04 | 8.01E−05 |
| CysCV | Remove Cys | 0.82 | 1.52 | 7.79E+04 | 6.40E−05 |
| Hydro2 | Reduce hydrophobic surface | 0.96 | 1.77 | 7.47E+04 | 7.18E−05 |
| Germ12_NglycoSA_CysCA | Germline + Remove Nglyco & | 0.63 | 1.16 | 7.83E+04 | 4.92E−05 |
| | Cys | | | | |
| L1A2 | Test parent L1A2 | 0.44 | 0.81 | 8.91E+04 | 3.91E−05 |
| L1A2 (CRO #2) - run1 | Test inter-CRO production | 0.77 | 1.42 | 7.82E+04 | 6.02E−05 |
| L1A2 (CRO #2) - run2 | Test assay reproducibility | 0.66 | 1.22 | 8.21E+04 | 5.42E−05 |
| L1A2 (made in CHO) | Test CHO versus HEK production | 0.30 | 0.55 | 7.58E+04 | 2.27E−05 |
| Average of 4 runs of L1A2 | | 0.54 | 1.00 | 8.13E+04 | 4.41E−05 |
| 3BNC117 | | 3.29 | 6.07 | 8.85E+04 | 2.91E−04 |
| | | DU172 Octet | | | |
| Germ6 | Germline | 19.30 | 0.76 | 2.41E+04 | 4.66E−04 |
| Germ12 | Germline | 21.50 | 0.85 | 2.04E+04 | 4.38E−04 |
| Germ18 | Germline | 21.30 | 0.84 | 1.95E+04 | 4.15E−04 |
| Germ17 | Germline | 17.50 | 0.69 | 1.89E+04 | 3.31E−04 |
| Germ23 | Germline | 22.40 | 0.88 | 1.64E+04 | 3.68E−04 |
| NglycoSA | Remove Nglyco | 18.40 | 0.72 | 1.65E+04 | 3.03E−04 |
| NglycoND | Remove Nglyco | 368.00 | 14.47 | 4.97E+04 | 1.83E−02 |
| NglycoNDplus6 | Remove Nglyco | N/A | N/A | No | No |
| | | | | binding | binding |
| CysCA | Remove Cys | 19.60 | 0.77 | 2.28E+04 | 4.46E−04 |
| CysCV | Remove Cys | 26.70 | 1.05 | 1.85E+04 | 4.95E−04 |
| Hydro2 | Reduce hydrophobic surface | 55.50 | 2.18 | 1.76E+04 | 9.74E−04 |
| Germ12_NglycoSA_CysCA | Germline + Remove Nglyco & | 18.10 | 0.71 | 1.86E+04 | 3.37E−04 |
| | Cys | | | | |
| L1A2 | Test parent L1A2 | 21.20 | 0.83 | 2.06E+04 | 4.37E−04 |
| L1A2 (CRO #2) - run1 | Test inter-CRO production | 25.20 | 0.99 | 2.27E+04 | 5.70E−04 |
| L1A2 (CRO #2) - run2 | Test assay reproducibility | 35.00 | 1.38 | 1.81E+04 | 6.34E−04 |
| L1A2 (made in CHO) | Test CHO versus HEK production | 20.30 | 0.80 | 2.28E+04 | 4.64E−04 |
| Average of 4 runs of L1A2 | | 25.43 | 1.00 | 2.11E+04 | 5.26E−04 |
| 3BNC117 | | 95.80 | 3.77 | 4.77E+04 | 4.57E−03 |

Comparison of affinity measured using the two L1A2 aliquots from the same production run demonstrated strong intra-assay reproducibility, as the measurements differed by only 17% (BaL) and 39% (DU172.17). Comparison of affinity measured using all four L1 A2 aliquots demonstrated strong inter-CRO and inter-cell line reproducibility against both proteins, with a 2.6 fold range measured against BaL (average: 0.54 nM; range: 0.30-0.77 nM) and a 1.7 fold range measured against DU172.17 (average: 25.4; range: 20.3-35.0 nM).

Of the 12 variants evaluated, 5 focused on germlining modifications that mutated variable region framework residues to the residue encoded by the predicted germline gene of origin (Germ6, Germ12, Germ18, Germ17, and Germ23). These modifications were introduced to reduce potential immunogenicity risk in humans. The 5 variants contained from 6 to 23 mutations that ranged from a relatively low to medium predicted risk of deleteriously affecting antibody binding. Binding appeared to be slightly weaker for all 5 germlined variants when measured against BaL, but the values are likely within assay error, and all were within 2-fold of the mean of the four L1A2 measurements. When binding was assessed using DU172.17, binding affinity appeared slightly stronger for all 5 germlined variants, but the values again are likely within assay error, and all were within 2-fold of the L1A2 mean. Thus, these data suggest that incorporation of up to at least 23 germlining modifications can be accommodated with minimal impact on binding affinity.

A second set of variants evaluated contained modifications that disrupted the consensus N-linked glycosylation sequence motif in H-CDR3 (H101N-H102G-H103S). An N-linked glycosylation motif may be glycosylated during expression in eukaryotic cells, such as HEK293 or CHO, and the glycosylated antibody may exhibit decreased potency, poor in vivo pharmokinetics, lower yield, increased heterogeneity, purification complications, batch-to-batch reproducibility issues, or other manufacturing consistency issues. Lack of glycosylation in one cell line (e.g., HEK293, typically used in research-stage production) does not necessarily mean that another cell line will not glycosylate (e.g., CHO, typically used in clinical-stage manufacturing). Three approaches to disrupting the consensus N-linked glycosylation motif were tested: “NglycoSA”, which mutated the serine to an alanine; “NglycoND”, which mutated the asparagine to an aspartic acid; and “NglycoNDplus6, which mutated the asparagine to an aspartic acid and also mutated six more heavy chain CDR positions (H27RT, H100SG, H104GR, H105KR, H106RH, H108ED, relative to SEQ ID NO:1). NglycoSA demonstrated comparable binding affinity to both proteins. However, both variants that comprised the H103ND mutation demonstrated a 21-34 fold loss in binding against BaL. When binding was tested against DU172.17, NglycoND activity was reduced 14-fold, while NglycoNDplus6 could no longer bind to the protein at the concentrations tested. Reductions in binding affinity were largely driven by faster off rates.

Three additional variants were tested that contained modifications intended to improve protein developability and manufacturability, including elimination of a cysteine in L-FW2 and reduction of the antibody's hydrophobic surface. A free cysteine can lead to decreased production yield, increased product heterogeneity, decreased product stability, increased aggregation, or decreased activity. Hydrophobic surface can lead to increased self-association, increased aggregation, increased viscosity, decreased stability, increased nonspecific interactions, or poor in vivo pharmacokinetics. Two different mutations to remove the L-FW2 cysteine were tested: cysteine to alanine, “CysCA”, and cysteine to valine, “CysCV”. When tested against BaL, both demonstrated slightly weaker binding as compared to the parent L1A2 binding, but the CysCV difference is likely within assay error and is within 2-fold of L1A2. In contrast, both variants appeared to bind comparably (CysCV) or slightly more strongly (CysCA) than parent L1A2 to DU172.17. Lastly, two separate modifications, L89AN and H61WQ, were combined together as “Hydro2” to reduce the antibody's hydrophobic surface. Hydro2 demonstrated slightly, though not significantly, weaker binding against both proteins.

The twelfth variant evaluated combined 14 mutations into one antibody, “Germ12_NglycoSA_CysCA”, comprising: the 12 germlining mutations as made in “Germ12”, the N-glycosylation motif mutation H103SA as made in “NglycoSA”, and the cysteine mutation L32CA as made in “CysCA”. Germ12_NglycoSA_CysCA demonstrated comparable binding affinity to both proteins (1.2-fold and 0.7-fold versus the L1A2 average, respectively), suggesting that such modifications can be combined together without significantly disrupting antibody binding.

H103SA is a more preferable mutation to remove the N-glycosylation motif in the HCDR3 as demonstrated by its preserved binding affinity and the >10-fold reduced binding affinity of the two H101ND-comprising variants. Other mutations, e.g., Gly, are also likely to preserve binding. In some embodiments, Gln, Asp, Glu, His, or Tyr may replace the serine at H103. In some embodiments, Asn may replace the serine at H103.

L32CV may be a desirable mutation to remove the L-FW2 cysteine, based on its modestly improved production yield (83 mg/L versus 49 mg/L, an increase of 69%), although this may be within the lot-to-lot production variability (which typically is approximately 2-fold). Both L32CV and L32CA retain binding. Other mutations at L32C may also preserve binding and/or improve production yield, such as cysteine to leucine or isoleucine.

All five germlined variants preserved binding. Germ12 does not increase net charge; however, Germ18, Germ17, and Germ23 only modestly increase net charge (+1 to +2.5). Germ17 exhibits the best binding affinities of the four, though they all can be within the experimental variability. Germ23 mutates the greatest number of framework positions to germline and thus exhibits the lowest predicted risk of potential immunogenicity in humans, although its production yield is modestly decreased (from 49 mg/L for L1A2 to 19 mg/L, a 2.6-fold reduction). Alternate combinations of germlining positions can also similarly retain binding while not exhibiting significant production yield decreases. Additional mutations, such as substitutions included in Table 7 and Table 8, can also retain binding affinity and yield.

Hydro2 exhibited a 2.2-fold loss of binding to DU172.17, though this difference is likely within range of experimental error. Alternative mutations to reduce potential hydrophobic surface include:

H61WQ by itself (e.g., if L89AN was the primary cause of the 2.2-fold binding loss to DU172.17);

L89AN by itself (e.g., if H61WQ was the primary cause of the 2.2-fold binding loss to DU172.17);

H61WY (a different way to reduce hydrophobicity at H61W: Tyr is still aromatic but more polar);

H61WH (a different way to reduce hydrophobicity at H61W: His is still aromatic but more polar);

H61WR (a different way to reduce hydrophobicity at H61W: Arg is very polar and seen in siblings);

H107FY (a different way to slightly reduce paratope hydrophobicity: Tyr adds a polar hydroxyl); and

Combinations of two or three of the above mutations across positions L89, H61, and H107.

The mutations described above may also be combined with other mutations described herein, including with other combinations, such as Germ12_NglycoSA_CysCA, Germ12_NglycoSA_CysCV, or Germ17_NglycoSA_CysCV. Additional examples of variant sequences incorporating substitutions as described in the Examples are shown in Table 12.

### Example 3: Neutralization Analysis of Variant Antibodies Evaluated Against a Panel of HIV-1 Viruses

Fourteen of the fifteen variant antibodies shown in Table 11 were evaluated for neutralization activity again a panel of 7 or 9-10 HIV-1 viruses (Table 15).

TABLE 15
Panel of viruses

| | HIV-1 | Included in |
|---|---|---|
| HIV-1 virus | virus | the 7-virus |
| name | clade | panel |
| AC10.0.29 | B | Y |
| THRO4156.18 | B | N |
| REJO4541.67 | B | Y |
| WITO4160.33 | B | N |
| DU172.17 | C | Y |
| ZM233M.PB6 | C | N |
| CAP45.2.00.G3 | C | Y |
| DU422.1 | C | Y |
| CNE20 | BC | Y |
| X1632_S2_B10 | G | Y |

Envelope (env) sequences were cloned into replication-competent infectious molecular clones (IMCs) carrying a Tat-regulated Renilla Luciferase reporter gene (Env.IMC.LucR). Antibodies were diluted to 50 μg/mL and then serially diluted 4-fold across 8 dilutions before being mixed with virus. The antibody-virus mixture was subsequently used to infected TZM-BL reporter cells. Reduction in luciferase expression was used to assess antibody-mediated viral neutralization. For each antibody dilution, neutralization was reported as the percent reduction in luciferase as compared to the virus-only negative control. Antibody neutralization titers were calculated using a sigmoidal dose response curve in Graphpad Prism and reported as the antibody concentration required to inhibit 50% of the viral infection (IC₅₀). Antibody IC₅₀ values calculated against viruses DU172.17, CAP45.2.00.G3, CNE20, REJO4541.67, X1632_S2_B10, AC10.0.29 and DU422.1 are derived from a single assay wherein each antibody was run in duplicate. These viruses are referred to as the “7-virus panel” (Table 15). Antibody IC₅₀ values calculated against viruses THRO4156.18, WITO4160.33 and ZM233M.PB6 are the average of two replicate assays where each antibody was run in duplicate. Some or all three of these three additional viruses combined with the 7-virus panel constitute the “expanded virus panel”.

To assess whether modifications to L1A2 affected neutralization activity, we measured IC₅₀ values for the 14 variants against a panel of 7-10 HIV-1 IMCs with a range of previously reported sensitivity to the parent (L1A2) and control (3BNC117) antibodies. All 14 variants were expressed from HEK-293 cells using transient transfection and purified with protein A affinity chromatography. In addition to these variants, we also tested the parent antibody L1A2 produced using the same methods. A second lot of L1A2 was produced at a different CRO (using similar transient HEK-293 expression and protein A affinity chromatography purification) and tested in parallel to assess production consistency between CROs. We also tested a third aliquot of L1A2 expressed from CHO cells and purified by protein A affinity chromatography to determine whether production in a different cell line might affect antibody neutralization activity. In addition to these 17 antibodies, we also tested 3BNC117, a well-characterized CD4-binding site antibody as a positive control and a non-HIV specific human IgG1 antibody as a negative control.

To assess inter-CRO and inter-cell line reproducibility, we compared the IC₅₀ geometric mean and breadth for the three L1A2 lots evaluated against a panel of 7 IMCs. The three lots demonstrated strong inter-lot reproducibility; the geometric mean values ranged from 0.114 μg/mL to 0.151 μg/mL and breadth was 57% for all three viruses. Further, the largest fold difference in IC₅₀ values between any 2 mAbs was <2-fold (Table 16).

Of the 14 variants, 5 focused on germlining modifications that mutated variable region framework residues to the residue encoded by the predicted germline gene of origin (Germ6, Germ12, Germ18, Germ17, and Germ23). These modifications were introduced to reduce potential immunogenicity risk in humans. The 5 variants comprised 6-23 mutations. All five variants were evaluated against the same panel of 7 HIV-1 IMCs. Geometric mean IC₅₀ values ranged between 0.058 μg/mL and 0.140 μg/mL, suggesting that these variants were comparably potent to parent antibody L1A2 (geometric mean=0.151 μg/mL). Each of the 5 variant antibodies neutralized the same 4 IMCs as the parent antibody L1A2, thus resulting in identical breadth scores (57%). The largest fold difference in IC₅₀ values between any two germline'd variants or the parent L1A2 antibody was 5.97 (IC₅₀ range: 0.035 μg/mL to 0.209 μg/mL) as reported against HIV-1 virus CAP45.2.00.G3. This difference is likely within experimental variation. In conclusion, the germlining modifications appear to have limited, if any, effects on L1A2 neutralization activity (Table 17).

The second set of modifications disrupted the consensus N-linked glycosylation sequence motif in H-CDR3 (H101N-H102G-H103S). Two approaches to disrupting the consensus N-linked glycosylation motif were tested: “NglycoSA”, which mutated the serine to an alanine, and “NglycoND”, which mutated the asparagine to an aspartic acid. NglycoSA demonstrated breadth equivalent to parent antibody L1A2 against both the 7-virus panel (57%) and the expanded virus panel (70%). The geometric mean IC₅₀ values for the parent L1A2 and NglycoSA variants are 0.151 μg/mL and 0.054 μg/mL, respectively, as calculated against the 7-virus panel, and 0.253 μg/mL and 0.111 μg/mL, respectively, as calculated against the expanded virus panel. Both antibodies demonstrate comparably potent neutralizing activity. In contrast, the NglycoND variant demonstrated reduced breadth against the 7-virus panel (43% vs 57% for parent L1A2 antibody) and modestly increased the geometric mean (0.444 μg/mL vs 0.151 μg/mL for parent L1A2 antibody). The increased geometric mean was driven largely by ˜65-fold increase in IC₅₀ values against HIV-1 virus DU172.17 as compared to parent mAb L1A2. In summary, modification of the N-linked glycosylation site by mutating the serine to an alanine had neither deleterious nor beneficial impacts on L1A2 neutralization activity. In contrast, modification of N-linked glycosylation site by mutating the asparagine to an aspartic acid significantly impaired neutralization activity against one virus and eliminated activity against a second virus at the highest antibody concentration evaluated in this study (Table 17). These neutralization data are consistent with reduced binding recorded for the “NglycoND” variant in Example 2.

Three variants comprised modifications intended to improve protein developability and manufacturability, including elimination of a cysteine in L-FW2 and reduction of the antibody's hydrophobic surface. Two different mutations to remove the L-FW2 cysteine were tested: cysteine to alanine, “CysCA”, and cysteine to valine, “CysCV”. A single variant, “Hydro2”, that combined two modifications, L89AN+H61WQ, to reduce surface hydrophobicity was also evaluated. All three variants exhibited similar breadth (57%) and potency as compared to parent mAb L1A2 when evaluated against the 7-virus panel. The geometric mean IC₅₀ values for the three variants ranged from 0.040 μg/mL to 0.047 μg/mL and are comparable to the geometric mean measured for L1A2 (0.151 μg/mL). The CysCV variant was also evaluated against the expanded virus panel. Consistent with data obtained from the 7-virus panel, both neutralizing activity and breadth were similar to parent mAb L1A2 (Table 17).

Three additional variants explored the combination of multiple mutations into one antibody. The first antibody, “Germ12_NglycoSA_CysCA”, comprised the 12 low-risk germlining mutations as made in “Germ12”, the N-glycosylation motif mutation H103SA as made in “NglycoSA”, and the cysteine mutation L32CA as made in “CysCA”. This antibody was previously evaluated for binding activity by Octet and the results described in Example 2. The second antibody, “Germ12_NglycoSA_CysCV” incorporated the same mutations except that it included the cysteine mutation L32CV instead of L32CA. The third antibody, “Germ23_NglycoSA_CysCV” incorporated the 23 low-risk germlining mutations as made in “Germ23”, the N-glycosylation motif mutation H103SA as made in “NglycoSA”, and the cysteine mutation L32CV as made in “CysCV”. All three variants demonstrated similarly potent neutralization activity (geometric mean IC₅₀ values ranged from 0.051 μg/mL to 0.085 μg/mL) as compared to parent antibody L1A2 (IC₅₀: 0.151 μg/mL) when evaluated against the 7-virus panel. All 4 antibodies also neutralized the same 4/7 viruses (breadth: 57%). Antibody “Germ12_NglycoSA_CysCV” was also evaluated against the 10-virus panel. Consistent with observations made against the 7-virus panel, both antibody potency (geometric mean IC₅₀ 0.288 μg/mL) and breadth were comparable to parent antibody L1A2 (geometric mean IC₅₀ 0.253 μg/mL). Taken together, these data suggest that mutations designed to reduce immunogenicity and eliminate a free cysteine and an N-linked glycosylation site can be combined into one molecule with limited impact on neutralization activity (Table 17).

Variant “Cd4bs_H53MS_H54GY” was designed to explore whether rational modifications made to residues that bind to the HIV-1 CD4 binding site may result in improved neutralizing activity. Modifications made to L1A2 were informed by studies evaluating N6, an antibody with comparable neutralization activity to L1A2 (Huang et al, Immunity 45:1108-1121, 2016). Cd4bs_H53MS_H54GY contains two mutations in H-CDR2 at positions 53 and 54. The first modification is intended to minimize steric clashes with HIV-1 gp120 and improve binding to the CD4 binding site. Studies evaluating the evolution of N6 identified early precursors of the antibody revealing that the lysine at position 53 was modified to a glutamine during the natural development of N6 to minimize steric clashes between the N6 paratope and HIV-1 gp120. This modification, among others, likely contributed to breadth in antibody N6. Similarly, we chose to minimize steric interference between the L1A2 heavy chain and gp120 protein by mutating the methionine at position 53 to a serine (M53S). The second mutation was also informed by structural studies of N6. This work identified an important, space-filling tyrosine at position 54 in the heavy chain. VRC27, also a potent CD4-binding site antibody, similarly includes a large, hydrophobic residue at that position. Consistent with these observations, we similarly modified the glycine at position 54 to a tyrosine (G54Y) (Huang, et al., supra). When evaluated against the expanded virus panel, Cd4bs_H53MS_H54GY demonstrated 100% breadth, as compared to 70% breadth of the parent L1A2 antibody with a geometric mean IC₅₀ for Cd4bs_H53MS_H54GY (0.265 μg/mL) comparable to parent antibody L1A2 (0.253 μg/mL, Table 17). A, G, V, P, L, I, or T are alternative amino acid residues that can replace M at position 53 of the L1A2 heavy chain and F, W, N, H, L, and I are alternative amino acid residues that can replace G at position 54.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

TABLE 16

| L1A2 | | | | | | | | Geometric | |
|---|---|---|---|---|---|---|---|---|---|
| Antibody | | | | | | | | mean* | |
| production | | | | | | | | (IC₅₀ | Breadth |
| specifications | DU172.17 | CAP45.2.00.G3 | CNE2 | REJO4541.67 | X1632_S2_B10 | AC10.0.29 | DU422.1 | μg/mL) | (%) |
| CRO #1, 293 | 0.268 | 0.132 | 0.089 | 0.054 | >50 | >50 | >50 | 0.114 | 57 |
| cells | | | | | | | | | |
| CRO #2, 293 | 0.279 | 0.209 | 0.124 | 0.072 | >50 | >50 | >50 | 0.151 | 57 |
| cells | | | | | | | | | |
| CRO #2, | 0.507 | 0.162 | 0.092 | 0.045 | >50 | >50 | >50 | 0.136 | 57 |
| CHO cells | | | | | | | | | |

*Geometric mean IC50 is calculated using only the values from the neutralized viruses (IC₅₀ ≤ 50 μg/mL)
#Z,47; Breadth is calculated as the fraction of viruses which are neutralized at IC₅₀ values ≤50 μg/mL.

TABLE 17

| | IC₅₀ (μg/mL) | | | | |
|---|---|---|---|---|---|
| Designation | DU172.17 | THRO4156.18 | CAP45.2.00.G3 | CNE20 | REJO4541.67 |
| L1A2 | 0.279 | 1.041 | 0.209 | 0.124 | 0.072 |
| Germ6 | 0.102 | NA | 0.135 | 0.091 | 0.062 |
| Germ12 | 0.267 | NA | 0.063 | 0.244 | 0.092 |
| Germ18 | 0.223 | NA | 0.036 | 0.081 | 0.039 |
| Germ17 | 0.117 | NA | 0.037 | 0.081 | 0.033 |
| Germ23 | 0.182 | NA | 0.035 | 0.066 | 0.037 |
| NglycoSA | 0.110 | 0.929 | 0.037 | 0.050 | 0.040 |
| NglycoND | 18.266 | NA | 0.077 | >50 | 0.062 |
| CysCA | 0.169 | NA | 0.035 | 0.034 | ≤0.020 |
| CysCV | 0.102 | 1.402 | 0.035 | 0.037 | ≤0.020 |
| Hydro2 | 0.131 | NA | 0.039 | 0.049 | ≤0.020 |
| L1A2_g12_SA | 0.088 | NA | 0.042 | 0.062 | 0.028 |
| CA (Combo) | | | | | |
| L1A2_g12_SA_CV | 0.714 | 11.734 | 0.047 | 0.059 | ≤0.020 |
| L1A2_g23_SA_CV | 0.831 | NA | 0.050 | 0.064 | ≤0.020 |
| Cd4bs_H53MS_H54GY | 0.273 | NA | 0.030 | 0.047 | 0.044 |
| 3BNC117 | 1.255 | NA | 2.704 | >50 | 0.070 |

| | IC₅₀ (μg/mL) | | |
|---|---|---|---|
| Designation | X1632_S2_B10 | WITO4160.33 | AC10.0.29 |
| L1A2 | >50 | 0.200 | >50 |
| Germ6 | >50 | NA | >50 |
| Germ12 | >50 | NA | >50 |
| Germ18 | >50 | NA | >50 |
| Germ17 | >50 | NA | >50 |
| Germ23 | >50 | NA | >50 |
| NglycoSA | >50 | 0.179 | >50 |
| NglycoND | >50 | NA | >50 |
| CysCA | >50 | NA | >50 |
| CysCV | >50 | 0.186 | >50 |
| Hydro2 | >50 | NA | >50 |
| L1A2_g12_SA | >50 | NA | >50 |
| CA (Combo) | | | |
| L1A2_g12_SA_CV | >50 | 0.488 | >50 |
| L1A2_g23_SA_CV | >50 | NA | >50 |
| Cd4bs_H53MS_H54GY | 9.87 | 0.150 | 3.00 |
| 3BNC117 | 31.158 | NA | 11.002 |

| | | | 7 virus panel | | Expanded virus panel | |
|---|---|---|---|---|---|---|
| | | | Geometric | | Geometric | |
| | IC₅₀ μg/mL | | mean* | Breadth #Z,47; | mean* | Breadth #Z,47; |
| Designation | ZM233M.PB6 | DU422.1 | (IC₅₀ μg/mL) | (%) | (IC₅₀ μg/mL) | (%) |
| L1A2 | 0.613 | >50 | 0.151 | 57% | 0.253 | 70% |
| Germ6 | NA | >50 | 0.094 | 57% | NA | NA |
| Germ12 | NA | >50 | 0.140 | 57% | NA | NA |
| Germ18 | NA | >50 | 0.071 | 57% | NA | NA |
| Germ17 | NA | >50 | 0.058 | 57% | NA | NA |
| Germ23 | NA | >50 | 0.063 | 57% | NA | NA |
| NglycoSA | 0.151 | >50 | 0.054 | 57% | 0.111 | 70% |
| NglycoND | NA | >50 | 0.444 | 43% | NA | NA |
| CysCA | NA | >50 | 0.045 | 57% | NA | NA |
| CysCV | 0.079 | >50 | 0.040 | 57% | 0.092 | 70% |
| Hydro2 | NA | >50 | 0.047 | 57% | NA | NA |
| L1A2_g12_SA_CA | NA | >50 | 0.051 | 57% | NA | NA |
| (Combo) | | | | | | |
| L1A2_g12_SA_CV | 0.731 | >50 | 0.079 | 57% | 0.288 | 70% |
| L1A2_g23_SA_CV | NA | >50 | 0.085 | 57% | NA | NA |
| Cd4bs_H53MS_H54GY | 0.096 | 0.890 | 0.064 | 100% | 0.265 | 100% |
| 3BNC117 | NA | >50 | 2.411 | 71% | NA | NA |

*Geometric mean IC₅₀ is calculated using only the values from the neutralized viruses (IC₅₀ ≤ 50 μg/mL).
#Z,47; Breadth calculated as the fraction of viruses which are neutralized at IC₅₀ values ≤50 μg/mL.

What is claimed is:

1. An anti-HIV antibody comprising a heavy chain variable (V_{H}) region and a light chain variable (V_{L}) region, wherein:
(a) the V_{H} region comprises at least one substitution in a CDR1 sequence, a CDR2 sequence, or a CDR3 sequence, wherein the CDR1 sequence comprises ²⁵GYRFPDYIIH³⁴ (SEQ ID NO: 59), the CDR2 sequence comprises ⁴⁹WMNPMGGQVNIPWKFQG⁶⁵ (SEQ ID NO: 60), and the CDR3 sequence comprises ⁹⁶VRDRSNGSGKRFESSNWFLDL¹¹⁶ (SEQ ID NO: 61) as numbered with reference to SEQ ID NO:1; and wherein the at least one substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; N, R, Q, S, or A at position 62, D, D, A, or Q at position 101; W, A, or N at position 103; Q, S, or A at positions 105; Q, S, or A at position 106; Y at position 107; and Y or F at position 112; and
(b) the V_{L} region comprises: (i) a CDR1 sequence comprising ²³TGTHNLVS³⁰ (SEQ ID NO: 62), a CDR2 sequence comprising ⁴⁶DFNKRPS⁵² (SEQ ID NO: 63), and a CDR3 sequence comprising ⁸⁵WAYEA⁸⁹ (SEQ ID NO: 64) as numbered with reference to SEQ ID NO:2; or (ii) at least one substitution in the CDR1 sequence, CDR2 sequence, or CDR3 sequence, wherein the at least one substitution is selected from the group consisting of Y at position 28; Q, S, or A at position 49; Q, S, or A at position 50; F or Y at position 85; and N at position 89.

2. An anti-HIV antibody comprising a heavy chain variable (V_{H}) region and a light chain variable (V_{L}) region, wherein:
(a) the V_{H} region comprises: (i) a CDR1 sequence comprising ²⁵GYRFPDYIIH³⁴ (SEQ ID NO: 59), a CDR2 sequence comprising ⁴⁹WMNPMGGQVNIPWKFQG⁶⁵ (SEQ ID NO: 60), and a CDR3 sequence comprising ⁹⁶VRDRSNGSGKRFESSNWFLDL¹¹⁶ (SEQ ID NO: 61) as numbered with reference to SEQ ID NO:1; or (ii) at least one substitution in the CDR1 sequence, the CDR2 sequence, or the CDR3 sequence, wherein the at least one substitution is selected from the group consisting of Y or F at position 49; I, Q, L, S, or A at position 50; S, V, Q, L, A G, P, I, or T at position 53; Y, F, W, N, H, L, or I at position 54; Q, Y, or F at position 61; N, R, Q, S, or A at position 62, D, D, A, or Q at position 101; W, A, or N at position 103; Q, S, or A at positions 105; Q, S, or A at position 106; Y at position 107; and Y or F at position 112; and
(b) the V_{L} region comprises at least one substitution in a CDR1 sequence, a CDR2 sequence, or a CDR3 sequence, wherein the CDR1 sequence comprises ²³TGTHNLVS³⁰ (SEQ ID NO: 62), the CDR2 sequence comprises ⁴⁶DFNKRPS⁵² (SEQ ID NO: 63), and the CDR3 sequence comprises ⁸⁵WAYEA⁸⁹ (SEQ ID NO: 64) as numbered with reference to SEQ ID NO:2; and wherein the at least one substitution in the CDR1 sequence, CDR2 sequence, or CDR3 sequence is selected from the group consisting of Y at position 28; Q, S, or A at position 49; Q, S, or A at position 50; F or Y at position 85; and N at position 89.

3. The anti-HIV antibody of claim 1, wherein the V_{H} region comprises a CDR1 sequence GVTFPDYIIH (SEQ ID NO: 65); a CDR2 sequence WMNPMGGQVNIPQKFQG (SEQ ID NO: 66) or WMNPSYGQVNIPWKFQG (SEQ ID NO: 67); or a CDR3 sequence VRDRSNGAGKRFESSNWFLDL (SEQ ID NO: 68), or VRDRGDGSRRHFDSSNWFLDL (SEQ ID NO: 69); and/or the V_{L} region comprises the CDR3 sequence WAYEN (SEQ ID NO: 70).

4. The anti-HIV antibody of claim 3, wherein the CDR2 sequence comprises
(SEQ ID NO: 67)
WMNPSYGQVNIPWKFQG.

5. The anti-HIV antibody of claim 1, wherein the V_{H} region comprises at least one of the following, as numbered with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126.

6. The anti-HIV antibody of claim 1, wherein the V_{L} region comprises at least one of the following, as numbered with reference to SEQ ID NO:2: G at position 12; Y at position 28; Y, A, V, L, or I at position 32; Q at position 34; H at position 35; K at positon 38; M at position 43; K at position 62; N at position 65; S at position 72; A at position 76; E at position 77; E at position 79; D at position 81; or Y at position 83.

7. The anti-HIV antibody of claim 3, wherein the V_{H} region comprises at least one of the following, as numbered with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and/or the V_{L} region comprises at least one of the following, as numbered with reference to SEQ ID NO:2: G at position 12; Y at position 28; Y, A, V, L, or I at position 32; Q at position 34; H at position 35; K at positon 38; M at position 43; K at position 62; N at position 65; S at position 72; A at position 76; E at position 77; E at position 79; D at position 81; or Y at position 83.

8. The anti-HIV antibody of claim 1, wherein the V_{H} region has at least 70% identity to SEQ ID NO:1; and the VL region has at least 70% identity to SEQ ID NO:2.

9. The anti-HIV antibody of claim 1, wherein the V_{H} region has at least 80% identity to SEQ ID NO:1; and/or the VL region has at least 80% identity to SEQ ID NO:2.

10. The anti-HIV antibody of claim 1, wherein the V_{H} region has at least 90% identity to SEQ ID NO:1; and/or the VL region has at least 90% identity to SEQ ID NO:2.

11. The anti-HIV antibody of claim 1, wherein the V_{H} region has at least 95% identity to SEQ ID NO:1; and/or the VL region has at least 95% identity to SEQ ID NO:2.

12. An anti-HIV antibody comprising a heavy chain variable (V_{H}) region and a light chain variable (V_{L}) region, wherein:
(i) the V_{H} region has at least 70% identity to SEQ ID NO:1 and comprises at least one of the following substitutions as determined with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and
(ii) the V_{L} region comprises the amino acid sequence of SEQ ID NO:2; or
an amino acid sequence having at least 70% identity to SEQ ID NO:2 and at least one of the following substitutions as determined with reference to SEQ ID NO:2: Y at position 28; Q, S, or A at position 49; Q, S, or A at position 50; F or Y at position 85; or N at position 8.

13. An anti-HIV antibody comprising a heavy chain variable (V_{H}) region and a light chain variable (V_{L}) region, wherein:
(a) (i) the V_{H} region comprises the amino acid sequence of SEQ ID NO:1; or
(ii) the V_{H} region has at least 70% identity to SEQ ID NO:1 and comprises at least one of the following substitutions as determined with reference to SEQ ID NO:1: V at position 1, Q at position 2, E at position 9, A at position 15, K at position 18, V at position 19, K at position 22, S at position 24, V at position 36, Q at position 38, L at position 44, T at position 68, T at position 75, S at position 76, Y at position 79, M at position 80, E at position 81, S at position 83, R at position 84, R at position 86, L at position 122, V at position 125, or S at position 126; and
(b) the V_{L} region comprises an amino acid sequence having at least 70% identity to SEQ ID NO:2 and at least one of the following substitutions as determined with reference to SEQ ID NO:2: Y at position 28; Q, S, or A at position 49; Q, S, or A at position 50; F or Y at position 85; or N at position 8.

14. The anti-HIV antibody of claim 12, wherein the V_{H} comprises an amino acid sequence having at least 80% identity to SEQ ID NO:1; and/or the V_{L} region comprises an amino acid sequence having at least 80% identity to SEQ ID NO:2.

15. The anti-HIV antibody of claim 12, wherein the V_{H} comprises an amino acid sequence having at least 90% identity to SEQ ID NO:1; and/or the V_{L} region comprises an amino acid sequence having at least 90% identity to SEQ ID NO:2.

16. An anti-HIV antibody comprising a V_{H} region and V_{L} region, wherein the V_{H} region has at least 90% identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region has at least 90% identity to SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:4, SEQ ID NO:46, SEQ ID NO:48, SEQ ID NO:50, SEQ ID NO:52, SEQ ID NO:54, SEQ ID NO:56, or SEQ ID NO:58.

17. The anti-HIV antibody of claim 16, where in the VH region has at least 95% identity to SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region has at least 95% identity to SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:4, SEQ ID NO:46, SEQ ID NO:48, SEQ ID NO:50, SEQ ID NO:52, SEQ ID NO:54, SEQ ID NO:56, or SEQ ID NO:58.

18. The anti-HIV antibody of claim 16, wherein the VH region comprises an amino acid sequence SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, or SEQ ID NO:57; and/or the VL region comprises an amino acid sequence SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:4, SEQ ID NO:46, SEQ ID NO:48, SEQ ID NO:50, SEQ ID NO:52, SEQ ID NO:54, SEQ ID NO:56, or SEQ ID NO:58.

19. The anti-HIV antibody of claim 16, wherein the antibody comprises:
a V_{H} region comprising amino acid sequence SEQ ID NO:3; and a V_{L} region comprising amino acid sequence SEQ ID NO:4;
a V_{H} region comprising amino acid sequence SEQ ID NO:5; and a V_{L} region comprising amino acid sequence SEQ ID NO:6;
a V_{H} region comprising amino acid sequence SEQ ID NO:7; and a V_{L} region comprising amino acid sequence SEQ ID NO:8;
a V_{H} region comprising amino acid sequence SEQ ID NO:9; and a V_{L} region comprising amino acid sequence SEQ ID NO:10;
a V_{H} region comprising amino acid sequence SEQ ID NO:11; and a V_{L} region comprising amino acid sequence SEQ ID NO:12;
a V_{H} region comprising amino acid sequence SEQ ID NO:13; and a V_{L} region comprising amino acid sequence SEQ ID NO:14;
a V_{H} region comprising amino acid sequence SEQ ID NO:15; and a V_{L} region comprising amino acid sequence SEQ ID NO:16;
a V_{H} region comprising amino acid sequence SEQ ID NO:17; and a V_{L} region comprising amino acid sequence SEQ ID NO:18;
a V_{H} region comprising amino acid sequence SEQ ID NO:19; and a V_{L} region comprising amino acid sequence SEQ ID NO:20;
a V_{H} region comprising amino acid sequence SEQ ID NO:21; and a V_{L} region comprising amino acid sequence SEQ ID NO:22;
a V_{H} region comprising amino acid sequence SEQ ID NO:23; and a V_{L} region comprising amino acid sequence SEQ ID NO:24;
a V_{H} region comprising amino acid sequence SEQ ID NO:25; and a V_{L} region comprising amino acid sequence SEQ ID NO:26;
a V_{H} region comprising amino acid sequence SEQ ID NO:27 and a VL region comprising amino acid sequence SEQ ID NO:28;
a VH region comprising amino acid sequence SEQ ID NO:29 and a VL region comprising amino acid sequence SEQ ID NO:30;
a VH region comprising amino acid sequence SEQ ID NO:31 and a VL region comprising amino acid sequence SEQ ID NO:32;
a VH region comprising amino acid sequence SEQ ID NO:33 and a VL region comprising amino acid sequence SEQ ID NO:34;
a VH region comprising amino acid sequence SEQ ID NO:35 and a VL region comprising amino acid sequence SEQ ID NO:36;
a VH region comprising amino acid sequence SEQ ID NO:27 and a VL region comprising amino acid sequence SEQ ID NO:38;
a VH region comprising amino acid sequence SEQ ID NO:39 and a VL region comprising amino acid sequence SEQ ID NO:40;
a VH region comprising amino acid sequence SEQ ID NO:41 and a VL region comprising amino acid sequence SEQ ID NO:42;
a VH region comprising amino acid sequence SEQ ID NO:43 and a VL region comprising amino acid sequence SEQ ID NO:44;
a VH region comprising amino acid sequence SEQ ID NO:45 and a VL region comprising amino acid sequence SEQ ID NO:46;
a VH region comprising amino acid sequence SEQ ID NO:47 and a VL region comprising amino acid sequence SEQ ID NO:48;
a VH region comprising amino acid sequence SEQ ID NO:49 and a VL region comprising amino acid sequence SEQ ID NO:50;
a VH region comprising amino acid sequence SEQ ID NO:51 and a VL region comprising amino acid sequence SEQ ID NO:52;
a VH region comprising amino acid sequence SEQ ID NO:53 and a VL region comprising amino acid sequence SEQ ID NO:54;
a VH region comprising amino acid sequence SEQ ID NO:55 and a VL region comprising amino acid sequence SEQ ID NO:56; or
a VH region comprising amino acid sequence SEQ ID NO:57 and a VL region comprising amino acid sequence SEQ ID NO:58.

20. The antibody of claim 19, wherein the antibody comprises:
a V_{H} region comprising amino acid sequence SEQ ID NO:3; and a V_{L} region comprising amino acid sequence SEQ ID NO:4;
a V_{H} region comprising amino acid sequence SEQ ID NO:5; and a V_{L} region comprising amino acid sequence SEQ ID NO:6;
a V_{H} region comprising amino acid sequence SEQ ID NO:7; and a V_{L} region comprising amino acid sequence SEQ ID NO:8;
a V_{H} region comprising amino acid sequence SEQ ID NO:9; and a V_{L} region comprising amino acid sequence SEQ ID NO:10;
a V_{H} region comprising amino acid sequence SEQ ID NO:11; and a V_{L} region comprising amino acid sequence SEQ ID NO:12;
a V_{H} region comprising amino acid sequence SEQ ID NO:13; and a V_{L} region comprising amino acid sequence SEQ ID NO:14;
a V_{H} region comprising amino acid sequence SEQ ID NO:15; and a V_{L} region comprising amino acid sequence SEQ ID NO:16;
a V_{H} region comprising amino acid sequence SEQ ID NO:17; and a V_{L} region comprising amino acid sequence SEQ ID NO:18;
a V_{H} region comprising amino acid sequence SEQ ID NO:19; and a V_{L} region comprising amino acid sequence SEQ ID NO:20;
a V_{H} region comprising amino acid sequence SEQ ID NO:21; and a V_{L} region comprising amino acid sequence SEQ ID NO:22;
a V_{H} region comprising amino acid sequence SEQ ID NO:23; and a V_{L} region comprising amino acid sequence SEQ ID NO:24;
a V_{H} region comprising amino acid sequence SEQ ID NO:25; and a V_{L} region comprising amino acid sequence SEQ ID NO:26;
a V_{H} region comprising amino acid sequence SEQ ID NO:27 and a VL region comprising amino acid sequence SEQ ID NO:28;
a VH region comprising amino acid sequence SEQ ID NO:35 and a VL region comprising amino acid sequence SEQ ID NO:36; or
a VH region comprising amino acid sequence SEQ ID NO:57 and a VL region comprising amino acid sequence SEQ ID NO:58.

21. An expression vector comprising a polynucleotide encoding the V_{H} region and/or the V_{L} region of the anti-HIV antibody of claim 1.

22. A host cell that comprises an expression vector of claim 21.

23. A host cell comprising a polynucleotide that encodes the V_{H} region and/or the V_{L} region of the anti-HIV antibody of claim 1.

24. A method of treating or preventing an HIV infection, the method comprising administering the anti-HIV antibody of claim 1 to a patient that is infected with an HIV virus, or is at risk of infection of with an HIV virus.

25. The method of claim 24, further comprising administering a latency reversing agent.
